(19) United States
(12) Patent Application Publication
(10) Pub. No.: US 2024/0000694 A1
(43) Pub. Date: Jan. 4, 2024

(54) Methods and Materials for Matching Chemistry of Individuals and Human Melanin

(71) Applicant: Northwestern University, Evanston, IL (US)

(72) Inventors: Nathan C. GIANNESCHI, Wilmette, IL (US); Zofia Elzbieta SIWICKA, Evanston, IL (US); Wei CAO, Evanston, IL (US)

(21) Appl. No.: 18/215,701

(22) Filed: Jun. 28, 2023

Related U.S. Application Data
(60) Provisional application No. 63/357,713, filed on Jul. 1, 2022.

(51) Int. Cl. A61K 8/81; A61Q 5/10; A61K 8/02; G01N 33/68

(52) U.S. Cl. CPC A61K 8/817; A61Q 5/10; A61K 8/0279; G01N 33/6848; A61K 2800/43; G01N 2333/908

(57) ABSTRACT

Aspects disclosed herein include a method for matching hair composition, the method comprising: characterizing one or more first characteristics of a natural melanin composition of a hair sample from a subject; and preparing a prepared artificial melanin formulation to approximate (or, to match or to resemble) the one or more first characteristics; wherein the prepared artificial melanin formulation comprises one or more artificial melanin materials.

### CROSS-REFERENCE TO RELATED APPLICATIONS

This application claims the benefit of priority to U.S. Provisional Patent Application No. 63/357,713, filed Jul. 1, 2022, which is hereby incorporated by reference in its entirety.

### STATEMENT REGARDING FEDERALLY SPONSORED RESEARCH OR DEVELOPMENT

This invention was made with government support under Award Number FA9550-18-1-0142 awarded by the Air Force Office of Scientific Research (AFOSR). The government has certain rights in the invention.

### BACKGROUND OF INVENTION

From countering the effects of aging or illness to expressing one's uniqueness, people have dyed their hair for a myriad of reasons throughout the ages. There are various conventional methods and agents for dying hair, some of which are permanent dyes and others are semi-permanent, for example. Due to such widespread use, hair dye industries are now among the most profitable in the cosmetics sector. Some studies suggest that over 50% of the population in developed countries has dyed their hair at least once in their life.

Melanin is a pigment that is responsible for the color of human hair. Eumelanin, a brown and black pigment, is the most common form of melanin in humans. Pheomelanin, a red pigment, is also found in humans but is less common. The loss of melanin pigment in hair leads to hair whitening. Many commercial products have been developed to dye hair in a variety of colors including those not naturally occurring. However, conventional commercial products suffer from requiring long application times, harsh, potentially carcinogenic, reagents, poor persistence, allergic reactions to reagents, and/or poor coloration.

As an alternative approach to the use of conventional commercial products, a synthetic version of melanin, which matches the exact chemical signature and color to the natural sample could be synthesized. However, such an approach would require determining the chemical signature of the melanin in natural hair and developing a synthetic strategy for preparing a synthetic melanin which matches the chemical signature of the melanin. Such a technique would engineer an exact melanin mimic biocompatible hair dye that would not have any of the adverse effects that are associated with commercial hair dyes.

It is thus apparent that there is need in the art for new methods of characterizing natural melanin compositions from hair samples and preparing an artificial melanin formulation to approximate (or, to match or to resemble) one or more characteristics of the natural melanin compositions. This disclosure provides such methods. Other aspects and benefits of the inventive methods will be readily apparent from the disclosure provided herein.

### SUMMARY OF THE INVENTION

Included herein are methods for matching hair compositions. The methods include, inter alia, characterizing a natural melanin composition of a hair sample from a subject; and preparing an artificial melanin formulation to approximate (or, to match or to resemble) one or more characteristics of the natural melanin composition; wherein the prepared artificial melanin formulation comprises one or more artificial melanin materials. In some embodiments, the methods may include preparing synthetic melanin derivatives by comparison with enzymatically extracted natural melanin derivative samples from various sources utilizing non-destructive characterization methods.

Aspects disclosed herein include a method for matching hair composition, the method comprising: characterizing one or more first characteristics of a natural melanin composition of a hair sample from a subject; and preparing a prepared artificial melanin formulation to approximate (or, to match or to resemble) the one or more first characteristics; wherein the prepared artificial melanin formulation comprises one or more artificial melanin materials.

Aspects disclosed herein include a method for matching hair composition, the method comprising: characterizing one or more first characteristics of a natural melanin composition of a hair sample from a subject; determining (or, designing) a theoretical artificial melanin formulation to approximate (or, to match or to resemble) the one or more characteristics of the natural melanin composition; preparing a prepared artificial melanin formulation according to (or, to match or to resemble) the theoretical artificial melanin composition; wherein the prepared artificial melanin formulation comprises one or more artificial melanin materials.

Aspects disclosed herein include a method for matching melanin composition, the method comprising: characterizing one or more first characteristics of a natural melanin composition of a biological sample from a subject; and preparing a prepared artificial melanin formulation to approximate (or, to match or to resemble) the one or more first characteristics; wherein the prepared artificial melanin formulation comprises one or more artificial melanin materials.

Aspects disclosed herein include a method for matching melanin composition, the method comprising: characterizing one or more first characteristics of a natural melanin composition of a biological sample from a subject; determining (or, designing) a theoretical artificial melanin formulation to approximate (or, to match or to resemble) the one or more characteristics of the natural melanin composition; preparing a prepared artificial melanin formulation according to (or, to match or to resemble) the theoretical artificial melanin composition; wherein the prepared artificial melanin formulation comprises one or more artificial melanin materials.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B: Chemical synthesis of pheomelanin using different methods. FIG. 1A: Method 1) KMnO₄ was used to oxidize a solution phase mixture of cysteine and _{L}-DOPA in water at pH 7; Method 2) Oxygen was used to oxidize cysteine and _{L}-DOPA in phosphate buffered saline (PBS) in the presence of tyrosinase; Method 3) 5-cysteinyl-DOPA (5-CD) was used as the starting material under chemoenzymatic oxidation with horseradish peroxidase (HRP) and H₂O₂. FIG. 1B: ¹³C ssNMR spectra of the pheomelanin samples synthesized from the three methods. Data for method 1 and 2 were reproduced from our previous work²³.

FIGS. 2A-2E: Chemical synthesis of pheomelanin using 5-cysteinyl-DOPA (5-CD) as the starting material. FIG. 2A: The color change at various stages of the reaction. FIG. 2B: The kinetic trace shows the continuous progress of the reaction at 500 nm. a.u., arbitrary units. FIG. 2C: Representative SEM image of the synthetic pheomelanin. FIG. 2D: FTIR spectra and (FIG. 2E)¹³C ssNMR spectra of 5-CD pheomelanin overlaid with 5-CD monomer.

FIGS. 3A-3F: Natural pheomelanins extracted from bird feathers and human red hair from two individuals. FIGS. 3A-3C: Optical images and SEM images of natural pheomelanin samples extracted from rooster feathers, human red hair I, and human red hair II, respectively. FIGS. 3D-3F: DLS plots of the corresponding natural pheomelanins. Inset are the optical images of the pheomelanin dispersion.

FIGS. 4A-4C: Comparison of synthetic pheomelanin with natural pheomelanin. Four samples were used in this experiment: synthetic 5-CD pheomelanin, pheomelanin extracted from rooster feathers and human red hair from two individuals. FIG. 4A: ssNMR spectra overlay of the synthetic and natural pheomelanin. Inset is the zoomed aromatic region. FIG. 4B: FTIR spectra and (FIG. 4C) UV-vis absorption spectra of the synthetic and natural pheomelanin.

FIGS. 5A-5I: Surface properties and color comparison of synthetic pheomelanin with PDA mimics of eumelanin. The static water contact angle images for (FIG. 5A) pheomelanin film, (FIG. 5B) PDA film and (FIG. 5C) blank glass. Surface morphology by SEM for (FIG. 5D) pheomelanin film, (FIG. 5E) PDA film and (FIG. 5F) blank glass. FIG. 5G: Optical photos show the color changes before and after pheomelanin coating on planar substrates, including glass, polystyrene and gold substrates. FIG. 5H: 3D-printed objects before and after the coating with synthetic eumelanin and pheomelanin. FIG. 5I: The color of synthetic eumelanin and pheomelanin dispersion at 1 mg/mL concentration.

FIGS. 6A-6D: Paramagnetic properties comparison of synthetic pheomelanin with PDA mimics of eumelanin and natural pheomelanin. FIG. 6A: EPR spectra under attenuation powers of 30 dB. FIG. 6B: Power saturation curves for the three samples. FIG. 6C: The T₁ relaxation time and fitted plot for the three samples. Open circles are the pulse EPR experimental values, and the solid lines are the fitted curves. FIG. 6D: Table of the summarized values of spin concentration, P_{1/2} and T₁ relaxation time.

FIGS. 7A-7E: Cation-π interactions in the synthetic pheomelanin. FIG. 7A: A schematic description for cation-π interaction and alkali-triggered disassembly of the pheomelanin film. FIG. 7B: The optical images showcase the color change of the pristine pheomelanin film, and films after treatment with pH 10 solution, NaCl (0.5 M), and KCl (0.5 M) solution, respectively. FIG. 7C: UV-vis spectra of each pheomelanin film sample. a.u., arbitrary units. FIG. 7D: Absorbance comparison at 400 nm and statistical test. Error bars represent the standard deviation of >4 measurements in a single experiment. NS means no statistical difference P>0.1, ***: P<0.001. FIG. 7E: Representative SEM micrographs of each pheomelanin film. Scale bar is 1 μm and applies to all images in the set.

FIGS. 8A-8B: Synthetic attempts to generate pheomelanin using L-DOPA, and cystine as the starting materials. FIG. 8A: To circumvent the cysteine dimerization in air, KMnO₄ was used to oxidize a solution phase mixture of cysteine and L-DOPA in water at pH 7, following the in situ reduction of cystine. FIG. 8B: ¹³C ssNMR of the black powders from this method showed similar sharp peaks to the KMnO₄ and tyrosinase method. The aromatic peaks were barely observed in this case.

FIGS. 9A-9D: Synthetic route and solution NMR characterization of 5-CD monomer. FIG. 9A: Synthesis of 5-CD. FIG. 9B: ¹H NMR. FIG. 9C: ¹³C NMR. FIG. 9D: ¹⁹F NMR spectra of 5-CD in D₂O.

FIGS. 10A-10H: XPS, ESI-MS, and ssNMR characterization of 5-CD monomer. FIGS. 10A-10F: XPS spectra of 5-CD. FIG. 10A: Wide-scan XPS survey spectrum. FIG. 10B: Deconvoluted C1s spectrum. FIG. 10C: F1s spectrum. FIG. 10D: S2p spectrum. FIG. 10E: O1s spectrum, FIG. 10F: N1s spectrum. FIG. 10G: ESI-MS spectra of 5-CD. Solvent: water. [M+H]⁺ calculated 317.08, found 317.06, [2M+H]⁺ calculated 633.15, found 633.06, [3M+H]⁺ calculated 949.23, found 949.16. FIG. 10H: ssNMR spectra of 5-CD before (lower) and after (upper) HPLC purification. The extra peak at 163.0 ppm after HPLC purification is assigned to the trifluoroacetate counterion, corresponding to the ¹⁹F NMR in FIGS. 9A-9D and the XPS F 1s signal in FIG. 10C.

FIGS. 11A-11D: Polymerization reaction of 5-CD to prepare synthetic pheomelanin. FIG. 11A: HPLC analysis of the reaction at various stages. FIG. 11B: The reaction intermediates identified by ESI-MS spectra of the fractions from the HPLC. FIG. 11C: UV-vis spectroscopy monitoring of the reaction. FIG. 11D: Zoom of UV-vis spectroscopy from 250 nm to 700 nm.

FIGS. 12A-12I: STEM images with sulfur element EDS mapping of the feather pheomelanin (FIGS. 12A, 12D, 12H), synthetic pheomelanin (FIGS. 12B, 12E, 12I) and control L-DOPA NP (FIGS. 12C, 12F, 12G). Sulfur-free L-DOPA NP control was made by polymerization of L-DOPA using KMnO₄ as the oxidative agent. FIGS. 12A-12C, TEM images, FIGS. 12D-12F, sulfur mapping, FIGS. 12G-12I, the overlay of mapping and TEM. Sulfur signal is false colored with yellow.

FIGS. 13A-13I: Characterization of the 5-CD synthetic pheomelanin. FIG. 13A: Optical photograph of the synthetic pheomelanin powder. FIG. 13B: Normalized UV-vis plot of the synthetic pheomelanin suspension. FIG. 13C: DLS plot of the synthetic pheomelanin sample. D-1. XPS spectra of synthetic pheomelanin. FIG. 13D: Wide-scan XPS survey spectrum. FIG. 13E: Deconvoluted C1s spectrum. FIG. 13F: F1s spectrum shows that the TFA counterion diminished after the polymerization, corresponding to the intramolecular cyclization of amine to form the benzothiazine structure. FIG. 13G: S2p spectrum. FIG. 13H: O1s spectrum. FIG. 13I: N1s spectrum.

FIGS. 14A-14E: DLS and ssNMR comparison of the synthetic and natural pheomelanin samples. FIG. 14A: Hydrodynamic radii and Zeta potentials of the synthetic pheomelanin and natural pheomelanin. FIG. 14B: The ssNMR spectrum of pheomelanin from human hair sample 1. FIG. 14C: Reproduced from previous literature result of enzymatically extracted pheomelanin from human hair.⁷ FIG. 14D: The ssNMR spectrum of pheomelanin from human red hair I after retreatment with proteinase K. FIG. 14E: The ssNMR spectrum of 5-CD pheomelanin irradiated with UVA. The pristine 5-CD pheomelanin was plotted here for reference.

FIGS. 15A-15D: Absorption and reflectance spectra of the melanin films. FIG. 15A: UV-vis absorption spectra comparison of 5-CD pheomelanin suspension and film. FIG. 15B: UV-vis absorption spectra of 5-CD pheomelanin film before and after treatment with 1% acetic acid. FIG. 15C: Reflectance spectrum for the films made using 5-CD pheomelanin, and pheomelanin from bird feathers. FIG. 15D: Reflectance spectrum for the PDA type eumelanin film.

FIGS. 16A-16F: EPR quantification of the radical content in pheomelanin. FIG. 16A: EPR spectra of 4-amino-TEMPO standard solutions of 500, 100 and 10 μM. FIG. 16B: Integration plots of EPR spectra in A. Plots were baseline corrected in Origin software. FIG. 16C: Integrated EPR spectra of B. FIG. 16D: The EPR calibration curve of double integration area vs spin concentration. FIGS. 16E-16F: EPR spectrum of feather pheomelanin (FIG. 16E) and 5-CD pheomelanin (FIG. 16F) in aqueous dispersion.

FIGS. 17A-17H: 5-CD Pheomelanin films after treatment with different solutions at pH 10. Optical images and absorbance mapping at 400 nm on glass slide substrates are shown. Here substrates with small areas are used to facilitate screening. FIG. 17A: Optical images of the pheomelanin film treated with KCl solution (pH 10) at various concentrations, including 0 M, 0.1 M, 0.5 M and 1.0 M. FIG. 17B: Optical images of the pheomelanin film treated with NaCl solution (pH 10) at various concentrations, including 0 M, 0.1 M, 0.5 M and 1.0 M. FIGS. 17C-17H are the optical images and the corresponding absorbance mapping at 400 nm. FIG. 17C: The pristine pheomelanin film. FIG. 17D: Film treated with 0.5 M KCl solution (pH 10). FIG. 17E: Film treated with pH 10 KOH solution. FIG. 17F: Film treated with 0.5 M KBr solution (pH 10). FIG. 17G: Film treated with 0.5 M NaCl solution (pH 10). FIG. 17H Film treated with 0.5 M K₂SO₄ solution (pH 10).

### STATEMENTS REGARDING CHEMICAL COMPOUNDS AND NOMENCLATURE

In general, the terms and phrases used herein have their art-recognized meaning, which can be found by reference to standard texts, journal references and contexts known to those skilled in the art. The following definitions are provided to clarify their specific use in the context of the invention.

The term “spectral matching” refers to a qualitative and/or quantitative way of assessing the similarity of two data sets (e.g., spectra). The two data sets resemble each other by having at least 70% (e.g., at least 75%, at least 80%, at least 85%, at least 90%, at least 95%, or 100%) of the same characteristic peaks. Alternatively, or in addition too, two data points (e.g., peaks, values, signals, etc.) must be within 10% error (e.g., within 8% error, within 5% error, within 3% error, or within 1% error) to be considered equivalent data points (e.g., peaks, values, signals, etc.).

The term “formulation” refers to a melanin (e.g., a melanin derivative) composition, which comprises melanin or a derivative thereof, melanin precursors (e.g., materials utilized in the synthesis of melanin), or additional additives such as solvents (e.g., carriers), lubricants, dispersants, oils, fragrances, natural ingredients, pharmaceutically acceptable excipients, etc.

The term “subject” or “patient” refers to a living organism. Non-limiting examples include humans, other mammals, bovines, rats, mice, dogs, monkeys, goat, sheep, cows, deer, and other non-mammalian animals. The subject may optionally but not necessarily be suffering from or having a wound, disease, or condition that can be treated or remediated, at least in part, by administration of a formulation or melanin material as provided herein. In some embodiments, such as some of Aspects 1-39, a subject is human. In some embodiments, such as some of Aspects 1-39, a subject is a mammal. In some embodiments, such as some of Aspects 1-39, a subject is a mouse. In some embodiments, such as some of Aspects 1-39, a subject is an experimental animal. In some embodiments, such as some of Aspects 1-39, a subject is a rat. In some embodiments, such as some of Aspects 1-39, a subject is a test animal.

The term “melanin” generally refers to one or more compounds or materials that function as a pigment, such as when internalized or taken up by a biological cell, for example. It is also noted that melanin is not necessarily taken up by cells. Melanin can be incorporated in or on cell walls in fungi, for example, such as to provide rigidity, defense mechanisms, and more. In another illustrative example, melanin is used by birds, such as where melanin is organized in a matrix of keratin or similar type of biological material, where it can be organized into monolayers or multilayers to provide structural color, warmth, and more. A melanin compound or material may be, but is not limited to, a melanin monomer, a melanin oligomer, a melanin polymer, a melanin nanoparticle, a melanin layer (e.g., a melanin thin film or coating), or other melanin material, for example. For example, melanin nanoparticles internalized by a biological cell function as a pigment in the cell.

The terms “artificial melanin” and “synthetic melanin” are used interchangeably herein and refer to one or more melanin compounds, molecules, or materials, such as melanin monomers, melanin oligomers, or melanin nanoparticles, that are synthesized and are at least partially, or preferably entirely, not derived from or not extracted from a natural source, such as a biological source, a living organism, or a once living organism. The terms “synthetic” and “artificial” are used interchangeably herein when referring to a melanin or a material comprising a melanin. The terms “synthetic melanin nanoparticles” and “artificial melanin nanoparticles” are used interchangeably herein, and are intended to have the same meaning throughout the present disclosure, and refer to nanoparticles formed of artificial melanin, such as artificial melanin monomers and/or artificial melanin oligomers. The terms “synthetic melanin thin film” and “artificial melanin thin film” are used interchangeably herein, and are intended to have the same meaning throughout the present disclosure, and refer to a thin film formed of artificial melanin, such as artificial melanin monomers and/or artificial melanin oligomers. The terms “synthetic melanin layer” and “artificial melanin layer” are used interchangeably herein, and are intended to have the same meaning throughout the present disclosure, and refer to a layer formed of artificial melanin, such as artificial melanin monomers and/or artificial melanin oligomers. An artificial melanin nanoparticle, artificial melanin thin film, artificial melanin layer, and any compound, material, or formulation comprising any of these, comprises artificial melanin monomers, artificial melanin oligomers, and/or artificial melanin polymers. Optionally, an artificial melanin nanoparticle, artificial melanin thin film, artificial melanin layer, and any compound, material, or formulation comprising any of these, consists of or consists essentially of artificial melanin, such as artificial melanin monomers, artificial melanin oligomers, and/or artificial melanin polymers. Optionally, an artificial melanin nanoparticle, artificial melanin thin film, artificial melanin layer, and any compound, material, or formulation comprising any of these, is free (or substantially free) of artificial melanin monomers and comprises artificial melanin oligomers and/or artificial melanin polymers. Preferably, each artificial melanin monomer, artificial melanin oligomer, and artificial melanin polymer of an artificial melanin nanoparticle, artificial melanin thin film, artificial melanin layer, and any compound, material, or formulation comprising any of these, is not bound to, conjugated to, attached to, coated by, encompassed by or chemically otherwise associated with a natural or biological proteinaceous lipid. A natural or biological proteinaceous lipid refers to a naturally or biologically derived lipid or a lipid extracted from a natural or biological source, such as a once living organism, said lipid comprising one or more proteins such as the lipid (plasma) membrane of a melanocyte or melanosome). Optionally, each artificial melanin monomer, artificial melanin oligomer, and artificial melanin polymer of an artificial melanin nanoparticle, artificial melanin thin film, artificial melanin layer, and any compound, material, or formulation comprising any of these, is not bound to, conjugated to, attached to, coated by, encompassed by or otherwise chemically associated with a natural or biological lipid (e.g. a lipid bilayer, lipid membrane or phospholipid compound). A natural or biological lipid refers to a naturally or biologically derived lipid or a lipid extracted from a natural or biological source, such as a once living organism. Optionally, any artificial melanin monomer, artificial melanin oligomer, and artificial melanin polymer of an artificial melanin nanoparticle, artificial melanin thin film, artificial melanin layer, and any compound, material, or formulation comprising any of these, is bound to, conjugated to, attached to, coated by, encompassed by, and/or otherwise associated with a synthetic or artificial lipid or with a synthetic or artificial phospholipid. A synthetic or artificial lipid refers to a synthesized lipid that is not derived from or is not extracted from a natural or biological source, such as a once living organism.

The term “artificial melanin precursor” refers to a compound or material that can form an artificial melanin material after a chemical reaction, such as after a chemical reaction with an oxidation agent. An artificial melanin precursor can be, but is not necessarily, itself a melanin. For example, an artificial melanin precursor can be, but is not necessarily, a melanin monomer. For example, contacting artificial melanin precursors such as melanin monomers with an oxidizing agent can result in oxidative oligomerization (or, polymerization) among the artificial melanin precursors thereby forming artificial melanin material(s).

The term “selenomelanin” refers to melanin comprising selenium. For example, a selenomelanin material comprises selenium. Preferably, a chemical formula of a selenomelanin material comprises selenium (e.g., at least one selenium atom).

In certain embodiments, the term “pheomelanin” refers to a melanin whose chemical formula comprises at least one substituted or unsubstituted benzothiazine, at least one substituted or unsubstituted benzothiazole, at least one substituted or unsubstituted benzoselenazole, at least one substituted or unsubstituted benzoselenazine, at least one derivative of any of these, or any combination of these. In certain embodiments, the term pheomelanin refers to a melanin made from L-DOPA and cysteine, whose chemical formula comprises at least one substituted or unsubstituted benzothiazine, at least one substituted or unsubstituted benzothiazole, at least one substituted or unsubstituted benzoselenazole, at least one substituted or unsubstituted benzoselenazine, at least one derivative of any of these, or any combination of these. In certain embodiments, a selenium pheomelanin refers to a melanin whose chemical formula comprises at least one substituted or unsubstituted benzoselenazole, at least one substituted or unsubstituted benzoselenazine, at least one derivative of any of these, or any combination of these.

In certain embodiments, the term eumelanin refers to a melanin whose chemical formula comprises at least one dihydoxyindole (DHI) (e.g., 5,6-dihydroxyindole), at least one dihydroxyindole-2-carboxylic acid (DHICA) (e.g., 5,6-dihydroxyindole-2-carboxylic acid), or a combination of these.

The term “nanoparticle” as used herein, refers to a physical particle having at least one size characteristic or physical dimension less than less than 1 μm. Preferably, term “nanoparticle” as used herein, refers to a physical particle whose longest size characteristic or physical dimension is less than 1 μm.

The term “size characteristic” refers to a property, or set of properties, of a particle that directly or indirectly relates to a size attribute. According to some embodiments, a size characteristic corresponds to an empirically-derived size characteristic of a particle(s) being detected, such as a size characteristic based on, determined by, or corresponding to data from any technique or instrument that may be used to determine a particle size, such as electron microscope (e.g., SEM and TEM) or a light scattering technique (e.g., DLS). For example, a size characteristic can correspond to a spherical particle exhibiting similar or substantially same properties, such as aerodynamic, hydrodynamic, optical, and/or electrical properties, as the particle(s) being detected). According to some embodiments, a size characteristic corresponds to a physical dimension, such as a cross-sectional size (e.g., length, width, thickness, or diameter).

The term “particles” refers to small solid objects that may be dispersed and/or suspended in a fluid (e.g., liquid). For example, a slurry, a dispersion, and a suspension each include particles in a fluid. The terms “particle” and “particulate” may be used interchangeably. An exemplary particle is an artificial melanin nanoparticle. A plurality of particles may be associated together to form an agglomerate of particles. Generally, the term “particle”, such as “nanoparticle” or “melanin nanoparticle”, refers to an individual particle rather than to an agglomerate of such individual particles.

The term “dispersed” refers to species, such as particles, in a fluid forming a dispersion. As used herein, the term “dispersion” broadly refers to a mixture of one or more chemical species, such as particles, in a fluid, such as the art-recognized meaning of solution, dispersion, and/or suspension. The chemical species, such as particles, dispersed in a dispersion can be referred as a dispersed species. Preferably, a dispersion is a mixture of particles, such as artificial melanin particles, in a liquid, such as a solvent. Preferably, but not necessarily, a dispersion is a homogeneous mixture. In the context of a dispersion, the term “homogeneous” refers to a liquid mixture that appears uniform to the naked eye. In contrast, a heterogeneous liquid mixture includes particles that are precipitated from or suspended in the liquid mixture and are large enough to be distinctly identifiable by the naked eye in the liquid mixture. A heterogeneous liquid mixture includes, for example, sedimented and/or sedimenting particles. Preferably, but not necessarily, the term “dispersion” is broadly intended to include solutions and dispersions, such as colloids, which are not heterogenous liquid mixtures. Preferably, but not necessarily, a dispersion is a microscopically homogenous, or uniform, mixture of particles in a liquid, such as a solvent. Preferably, but not necessarily, a dispersion is thermodynamically favored remain stably dispersed or is thermodynamically favored to segregate by sedimentation but wherein sedimentation is kinetically slowed or prevented. Particles, of a dispersion, that are characterized as stably dispersed remain dispersed in the dispersion and do not sediment or precipitate out of the liquid, of the dispersion, for at least 5 hours, preferably at least 12 hours, preferably at least 24 hours, and more preferably at least 1 week, under normal temperature and pressure (NTP) and exposure to air. In embodiments, particles that are not or cannot be dispersed in a fluid refer to particles that form precipitates or sediments upon being mixed in the fluid.

When referring to a material, such as a polymer, being aqueous, the term “aqueous” refers to said material being dispersed, dissolved, or otherwise solvated by water. An “aqueous solution” refers to a solution that comprises water as solvent and one or more solute species dispersed, dissolved, or otherwise solvated by the water. An aqueous process, such as a polymerization, is a process taking place in an aqueous solution. Optionally, but not necessarily, an aqueous solution or an aqueous solvent includes 20 vol. % or less, optionally 15 vol. % or less, optionally 10 vol. % or less, preferably 5 vol. % or less, of a non-water or organic species. Optionally, but not necessarily, an aqueous solution or an aqueous solvent includes 20 vol. % or less, optionally 15 vol. % or less, optionally 10 vol. % or less, preferably 5 vol. % or less, of a non-water liquid.

The term “peak size” size refers to the statistical mode, or peak frequency, of a particle size distribution, or the particle size most commonly found in the particle size distribution. A particle size distribution can be measured using dynamic light scattering, for example.

The term “sphere” as used herein, in the usual and customary sense, refers to a round or substantially round geometrical object in three-dimensional space that is substantially the surface of a completely round ball, analogous to a circular object in two dimensions. A sphere may be defined mathematically as the set of points that are all at the same or substantially all at the same distance r from a given point, but in three-dimensional space, where r is the radius of the mathematical ball and the given point is the center or substantially the center of the mathematical ball. In embodiments, the longest straight line through the ball, connecting two points of the sphere, passes through the center and its length is thus twice the radius; it is a diameter of the ball. A nanosphere is a nanoparticle having a radius of less than 1 μm.

The terms “reactive oxygen species” and “ROS” as used interchangeably herein refer, in the usual and customary sense, to transient species, typically formed during exposure to radiation (e.g., UV irradiation) capable of inducing oxidative decomposition.

The terms “cell” and “biological cell” are used interchangeably are refer to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. A “viable cell” is a living biological cell.

The term “substantially” refers to a property, condition, or value that is within 20%, 10%, within 5%, within 1%, optionally within 0.1%, or is equivalent to a reference property, condition, or value. The term “substantially equal”, “substantially equivalent”, or “substantially unchanged”, when used in conjunction with a reference value describing a property or condition, refers to a value that is within 20%, within 10%, optionally within 5%, optionally within 1%, optionally within 0.1%, or optionally is equivalent to the provided reference value. For example, a diameter is substantially equal to 100 nm (or, “is substantially 100 nm”) if the value of the diameter is within 20%, optionally within 10%, optionally within 5%, optionally within 1%, within 0.1%, or optionally equal to 100 nm. The term “substantially greater”, when used in conjunction with a reference value describing a property or condition, refers to a value that is at least 1%, optionally at least 5%, optionally at least 10%, or optionally at least 20% greater than the provided reference value. The term “substantially less”, when used in conjunction with a reference value describing a property or condition, refers to a value that is at least 1%, optionally at least 5%, optionally at least 10%, or optionally at least 20% less than the provided reference value.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

The terms “treating” or “treatment” as used herein, refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating,” and conjugations thereof, include prevention of an injury, pathology, condition, or disease.

The term “effective amount” as used herein, refers to an amount sufficient to accomplish a stated purpose (e.g. Achieve the effect for which it is administered, treat a disease, reduce one or more symptoms of a disease or condition, and the like). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “administering” as used herein, refers to oral administration, administration as an inhaled aerosol or as an inhaled dry powder, suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer”it is meant that a composition described herein is administered at the same time, just prior to, or just after the administration of one or more additional therapies, for example cancer therapies such as chemotherapy, hormonal therapy, radiotherapy, or immunotherapy. The compound of the invention can be administered alone or can be co-administered to the patient. Co-administration is meant to include simultaneous or sequential administration of the compound individually or in combination (more than one compound or agent). The compositions of the present invention can be delivered transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293306, 1996; Chonn, Curr. Opin. Biotechnol. 6:698-708, 1995; Qstio, Am. J Hasp. Pharm. 46: 1576-1587, 1989).

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell or a patient.

The terms “analog” and “analogue” are used interchangeably and are used in accordance with their plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound, including isomers thereof. Accordingly, an analog is a compound that is similar or comparable in function and appearance but not in structure or origin to a reference compound.

Except where otherwise specified, the term “molecular weight” refers to an average molecular weight. Except where otherwise specified, the term “average molecular weight,” refers to number-average molecular weight. Number average molecular weight is defined as the total weight of a sample volume divided by the number of molecules within the sample. As is customary and well known in the art, peak average molecular weight and weight average molecular weight may also be used to characterize the molecular weight of the distribution of polymers within a sample.

The term “weight-average molecular weight” (M_{w}) refers to the average molecular weight defined as the sum of the products of the molecular weight of each polymer molecule (Mᵢ) multiplied by its weight fraction (wᵢ): M_{w}=ΣwᵢMᵢ. As is customary and well known in the art, peak average molecular weight and number average molecular weight may also be used to characterize the molecular weight of the distribution of polymers within a sample.

The term “wt. %” or “wt %” refers to a weight percent, or a mass fraction represented as a percentage by mass. The term “at. %” or “at %” refers to an atomic percent, or an atomic ratio represented as a percentage of a type of atom with respect to total atoms in a given matter, such as a molecule, compound, material, nanoparticle, polymer, dispersion, etc.

The term “oligomerization” refers to a chemical process of converting a monomer or a mixture of monomers into an oligomer. The term “oxidative oligomerization” refers to a chemical process of oligomerization that includes chemical oxidation of one or more monomers to form an oligomer. An oligomerization is a polymerization process, wherein an oligomer is formed as a result of the polymerization.

As used herein, the term “polymer” refers to a molecule composed of repeating structural units connected by covalent chemical bonds often characterized by a number of repeating units, also referred to as base units (e.g., greater than or equal to 2 base units). As used herein, a term “polymer” is inclusive of an “oligomer” (i.e., an oligomer is a polymer; i.e., a polymer is optionally an oligomer). An “oligomer” refers to a molecule composed of repeating structural units, also referred to as base units, connected by covalent chemical bonds often characterized by a number of repeating units less such that the oligomer is a low molecular weight polymer. Preferably, but not necessarily, for example, an oligomer has equal to or less than 100 repeating units. Preferably, but not necessarily, for example, an oligomer has a lower molecular weight less than or equal to 10,000 Da. Oligomers may be the polymerization product of one or more monomer precursors. Polymerization of one or more monomers, or monomer precursors, resulting in formation of an oligomer may be referred to as oligomerization. An oligomer optionally includes 100 or less, 50 or less, 15 or less, 12 or less, 10 or less, or 5 or less repeating units (or, “base units”). An oligomer may be characterized has having a molecular weight of 10,000 Da or less, 5,000 Da or less, 1,000 Da or less, 500 Da or less, or 200 Da or less. A dimer, a trimer, a tetramer, or a pentamer is an oligomer having two, three, four, or five, respectively, repeating units, or base units. Polymers can have, for example, greater than 100 repeating units. Polymers can have, for example, a high molecular weight, such as greater than 10,000 Da, in some embodiments greater than or equal to 50,000 Da or greater than or equal to 100,000 Da. The term polymer includes homopolymers, or polymers consisting essentially of a single repeating monomer subunit. The term polymer also includes copolymers which are formed when two or more different types of monomers are linked in the same polymer. Copolymers may comprise two or more monomer subunits, and include random, block, brush, brush block, alternating, segmented, grafted, tapered and other architectures. Useful polymers include organic polymers or inorganic polymers that may be in amorphous, semi-amorphous, crystalline or semi-crystalline states. Polymer side chains capable of cross linking polymers (e.g., physical cross linking) may be useful for some applications.

An “oligomer” refers to a molecule composed of repeating structural units, also referred to as base units, connected by covalent chemical bonds often characterized by a number of repeating units less than that of a polymer (e.g., equal to or less than 100 repeating units) and a lower molecular weights (e.g. less than or equal to 10,000 Da) than polymers. Oligomers may be the polymerization product of one or more monomer precursors. Polymerization of one or more monomers, or monomer precursors, resulting in formation of an oligomer may be referred to as oligomerization. An oligomer optionally includes 100 or less, 50 or less, 15 or less, 12 or less, 10 or less, or 5 or less repeating units (or, “base units”). An oligomer may be characterized has having a molecular weight of 10,000 Da or less, 5,000 Da or less, 1,000 Da or less, 500 Da or less, or 200 Da or less. A dimer, a trimer, a tetramer, or a pentamer is an oligomer having two, three, four, or five, respectively, repeating units, or base units.

As used herein, the term “group” may refer to a functional group of a chemical compound. Groups of the present compounds refer to an atom or a collection of atoms that are a part of the compound. Groups of the present invention may be attached to other atoms of the compound via one or more covalent bonds. Groups may also be characterized with respect to their valence state. The present invention includes groups characterized as monovalent, divalent, trivalent, etc. valence states.

The term “moiety” refers to a group, such as a functional group, of a chemical compound or molecule. A moiety is a collection of atoms that are part of the chemical compound or molecule. The present invention includes moieties characterized as monovalent, divalent, trivalent, etc. valence states. Generally, but not necessarily, a moiety comprises more than one functional group.

As used herein, the term “substituted” refers to a compound wherein one or more hydrogens is replaced by another functional group, provided that the designated atom's normal valence is not exceeded. An exemplary substituent includes, but is not limited to: a halogen or halide, an alkyl, a cycloalkyl, an aryl, a heteroaryl, an acyl, an alkoxy, an alkenyl, an alkynyl, an alkylaryl, an arylene, a heteroarylene, an alkenylene, a cycloalkenylene, an alkynylene, a hydroxyl (—OH), a carbonyl (RCOR′), a sulfide (e.g., RSR′), a phosphate (ROP(═O)(OH)₂), an azo (RNNR′), a cyanate (ROCN), an amine (e.g., primary, secondary, or tertiary), an imine (RC(═NH)R′), a nitrile (RCN), a pyridinyl (or pyridyl), a diamine, a triamine, an azide, a diimine, a triimine, an amide, a diimide, or an ether (ROR′); where each of R and R′ is independently a hydrogen or a substituted or unsubstituted alkyl group, aryl group, alkenyl group, or a combination of these. Optional substituent functional groups are also described below. In some embodiments, such as some of Aspects 1-39, the term substituted refers to a compound wherein each of more than one hydrogen is replaced by another functional group, such as a halogen group. For example, when the substituent is oxo (i.e., ═O), then two hydrogens on the atom are replaced. The substituent group can be any substituent group described herein. For example, substituent groups can include one or more of a hydroxyl, an amino (e.g., primary, secondary, or tertiary), an aldehyde, a carboxylic acid, an ester, an amide, a ketone, nitro, an urea, a guanidine, cyano, fluoroalkyl (e.g., trifluoromethane), halo (e.g., fluoro), aryl (e.g., phenyl), heterocyclyl or heterocyclic group (i.e., cyclic group, e.g., aromatic (e.g., heteroaryl) or non-aromatic where the cyclic group has one or more heteroatoms), oxo, or combinations thereof. Combinations of substituents and/or variables are permissible provided that the substitutions do not significantly adversely affect synthesis or use of the compound.

As used herein, the term “derivative” refers to a compound wherein an atom or functional group is replaced by another atom or functional group (e.g., a substituent function group as also described below), including, but not limited to: a hydrogen, a halogen or halide, an alkyl, a cycloalkyl, an aryl, a heteroaryl, an acyl, an alkoxy, an alkenyl, an alkynyl, an alkylaryl, an arylene, a heteroarylene, an alkenylene, a cycloalkenylene, an alkynylene, a hydroxyl (—OH), a carbonyl (RCOR′), a sulfide (e.g., RSR′), a phosphate (ROP(═O)(OH)₂), an azo (RNNR′), a cyanate (ROCN), an amine (e.g., primary, secondary, or tertiary), an imine (RC(═NH)R′), a nitrile (RCN), a pyridinyl (or pyridyl), a diamine, a triamine, an azide, a diimine, a triimine, an amide, a diimide, or an ether (ROR′); where each of R and R′ is independently a hydrogen or a substituted or unsubstituted alkyl group, aryl group, alkenyl group, or a combination of these. Optional substituent functional groups are also described below. Preferably, the term “derivative” refers to a compound wherein one or two atoms or functional groups are independently replaced by another atom or functional group. Optionally, the term derivative does not refer to or include replacement of a chalcogen atom (S, Se) that is a member of a heterocyclic group. Optionally, and unless otherwise stated, the term derivative does not refer to or include replacement of a chalcogen atom (S, Se) nor a N (nitrogen) where the chalcogen atom and the N are members same heterocyclic group. Optionally, but not necessarily, the term derivative does not include breaking a ring structure, replacement of a ring member, or removal of a ring member.

As is customary and well known in the art, hydrogen atoms in formula, are not always explicitly shown, for example, hydrogen atoms bonded to the carbon atoms of aromatic, heteroaromatic, and alicyclic rings are not always explicitly shown. The structures provided herein, for example in the context of the description of formula and schematics and structures in the drawings, are intended to convey to one of reasonable skill in the art the chemical composition of compounds of the methods and compositions of the invention, and as will be understood by one of skill in the art, the structures provided do not indicate the specific positions and/or orientations of atoms and the corresponding bond angles between atoms of these compounds.

As used herein, the terms “alkylene” and “alkylene group” are used synonymously and refer to a divalent group derived from an alkyl group as defined herein. The invention includes compounds having one or more alkylene groups. Alkylene groups in some compounds function as linking and/or spacer groups. Compounds of the invention may have substituted and/or unsubstituted C₁-C₂₀ alkylene, C₁-C₁₀ alkylene and C₁-C₅ alkylene groups, for example, as one or more linking groups (e.g. L¹-L⁶).

As used herein, the terms “cycloalkylene” and “cycloalkylene group” are used synonymously and refer to a divalent group derived from a cycloalkyl group as defined herein. The invention includes compounds having one or more cycloalkylene groups. Cycloalkyl groups in some compounds function as linking and/or spacer groups. Compounds of the invention may have substituted and/or unsubstituted C₃-C₂₀ cycloalkylene, C₃-C₁₀ cycloalkylene and C₃-C₅ cycloalkylene groups, for example, as one or more linking groups (e.g. L¹-L⁶).

As used herein, the terms “arylene” and “arylene group” are used synonymously and refer to a divalent group derived from an aryl group as defined herein. The invention includes compounds having one or more arylene groups. In some embodiments, such as some of Aspects 1-39, an arylene is a divalent group derived from an aryl group by removal of hydrogen atoms from two intra-ring carbon atoms of an aromatic ring of the aryl group. Arylene groups in some compounds function as linking and/or spacer groups. Arylene groups in some compounds function as chromophore, fluorophore, aromatic antenna, dye and/or imaging groups. Compounds of the invention include substituted and/or unsubstituted C₃-C₃₀ arylene, C₃-C₂₀ arylene, C₃-C₁₀ arylene and C₁-C₅ arylene groups, for example, as one or more linking groups (e.g. L¹-L⁶).

As used herein, the terms “heteroarylene” and “heteroarylene group” are used synonymously and refer to a divalent group derived from a heteroaryl group as defined herein. The invention includes compounds having one or more heteroarylene groups. In an embodiment, a heteroarylene is a divalent group derived from a heteroaryl group by removal of hydrogen atoms from two intra-ring carbon atoms or intra-ring nitrogen atoms of a heteroaromatic or aromatic ring of the heteroaryl group. Heteroarylene groups in some compounds function as linking and/or spacer groups. Heteroarylene groups in some compounds function as chromophore, aromatic antenna, fluorophore, dye and/or imaging groups. Compounds of the invention include substituted and/or unsubstituted C₃-C₃₀ heteroarylene, C₃-C₂₀ heteroarylene, C₁-C₁₀ heteroarylene and C₃-C₅ heteroarylene groups, for example, as one or more linking groups (e.g. L¹-L⁶).

As used herein, the terms “alkenylene” and “alkenylene group” are used synonymously and refer to a divalent group derived from an alkenyl group as defined herein. The invention includes compounds having one or more alkenylene groups. Alkenylene groups in some compounds function as linking and/or spacer groups. Compounds of the invention include substituted and/or unsubstituted C₂-C₂₀ alkenylene, C₂-C₁₀ alkenylene and C₂-C₅ alkenylene groups, for example, as one or more linking groups (e.g. L¹-L⁶).

As used herein, the terms “cylcoalkenylene” and “cylcoalkenylene group” are used synonymously and refer to a divalent group derived from a cylcoalkenyl group as defined herein. The invention includes compounds having one or more cylcoalkenylene groups. Cycloalkenylene groups in some compounds function as linking and/or spacer groups. Compounds of the invention include substituted and/or unsubstituted C₃-C₂₀ cylcoalkenylene, C₃-C₁₀ cylcoalkenylene and C₃-C₅ cylcoalkenylene groups, for example, as one or more linking groups (e.g. L¹-L⁶).

As used herein, the terms “alkynylene” and “alkynylene group” are used synonymously and refer to a divalent group derived from an alkynyl group as defined herein. The invention includes compounds having one or more alkynylene groups. Alkynylene groups in some compounds function as linking and/or spacer groups. Compounds of the invention include substituted and/or unsubstituted C₂-C₂₀ alkynylene, C₂-C₁₀ alkynylene and C₂-C₅ alkynylene groups, for example, as one or more linking groups (e.g. L¹-L⁶).

As used herein, the term “halo” refers to a halogen group such as a fluoro (—F), chloro (—Cl), bromo (—Br), iodo (—I) or astato (—At).

The term “heterocyclic” refers to ring structures containing at least one other kind of atom, in addition to carbon, in the ring. Examples of such heteroatoms include nitrogen, oxygen and sulfur. Heterocyclic rings include heterocyclic alicyclic rings and heterocyclic aromatic rings. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups. Atoms of heterocyclic rings can be bonded to a wide range of other atoms and functional groups, for example, provided as substituents.

The term “carbocyclic” refers to ring structures containing only carbon atoms in the ring. Carbon atoms of carbocyclic rings can be bonded to a wide range of other atoms and functional groups, for example, provided as substituents.

The term “alicyclic ring” refers to a ring, or plurality of fused rings, that is not an aromatic ring. Alicyclic rings include both carbocyclic and heterocyclic rings.

The term “aromatic ring” refers to a ring, or a plurality of fused rings, that includes at least one aromatic ring group. The term aromatic ring includes aromatic rings comprising carbon, hydrogen and heteroatoms. Aromatic ring includes carbocyclic and heterocyclic aromatic rings. Aromatic rings are components of aryl groups.

The term “fused ring” or “fused ring structure” refers to a plurality of alicyclic and/or aromatic rings provided in a fused ring configuration, such as fused rings that share at least two intra ring carbon atoms and/or heteroatoms.

As used herein, the term “alkoxyalkyl” refers to a substituent of the formula alkyl-O-alkyl.

As used herein, the term “polyhydroxyalkyl” refers to a substituent having from 2 to 12 carbon atoms and from 2 to 5 hydroxyl groups, such as the 2,3-dihydroxypropyl, 2,3,4-trihydroxybutyl or 2,3,4,5-tetrahydroxypentyl residue.

As used herein, the term “polyalkoxyalkyl” refers to a substituent of the formula alkyl-(alkoxy)n-alkoxy wherein n is an integer from 1 to 10, preferably 1 to 4, and more preferably for some embodiments 1 to 3.

Amino acids include glycine, alanine, valine, leucine, isoleucine, methionine, proline, phenylalanine, tryptophan, asparagine, glutamine, glycine, serine, threonine, serine, rhreonine, asparagine, glutamine, tyrosine, cysteine, lysine, arginine, histidine, aspartic acid and glutamic acid. As used herein, reference to “a side chain residue of a natural α-amino acid” specifically includes the side chains of the above-referenced amino acids. Peptides and peptide moieties, as used and described herein, comprise two or more amino acid groups connected via peptide bonds.

Amino acids and amino acid groups refer to naturally-occurring amino acids, unnatural (non-naturally occurring) amino acids, and/or combinations of these. Naturally-occurring amino acids are those encoded by the genetic code, as well as those amino acids that are later modified, e.g., hydroxyproline, μ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of a naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, methionine methyl sulfonium. “Amino acid mimetics” refer to chemical compounds that have a structure that is different from the general chemical structure of an amino acid, but that functions in a manner similar to a naturally-occurring amino acid. Amino acids may be referred to herein by either the commonly known three letter symbols or by the one-letter symbols recommended by the IUPAC-IUB Biochemical Nomenclature Commission.

The terms “monomer unit,” “repeating monomer unit,” “repeating unit,” and “polymerized monomer” can be used interchangeably and refer to a monomeric portion of a polymer described herein which is derived from or is a product of polymerization of one individual “monomer” or “polymerizable monomer.” Each individual monomer unit of a polymer is derived from or is a product of polymerization of one polymerizable monomer. Each individual “monomer unit” or “repeating unit” of a polymer comprises one (polymerized) polymer backbone group. For example, in a polymer that comprises monomer units X and Y arranged as X—Y—X—Y—X—Y—X—Y (where each X is identical to each other X and each Y is identical to each other Y), each X and each Y is independently can be referred to as a repeating unit or monomer unit.

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. The term cycloalkyl specifically refers to an alky group having a ring structure such as ring structure comprising 3-30 carbon atoms, optionally 3-20 carbon atoms and optionally 2-10 carbon atoms, including an alkyl group having one or more rings. Cycloalkyl groups include those having a 3-, 4-, 5-, 6-, 7-, 8-, 9- or 10-member carbon ring(s) and particularly those having a 3-, 4-, 5-, 6-, 7-, or 8-member ring(s). The carbon rings in cycloalkyl groups can also carry alkyl groups. Cycloalkyl groups can include bicyclic and tricycloalkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group that has been modified by linkage to oxygen and can be represented by the formula R—O and can also be referred to as an alkyl ether group. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups. As used herein MeO— refers to CH₃O—. Compositions of some embodiments of the invention comprise alkyl groups as terminating groups, such as polymer backbone terminating groups and/or polymer side chain terminating groups. Substituted alkyl groups may include substitution to incorporate one or more silyl groups, for example wherein one or more carbons are replaced by Si.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cycloalkenyl groups include those in which a double bond is in the ring or in an alkenyl group attached to a ring. The term cycloalkenyl specifically refers to an alkenyl group having a ring structure, including an alkenyl group having a 3-, 4-, 5-, 6-, 7-, 8-, 9- or 10-member carbon ring(s) and particularly those having a 3-, 4-, 5-, 6- or 7-member ring(s). The carbon rings in cycloalkenyl groups can also carry alkyl groups. Cycloalkenyl groups can include bicyclic and tricyclic alkenyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogen atoms replaced with one or more fluorine atoms. Compositions of some embodiments of the invention comprise alkenyl groups as terminating groups, such as polymer backbone terminating groups and/or polymer side chain terminating groups.

Aryl groups include groups having one or more 5-, 6-7-, or 8-member aromatic rings, including heterocyclic aromatic rings. The term heteroaryl specifically refers to aryl groups having at least one 5-, 6-7-, or 8-member heterocyclic aromatic rings. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those that are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein are provided in a covalently bonded configuration in the compounds of the invention at any suitable point of attachment. In embodiments, aryl groups contain between 5 and 30 carbon atoms. In embodiments, aryl groups contain one aromatic or heteroaromatic six-member ring and one or more additional five- or six-member aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents. Compositions of some embodiments of the invention comprise aryl groups as terminating groups, such as polymer backbone terminating groups and/or polymer side chain terminating groups.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl groups having one or more hydrogens replaced with one or more fluorine atoms, chlorine atoms, bromine atoms and/or iodine atoms. Compositions of some embodiments of the invention comprise arylalkyl groups as terminating groups, such as polymer backbone terminating groups and/or polymer side chain terminating groups.

As to any of the groups described herein which contain one or more substituents, it is understood that such groups do not contain any substitution or substitution patterns which are sterically impractical and/or synthetically non-feasible. In addition, the compounds of this invention include all stereochemical isomers arising from the substitution of these compounds. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for any alkyl, alkenyl and aryl group includes substitution with one or more of the following substituents, among others:
halogen, including fluorine, chlorine, bromine or iodine;
pseudohalides, including —CN;
—COOR where R is a hydrogen or an alkyl group or an aryl group and more specifically where R is a methyl, ethyl, propyl, butyl, or phenyl group all of which groups are optionally substituted;
—COR where R is a hydrogen or an alkyl group or an aryl group and more specifically where R is a methyl, ethyl, propyl, butyl, or phenyl group all of which groups are optionally substituted;
—CON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;
—OCON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;
—N(R)₂ where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring which can contain one or more double bonds and can contain one or more additional carbon atoms;
—SR, where R is hydrogen or an alkyl group or an aryl group and more specifically where R is hydrogen, methyl, ethyl, propyl, butyl, or a phenyl group, which are optionally substituted;
—SO₂R, or —SOR where R is an alkyl group or an aryl group and more specifically where R is a methyl, ethyl, propyl, butyl, or phenyl group, all of which are optionally substituted;
—OCOOR where R is an alkyl group or an aryl group;
—SO₂N(R)₂ where each R, independently of each other R, is a hydrogen, or an alkyl group, or an aryl group all of which are optionally substituted and wherein R and R can form a ring which can contain one or more double bonds and can contain one or more additional carbon atoms; and
—OR where R is H, an alkyl group, an aryl group, or an acyl group all of which are optionally substituted. In a particular example R can be an acyl yielding —OCOR″ where R″ is a hydrogen or an alkyl group or an aryl group and more specifically where R″ is methyl, ethyl, propyl, butyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

As to any of the above groups which contain one or more substituents, it is understood that such groups do not contain any substitution or substitution patterns which are sterically impractical and/or synthetically non-feasible.

Many of the molecules disclosed herein contain one or more ionizable groups. Ionizable groups include groups from which a proton can be removed (e.g., —COOH) or added (e.g., amines) and groups that can be quaternized (e.g., amines). All possible ionic forms of such molecules and salts thereof are intended to be included individually in the disclosure herein. With regard to salts of the compounds herein, one of ordinary skill in the art can select from among a wide variety of available counterions that are appropriate for preparation of salts of this invention for a given application. In specific applications, the selection of a given anion or cation for preparation of a salt can result in increased or decreased solubility of that salt.

The compounds of this invention can contain one or more chiral centers. Accordingly, this invention is intended to include racemic mixtures, diastereomers, enantiomers, tautomers and mixtures enriched in one or more stereoisomer. The scope of the invention as described and claimed encompasses the racemic forms of the compounds as well as the individual enantiomers and non-racemic mixtures thereof.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another. It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of one or more chemical moieties, one or more functional groups, one or more atoms, one or more ions, an unpaired electron, or one or more other chemical species to the represented molecule, compound, or chemical formula. For example, in the formula

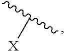

“X” represents a molecule or compound, the symbol “ [symbol] ” denotes a point of attachment of one or more chemical moieties, one or more functional groups, one or more atoms, one or more ions, an unpaired electron, or one or more other chemical species to X (where X corresponds to the represented molecule, compound, or chemical formula) via covalent bonding, wherein the covalent bonding can be any feasible covalent bond, including, but not limited to, a single bond, a double bond, or a triple bond. As an illustrative example, in the moiety

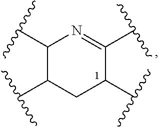 [SMILES: *C1=NC(*)C(*)CC1*]

the carbon labeled “1” has point of attachment which can be a double bond to another species, such a double bond to an oxygen, or two single bonds to two independent species, such as two distinct single bonds each to a hydrogen. As another illustrative example, when two points of attachment are shown on a single atom of a molecule, such as in the moiety

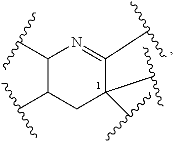 [SMILES: *C1=NC(*)C(*)CC1(*)*]

where the carbon labeled “1” has two points of attachment shown, the shown points of attachment on the same single atom (e.g., carbon 1), can be interpreted as representing either a preferable embodiment of two distinct bonds to that same single atom (e.g., two hydrogens bonded to carbon 1) or an optional embodiment of a single point of attachment to said same single atom (e.g., the two points of attachment on carbon 1 can optionally be consolidated as representing one double to carbon 1, such as a double bond to oxygen). As used herein, the various functional groups represented will be understood to have a point of attachment at the functional group having the hyphen or dash (-) or a dash used in combination with an asterisk (*). In other words, in the case of —CH₂CH₂CH₃ or —CH₂CH₂CH₃, it will be understood that the point of attachment is the CH₂ group at the far left. If a group is recited without an asterisk or a dash, then the attachment point is indicated by the plain and ordinary meaning of the recited group.

Where substituent groups are specified by their conventional chemical formulae, written from left to right, they equally encompass the chemically identical substituents that would result from writing the structure from right to left, e.g., —CH₂O— is equivalent to —OCH₂—.

The term “±” refers to an inclusive range of values, such that “X±Y,” wherein each of X and Y is independently a number, refers to an inclusive range of values selected from the range of X−Y to X+Y. In the cases of “X±Y” wherein Y is a percentage (e.g., 1.0±20%), the inclusive range of values is selected from the range of X−Z to X+Z, wherein Z is equal to X·(Y/100). For example, 1.0±20% refers to the inclusive range of values selected from the range of 0.8 to 1.2.

The term “and/or” is used herein, in the description and in the claims, to refer to a single element alone or any combination of elements from the list in which the term and/or appears. In other words, a listing of two or more elements having the term “and/or” is intended to cover embodiments having any of the individual elements alone or having any combination of the listed elements. For example, the phrase “element A and/or element B” is intended to cover embodiments having element A alone, having element B alone, or having both elements A and B taken together. For example, the phrase “element A, element B, and/or element C” is intended to cover embodiments having element A alone, having element B alone, having element C alone, having elements A and B taken together, having elements A and C taken together, having elements B and C taken together, or having elements A, B, and C taken together.

The term “melanin purity” can be used to characterize a collection or plurality of melanin materials (e.g., a plurality of artificial melanin nanoparticles, optionally in a dispersion or formulation) and refers to a relative measure of purity or content of a melanin type (e.g., pheomelanin) corresponding to a given melanin material with respect to all types (e.g., eumelanin, neuromelanin, pyomelanin, allomelanin and pheomelanin) of melanin materials in the collection or plurality of melanin materials. The term “pheomelanin purity” refers the relative purity or content of artificial melanin molecules and artificial melanin materials, or artificial melanin polymers or artificial melanin base units thereof, that are or comprise pheomelanin (in a collection or plurality of melanin or melanin-containing molecules or materials) with respect to all artificial melanin molecules and materials, or artificial melanin polymers or base units thereof, in said collection or plurality. Melanin purity is a quantitative value selected from the range of 0 to 1.

The term “precursor conversion efficiency” characterizes a method or synthesis of a melanin molecule or material and refers to a ratio of moles of produced selenomelanin monomers and selenomelanin base units to moles of a precursor used in said method of synthesis. The precursor refers to a first or a second precursor, such as a selenocystein or a eumelanin.

The term “polydispersity” or “dispersity” refers to a measure of heterogeneity of sizes particles. For example, polydispersity can be used to characterize a width of a particle size distribution (e.g., particle size vs. count or frequency), such as a particle size distribution of artificial melanin nanoparticles. For example, polydispersity may characterize heterogeneity of sizes of artificial melanin nanoparticles, such as artificial melanin nanoparticles in a solvent or artificial melanin nanoparticles in a dry state, such as those forming a film or layer. A “polydispersity index” is a measure of polydispersity. A polydispersity index can be measured using Dynamic Light Scattering (DLS), for example. Particles characterized by a polydispersity index of less than 0.1 are typically referred to as “monodisperse”. For example, a polydispersity index (PDI) can be calculated as the square of the standard deviation of the particle size distribution divided by its mean:

$$= ( \frac{σ}{d} ) ,$$

where σ is standard deviation of the particle size distribution and d is the mean diameter of the particle size distribution. Polydispersity and polydispersity index, as well as techniques for determining these, are further described in “NanoComposix's Guide to Dynamic Light Scattering Measurement and Analysis” [dated February 2015 (version 1.4), published by nanoComposix of San Diego, CA, and available at nanoComposix_Guidelines_for_DLS_Measurements_and_Analysis (last accessed Jun. 26, 2019)], which is incorporated herein by reference. The polydispersity index can also be calculated from electron microscope (SEM and/or TEM) images where the diameter is measured using software such as ImageJ, followed by calculating a mean size of the distribution, and then using the aforementioned equation to calculate the polydispersity index.

The term “sphericity” may be used to describe a given particle and refers to a ratio of surface area of a sphere (having the same volume as the given particle) to the surface area of the particle. An ideal sphere has a sphericity of 1. For example, an ideal cylinder has a sphericity of approximately 0.874.

The term “size stable” refers to stability of particles in a dispersion with respect to a size characteristic of said particles. Preferably, particles in a dispersion characterized as size stable are characterized by a size characteristic being within 50%, within 40%, within 30%, preferably within 20%, more preferably within 15%, still more preferably within 10%, further more preferably within 5%, or equivalent to a reference or initial size characteristic, under given conditions and optionally for a given time. For example, nanoparticles of a dispersion characterized as size-stable in the dispersion having a pH of at least 11, with respect to an average size of the nanoparticle in the dispersion having a pH of 7, have an average size in the pH 11 dispersion that is within 50%, within 40%, within 30%, preferably within 20%, more preferably within 15%, still more preferably within 10%, further more preferably within 5%, or equivalent to an average size of the otherwise equivalent nanoparticles in the otherwise equivalent dispersion having a pH of 7. Preferably, but not necessarily, nanoparticles characterized as size stable as so size stable for time that is at least 1 hour to 5 hours, preferably at least 5 hours to 12 hours, more preferably at least 12 hours to 1 week, still more preferably at least 1 week.

The terms “ultraviolet induced damage” and “UV induced damage” as used interchangeably herein refer, in the usual and customary sense, to chemical changes attending irradiation of light of sufficient energy. UV induced damage can include scission of nucleic acids (e.g., DNA or RNA), and breaking of bonds in proteins, lipids, and other physiological molecules. For example, the damage can be damage resulting from reactive oxygen species (ROS).

The term “self-assembly” refers to a process in which individual elements assemble into a network or organized structure without external direction. In an embodiment, self-assembly leads to a decrease in entropy of a system. In an embodiment, self-assembly may be induced, or initiated, via contacting or reacting the individual elements, optionally at a certain critical concentration, and/or via temperature and/or via pressure. A “self-assembled structure” is a structure or network formed by self-assembly. In an embodiment, self-assembly is a polymer crystallization process. The Gibbs free energy of the self-assembled structure is lower than of the sum of the individual components in their non-organized arrangement prior to self-assembly under otherwise identical conditions (e.g., temperature and pressure). In an embodiment, entropy of a self-assembled structure is lower than that of the sum of the individual components in their non-organized arrangement prior to self-assembly under otherwise identical conditions (e.g., temperature and pressure). For example, artificial melanin nanoparticles of this disclosure can form by self-assembly of a plurality of oligomers and/or melanin monomers. For example, structures or layers (e.g., films) for artificial melanin nanoparticles may form by self-assembly, such as structures or layers formed of artificial melanin nanoparticles and exhibiting structural color.

The terms “keratinocyte” and “keratinocytes” as used herein, refer to the predominant cell type in the epidermis, the outermost layer of the skin, constituting the majority (e.g., 90%-95%) of the cells found there. Keratinocytes are found in the deepest basal layer of the stratified epithelium that comprises the epidermis, and are sometimes referred to as basal cells or basal keratinocytes. Keratinocytes are maintained at various stages of differentiation in the epidermis and are responsible for forming tight junctions with the nerves of the skin. They also keep Langerhans cells of the epidermis and lymphocytes of the dermis in place. Keratinocytes contribute to protecting the body from UV radiation by taking up melanosomes. Keratinocytes contribute to protecting the body from UV radiation by taking up melanosomes, vesicles containing the endogenous photoprotectant melanin, from epidermal melanocytes. Each melanocyte in the epidermis has several dendrites that stretch out to connect it with many keratinocytes. The melanin is then stored within keratinocytes and melanocytes in the perinuclear area as “supranuclear caps”, where it protects the DNA from UV-induced damage. In embodiments, the terms “supranuclear cap” and “perinuclear cap” are used interchangeably and intended to have the same meaning. In addition to their structural role, keratinocytes play a role in immune system function. The skin is the first line of defense and keratinocytes serve as a barrier between an organism and its environment. In addition to preventing toxins and pathogens from entering an organisms body, they prevent the loss of moisture, heat and other important constituents of the body. In addition to their physical role, keratinocytes serve a chemical immune role as immunomodulaters, responsible for secreting inhibitory cytokines in the absence of injury and stimulating inflammation and activating Langerhans cells in response to injury. Langerhans cells serve as antigen-presenting cells when there is a skin infection and are the first cells to process microbial antigens entering the body from a skin breach.

The terms “under conditions suitable to afford uptake”, “taken up” and “take up” as used herein, refer, in the usual and customary sense, to experimental conditions well known in the art which allow uptake (e.g., endocytosis) of a species into a cell. In some embodiments, the term “internalized” when referring to particles internalized in or by a biological cell, refers to particles taken up by the biological cell, such as by, but not limited to, formation of perinuclear caps.

The term “endocytosis” as used herein, refers to a form of active transport in which a cell transports molecules (such as proteins) into the cell by engulfing them in an energy-using process. Endocytosis includes pinocytosis and phagocytosis. Pinocytosis is a mode of endocytosis in which small particles are brought into the cell, forming an invagination, and then suspended within small vesicles. These pinocytotic vesicles subsequently fuse with lysosomes to hydrolyze (break down) the particles. Phagocytosis is the process by which a cell engulfs a solid particle to form an internal compartment known as a phagosome.

In an embodiment, a composition or compound of the invention, such as an alloy or precursor to an alloy, is isolated or substantially purified. In an embodiment, an isolated or purified compound is at least partially isolated or substantially purified as would be understood in the art. In an embodiment, a substantially purified composition, compound or formulation of the invention has a chemical purity of 95%, optionally for some applications 99%, optionally for some applications 99.9%, optionally for some applications 99.99%, and optionally for some applications 99.999% pure.

### DETAILED DESCRIPTION OF THE INVENTION

In the following description, numerous specific details of the devices, device components and methods of the present invention are set forth in order to provide a thorough explanation of the precise nature of the invention. It will be apparent, however, to those of skill in the art that the invention can be practiced without these specific details.

Melanin has aroused scientific curiosity and attention for many years. Modern science has since revealed that melanin is an essential biopolymer across plant, animal, bacterial and fungal kingdoms with an astonishing array of functions, including coloration, camouflage, photoprotection and social communication. Nature makes melanin of different kinds: eumelanin, pheomelanin, allomelanin, neuromelanin and pyomelanin. Among them, eumelanin (black and brown pigment in dark hair) and pheomelanin (most widely known as the pigment in red hair) are the two basic forms, playing an essential role in human epidermal pigmentation. Artificial eumelanin has been synthesized by numerous methods. However, pheomelanin is far less studied because it is a crosslinked, insoluble material with high structural complexity and is never found in a pure form in nature.

Producing a high-fidelity chemical analogue of pheomelanin would be a powerful approach to studying these enigmatic materials. Such an approach would enable us to gain a deeper understanding of pheomelanin's intriguing properties. Underlining the need for new approaches is the fact that the existing structural and functional studies of pheomelanin have long conflicted with one another.

In this work, we investigate three different oxidative polymerization routes to generate synthetic pheomelanin, each giving rise to structurally dissimilar materials. Among them, the route employing 5-CD as a monomer was verified to yield a close analogue of extracted pheomelanin from humans and birds, as determined by parallel characterization (including solid-state Nuclear Magnetic Resonance and Electron Paramagnetic Resonance which has been especially elucidating) of pheomelanin extracted from the multiple biological sources. Although the covalent pathway for pheomelanin is elucidated by the Raper-Mason pathway, the non-covalent pathway has been relatively underappreciated, likely due to a lack of well-established model systems. With a good synthetic biomimetic material in hand, we discovered that cation-π interactions are an important driving force for pheomelanogenesis, further advancing our fundamental understanding of this important biological pigment. This study provides a route for manipulating artificial pheomelanin synthetically, and for driving our fundamental understanding of this biomaterial both on the molecular and supramolecular level.

Melanin is a pigment that is responsible for the color of human hair. Eumelanin, a brown and black pigment, is the most common form of melanin in humans. Pheomelanin, a red pigment, is also found in humans but is less common. The loss of melanin pigment in hair leads to hair whitening. Many commercial products have been developed to dye hair in a variety of colors including those not naturally occurring. However, these products typically use chemicals that are potentially carcinogenic and require harsh conditions, which can damage hair and cause allergic reactions. Commercial hair dye products also contain small molecules to dye the hair fibers, which are not naturally found in hair. Natural melanin in hair could be characterized through various techniques such as but not limited to solid state NMR and Fourier-Transform infrared spectroscopy and a synthetic version of melanin could be made that matches its exact chemical signature and color. This would allow for a customizable synthetic melanin dye unique to an individual's hair. Natural melanin is composed of various monomers which are then enzymatically oxidated. Monomers that mimic melanin such as dopamine, l-3,4-dihydroxyphenylalanine (L-DOPA), cystine, tyrosine, etc. could be polymerized through synthetic or enzymatic oxidation to synthesize a synthetic melanin hair dye. Depending on the chemical signature of the melanin in natural hair the combination of synthetic melanin monomers can be tuned to match the natural hair. This technique would engineer an exact melanin mimic biocompatible hair dye that would not have any of the adverse effects that are associated with commercial hair dyes.

Various techniques can be used to characterize a melanin content in an individual's hair. Such techniques include but are not limited to solid state NMR, Fourier-Transform infrared spectroscopy, mass spectrometry, matrix-assisted laser desorption/ionization, transmission electron microscopy, and scanning electron microscopy. Using various monomers found in natural melanin such as dopamine, l-3,4-dihydroxyphenylalanine (L-DOPA), cystine, tyrosine, etc. a synthetic melanin could be synthesized that chemically matches the natural melanin as seen through the various characterization techniques. Different oxidation techniques could also be used to polymerize the various melanin monomers such as ammonium hydroxide, sodium hydroxide, or chemoenzymatic oxidation using tyrosinase.

Hair dye is a commonly used product. Most of the time it used to cover up greying hair, but commercial hair dye often contains potentially toxic chemicals and requires harsh conditions. The color of natural human hair is due to a mixture of brown and black eumelanin, and red pheomelanin. Through the chemical analysis of the melanic composition in natural hair, a natural hair dye can be recreated to match both the color and chemical structure. This synthetic melanin hair dye would be as similar as possible to the natural hair without the use of harsh chemicals or conditions.

The synthesis of the synthetic melanin hair dye would be tunable and individualized for a person's natural hair. Our method of mimicking the chemical structural and color of melanin in the natural hair will be cost effective as it uses commercially available starting materials. It will also be biocompatible and will not require harsh or toxic chemicals or conditions as many commercial hair dyes do. It will also be metal-free.

Aspects of the methods described herein have or provide advantages including, but not limited to, a tunable chemical composition with variable chemical signature, color, and nutrients; mimics natural melanin optionally to give hair a natural look and feel; prepared from biocompatible materials; stable in aqueous conditions; prepared from inexpensive materials; metal-free; and prepared using minimal materials.

Certain Aspects

Various aspects are contemplated herein, several of which are set forth in the paragraphs below. It is explicitly contemplated and disclosed herein that any aspect or portion thereof can be combined to form an aspect. Moreover, for example, the term “any preceding aspect” means any aspect that appears prior to the aspect that contains such phrase is referenced (for example, the clause “Aspect 10: the method of any preceding aspect . . . ” means that any aspect prior to Aspect 10 is referenced, including Aspects 1-9). In addition, it is explicitly contemplated and disclosed herein that any reference to Aspect X, where X is an integer corresponding to one of the below Aspects (e.g., Aspect 11), includes reference to Aspects AXa, AXb, and/or AXc, if present, etc. (e.g., Aspect 11a, Aspect 11b, Aspect 11c, and/or Aspect 11d).

Aspect 1 provides a method for matching hair composition, the method comprising:
characterizing one or more first characteristics of a natural melanin composition of a hair sample from a subject; and
preparing a prepared artificial melanin formulation to approximate the one or more first characteristics;
   wherein the prepared artificial melanin formulation comprises one or more artificial melanin materials.

Aspect 2 provides the method of aspect 1, further comprising determining a theoretical artificial melanin formulation to approximate the one or more characteristics of the natural melanin composition;
wherein the step of preparing comprises preparing the prepared artificial melanin formulation according to the theoretical artificial melanin composition.

Aspect 3 provides the method of any one of the preceding aspects, wherein the step of characterizing comprises analyzing the hair sample using at least one technique selected from the group consisting of:
optical absorption spectroscopy, Fourier transform infrared (FTIR) spectroscopy, nuclear magnetic resonance (NMR) spectroscopy (e.g., solid state NMR (ssNMR) and heteronuclear correlation (HETCOR), mass spectroscopy (MS), electrospray ionization mass spectroscopy (SI-MS), dynamic light scattering (DLS), Zeta potential, electron microscope, scanning electron microscopy (SEM), energy dispersive spectroscopy (EDS), Raman spectroscopy, electron paramagnetic resonance (EPR) spectroscopy, ultraviolet-visible spectroscopy (UV-Vis), x-ray photoelectron spectroscopy (XPS), thermogravimetric analysis (TGA), differential scanning calorimetry (DSC), matrix assisted laser desorption/ionization (MALDI), and any combination of these.

Aspect 4 provides the method of any one of the preceding aspects, wherein at least one of the one or more first characteristics of the natural melanin composition is selected from the group consisting of:
a concentration of one or more pheomelanins;
a concentration ratio of one or more pheomelanins relative to all melanin in the natural melanin composition;
a concentration and/or concentration ratio of one or more eumelanins;
a concentration ratio of one or more eumelanins relative to all melanin in the natural melanin composition;
a concentration and/or concentration ratio of one or more allomelanins;
a concentration ratio of one or more allomelanins relative to all melanin in the natural melanin composition;
chemical identity or formula of one or more pheomelanins, one or more eumelanins, and/or one or more allomelanins in the hair sample;
an optical absorption spectrum;
an FTIR spectrum;
an NMR spectrum;
a relative elemental composition with respect to two or more elements;
a mass spectrum;
a DLS spectrum;
a Zeta potential data set;
a Raman spectrum;
an EPR spectrum;
contact angle;
and any combination of these.

Aspect 5 provides the method of any one of the preceding aspects, wherein the step of preparing comprises synthesizing at least a portion of the one or more artificial melanin materials.

Aspect 6 provides the method of aspect 5, wherein the step of preparing further comprises mixing the one or more artificial melanin materials to form an artificial melanin mixture; wherein the prepared artificial melanin formulation comprises the artificial melanin mixture.

Aspect 7 provides the method of any one of the preceding aspects, wherein the prepared artificial melanin formulation is characterized by one or more third characteristics each being approximately equivalent to the respective first characteristic of the natural melanin composition.

Aspect 8 provides the method of aspect 7, wherein the one or more third characteristic (of the prepared melanin formulation) is within 10% error and/or has at least 70% spectral matching with the first characteristic (of the natural melanin composition).

Aspect 9 provides the method of any one of the preceding aspects, wherein the prepared artificial melanin formulation has the same color as the natural melanin composition or wherein color of hair treated with the prepared artificial melanin formulation has the same color as the natural melanin composition or the hair sample.

Aspect 10 provides the method of any one of aspects 2-9, wherein the theoretical artificial melanin formulation is characterized by one or more second characteristics each being approximately equivalent to the respective first characteristic of the natural melanin composition.

Aspect 11 provides the method of aspect 10, wherein the step of determining comprises determining one or more formulation design parameters of the theoretical artificial melanin formulation which result in the one or more second characteristics being approximately equivalent to the one or more first characteristics, respectively.

Aspect 12 provides the method of aspect 11, wherein the one or more formulation design parameters are selected from the group consisting of:
a desired concentration and/or desired concentration ratio of one or more artificial melanin materials characterized as pheomelanin;
a desired concentration and/or desired concentration ratio of one or more artificial melanin materials characterized as eumelanin;
a desired concentration and/or desired concentration ratio of one or more artificial melanin materials characterized as allomelanin;
degree of polymerization of the melanin;
one or more desired size characteristics of the one or more artificial melanin materials;
one or more desired structural characteristics of the one or more artificial melanin materials;
one or more desired optical characteristics of the one or more artificial melanin materials;
one or more desired radical quenching characteristics of the one or more artificial melanin materials;
and any combination of these.

Aspect 13 provides the method of any one of aspect 10-12, wherein the one or more second characteristics (of the theoretical melanin formulation) is within 10% error and/or has at least 70% spectral matching with the first characteristic (of the natural melanin composition).

Aspect 14 provides the method of any one of the preceding aspects, wherein the theoretical and prepared artificial melanin compositions comprise one or more artificial eumelanins, one or more artificial allomelanins, one or more artificial pheomelanins, and a combination of these.

Aspect 15 provides the method of any one of the preceding aspects, wherein each of the one or more melanin materials is not bound to, conjugated to, attached to, coated by, encompassed by, or otherwise chemically associated with a natural or biological proteinaceous matrix, component, or lipid.

Aspect 16 provides the method of any one of the preceding aspects, wherein at least a portion of the one or more artificial melanin materials is characterized as eumelanin, pheomelanin, allomelanin, or a combination of these.

Aspect 17 provides the method of any one of the preceding aspects, wherein the one or more artificial melanin materials comprise a porous artificial melanin material.

Aspect 18 provides the method of any one of the preceding aspects, wherein at least a portion of the one or more artificial melanin materials comprises a plurality of melanin polymers; and wherein each melanin polymer comprises a plurality of covalently-bonded melanin base units.

Aspect 19 provides the method of aspect 18, wherein said melanin base units are one or more substituted or unsubstituted catechol-based monomer units, substituted or unsubstituted polyol-based monomer units, substituted or unsubstituted phenol-based monomer units, substituted or unsubstituted indole-based monomer units, substituted or unsubstituted benzothiazine-based monomer units, substituted or unsubstituted benzothiazole-based monomer units, substituted or unsubstituted dopamine-based monomer units, or any combination of these.

Aspect 20 provides the method of any one of aspects 18 or 19, wherein at least a portion of said melanin base units each independently comprises substituted or unsubstituted naphthalene.

Aspect 21 provides the method of any one of aspects 18-20, wherein at least a portion of the one or more artificial melanin materials comprises at least one dihydoxyindole (DHI) (e.g., 5,6-dihydroxyindole), at least one dihydroxyindole-2-carboxylic acid (DHICA) (e.g., 5,6-dihydroxyindole-2-carboxylic acid), or a combination of these.

Aspect 22 provides the method of any one of aspects 18-21, wherein at least 50% of the plurality of melanin polymers are selected from the group consisting of dimers, trimers, tetramers, pentamers, and any combination thereof.

Aspect 23 provides the method of any one of aspects 18-22, wherein each melanin oligomer is non-covalently associated with at least one other melanin oligomer or a melanin monomer via at least one of hydrogen bonding and π-π stacking of naphthalene rings; wherein the melanin monomer comprises the melanin base unit.

Aspect 24 provides the method of any one of aspects 18-23, wherein the artificial melanin material comprises a porous artificial melanin material; and wherein the melanin oligomers and/or polymers of the porous artificial melanin material are arranged to form an internal structure having a plurality of pores; wherein the porous artificial melanin material is characterized by a pore volume per mass of material greater than or equal to 0.1 cm³/g and wherein at least a portion of said pores have at least one size dimension greater than or equal to 0.5 nm.

Aspect 25 provides the method of any one of the preceding aspects, wherein at least a portion of the one or more artificial melanin materials comprises at least one substituted or unsubstituted benzothiazine, at least one substituted or unsubstituted benzothiazole, at least one substituted or unsubstituted benzoselenazole, at least one substituted or unsubstituted benzoselenazine, at least one derivative of any of these, or any combination of these.

Aspect 26 provides the method of any one of the preceding aspects, wherein at least a portion of the artificial melanin material comprises one or more selenomelanin polymers; wherein the one or more selenomelanin polymers comprise a plurality of covalently bonded selenomelanin base units; and wherein a chemical formula of each of the one or more selenomelanin base units comprises at least one selenium atom.

Aspect 27 provides the method of aspect 26, wherein each selenomelanin polymer is a pheomelanin.

Aspect 28 provides the method of any one of the preceding aspects, wherein at least a portion of the artificial melanin material comprises cation-π interactions.

Aspect 29 provides the method of any one of the preceding aspects, wherein the one or more artificial melanin materials comprise artificial melanin nanoparticles.

Aspect 30 provides the method of aspect 29, wherein the artificial melanin nanoparticles comprise porous artificial melanin nanoparticles.

Aspect 31 provides the method of aspect 29 or 30, wherein each of the one or more artificial melanin nanoparticles is not bound to, conjugated to, attached to, coated by, encompassed by, or otherwise chemically associated with a natural or biological proteinaceous matrix, component, or lipid.

Aspect 32 provides the method of any one of the preceding aspects, wherein the subject is a human or animal.

Aspect 33 provides the method of any one of the preceding aspects, wherein the hair samples is human hair.

Aspect 34 provides the method of any one of the preceding aspects, wherein the step of characterizing comprises extracting the natural melanin composition from the hair sample.

Aspect 35 provides the method of aspect 34, wherein the step of extracting is performed via chemical extraction, enzymatic extraction, or a combination of these.

Aspect 36 provides the method of any one of the preceding aspects, wherein the prepared artificial melanin formulation is provided to an end-user.

Aspect 37 provides the method of any one of the preceding aspects, wherein the end-user is an individual customer or a hair treatment facility.

Aspect 38 provides the method of any one of the preceding aspects further comprising treating hair of the same or different subject with the prepared artificial melanin formulation.

Aspect 39 provides a method for matching hair composition, the method comprising:
characterizing one or more first characteristics of a natural melanin composition of a hair sample from a subject,
determining a theoretical artificial melanin formulation to approximate the one or more characteristics of the natural melanin composition;
preparing a prepared artificial melanin formulation according to the theoretical artificial melanin composition;
wherein the prepared artificial melanin formulation comprises one or more artificial melanin materials.

Additional Descriptions

Various potentially useful descriptions, background information, applications/uses of embodiments herein, terminology (to the extent not inconsistent with the terms as defined herein), mechanisms, compositions, methods, definitions, and/or other embodiments may optionally be found in: International Patent App. No. PCT/US2017/041596 (published as International Pat. Pub. No. WO2018013609A2), International Patent App. No. PCT/US2020/039769 (published as International Pat. Pub. No. WO2021021350A3), International Patent App. No. PCT/US2020/057902 (published as International Pat. Pub. No. WO2021087076A1), and International Patent App. No. PCT/US2020/057939 (published as International Pat. Pub. No. WO2021096692A1), each of which is incorporated herein by reference to the extent not inconsistent herewith.

Optionally in some embodiments, such as some of Aspects 1-39, the artificial melanin material comprises synthetic melanin particles, also referred to herein as artificial melanin particles, also referred to interchangeably herein as artificial melanin-like particles or synthetic melanin-like particles, prepared by spontaneous oxidation of melanin monomers in an aqueous solution under alkaline conditions, to produce biocompatible, synthetic analogues of naturally occurring melanosomes.

Optionally in some embodiments, such as some of Aspects 1-39, such as some of Aspects 1-39, the artificial melanin material comprises synthetic melanin particles comprising non-natural particles composed of (e.g. comprising, consisting of, or consisting essentially of) melanin that is not bound to, conjugated to, attached to, coated by, encompassed by or otherwise associated with a lipid (i.e. a lipid comprising one or more proteins such as the lipid (plasma) membrane of a melanocyte or melanosome). Optionally in some embodiments, such as some of Aspects 1-39, the artificial melanin material comprises synthetic melanin particles comprising non-natural particles composed of (e.g. consisting of or consisting essentially of) melanin that is not bound to, conjugated to, attached to, coated by, encompassed by or otherwise associated with a proteinaceous lipid (i.e. a lipid comprising one or more proteins such as the lipid (plasma) membrane of a melanocyte or melanosome).

Optionally in some embodiments, such as some of Aspects 1-39, the artificial melanin material comprises synthetic melanin particles comprising melanin polymer being a fused ring melanin polymer which includes (e.g. consists of or consists essentially of) monomers of fused ring heteroaryl monomer and/or fused ring heterocycloalkyl monomers. Optionally in some embodiments, such as some of Aspects 1-39, the artificial melanin material comprises synthetic melanin particles comprising melanin polymer being a fused ring metal-binding melanin polymer comprising a melanin polymer bound to a plurality of transitions metals including but not limited to iron. Optionally in some embodiments, such as some of Aspects 1-39, the artificial melanin material comprises synthetic melanin particles comprising a fused ring melanin polymer being a dopamine monomer, including but not limited to dihydoxydopamine, 3,4-dihydoxydopamine, dioxydpoamine and/or 3,4-dioxydopamine. Optionally in some embodiments, such as some of Aspects 1-39, each of a fused ring heteroaryl monomer and/or fused ring heterocycloalkyl monomer may be substituted with one or more substituents selected from hydroxyl, carboxyl and/or oxy. Optionally in some embodiments, such as some of Aspects 1-39, each of the fused ring heteroaryl monomer is a 6,6-fused ring heteroaryl monomer, a 5,6-fused ring heteroaryl monomer or 6,5-fused ring heteroaryl monomer and each of the fused ring heterocycloalkyl moieties is a 6,6-fused ring heterocycloalkyl monomer, a 5,6-fused ring heterocycloalkyl 1 monomer or 6,5-fused ring heterocycloalkyl monomer. Optionally in some embodiments, such as some of Aspects 1-39, the fused ring heteroaryl monomers and/or fused ring heterocycloalkyl monomers (in the monovalent or bivalent form) are selected from indole (such as dihydroxyindole, 5,6-dihydroxyindole (DHI), 5,6-dihydroxyindole-2-carboxylic acid, dioxyindole, 5,6-dioxyindole, 5,6-droxyindole-2-carboxylic acid), benzothiazine, benzothiazole. Optionally in some embodiments, such as some of Aspects 1-39, the fused ring monomeric units of the fused ring melanin polymer are dihydroxy fused ring units (e.g. dihydroxy fused ring heteroaryl monomers and/or dihydroxy fused ring heterocycloalkyl monomers) wherein the hydroxy substituents are attached to adjacent carbons of a 6 membered ring (e.g. 6 membered carbon ring) of a fused ring monomer (also referred to herein as a “catechol fused ring monomer”). Optionally in some embodiments, such as some of Aspects 1-39, the fused ring melanin polymer may also contained oxidized versions of the dihydroxy fused ring units wherein one or both of the hydroxyl substituents are oxy substituents. Optionally in some embodiments, such as some of Aspects 1-39, the synthetic melanin particles can be in the form of a sphere, hollow sphere, nanorod, worm-like configuration, cylindrical configuration, and the like, with at least one dimensional axis thereof of from about 1 nm to about 1000 nm, from about 1 nm to about 1000 nm, from about 50 nm to about 500 nm, or from about 100 nm to about 300 nm, preferably with a high aspect ratio. Optionally in some embodiments, such as some of Aspects 1-39, synthetic melanin particles is in the form of a sphere of from about 50 nm to about 500 nm, from about 100 nm to about 300 nm, from about 150 nm to about 250 nm, or about 250 nm in average diameter. Optionally in some embodiments, such as some of Aspects 1-39, the synthetic melanin particles are in the form of a hollow sphere, optionally filled with silica. Optionally in some embodiments, such as some of Aspects 1-39, the synthetic melanin particles are capable of functioning as a pigment. Optionally in some embodiments, such as some of Aspects 1-39, the synthetic melanin particles are synthetic melanin nanoparticles.

Optionally in some embodiments, such as some of Aspects 1-39, an artificial melanin material disclosed herein comprises artificial melanin nanoparticles, wherein: each melanin nanoparticle of the plurality of artificial melanin nanoparticles comprises a plurality of melanin oligomers; each melanin oligomer comprises a plurality of covalently-bonded melanin base units; and each melanin base unit comprises substituted or unsubstituted naphthalene.

Optionally in some embodiments, such as some of Aspects 1-39, an artificial melanin material disclosed herein comprises artificial melanin nanoparticles, wherein: each melanin nanoparticle of the plurality of artificial melanin nanoparticles comprises a plurality of melanin oligomers; each melanin oligomer comprises a plurality of covalently-bonded melanin base units; and the plurality of artificial melanin nanoparticles are characterized by a peak size selected from the range of 100 nm to 300 nm and a polydispersity index selected to be less than or equal to 0.10, and optionally for some embodiments a polydispersity index selected to be less than or equal to 0.3 and optionally for some embodiments a polydispersity index selected to be less than or equal to 0.2. Optionally, the plurality of artificial melanin nanoparticles are characterized by a peak size selected from the range of 100 nm to 200 nm and a polydispersity index selected to be less than or equal to 0.10.

Optionally in some embodiments, such as some of Aspects 1-39, an artificial melanin material disclosed herein comprises artificial melanin nanoparticles, wherein: each melanin nanoparticle of the plurality of artificial melanin nanoparticles comprises a plurality of melanin oligomers; each melanin oligomer comprises a plurality of covalently-bonded melanin base units; and the plurality of artificial melanin nanoparticles exhibits structural color. Optionally, the plurality of artificial melanin nanoparticles exhibits structural color when the plurality of artificial melanin nanoparticles are in the form of a layer or film, such as a monolayer or thicker, or in the form of a pellet, such as a free-standing pellet, for example. Optionally, the plurality of artificial melanin nanoparticles exhibits structural color when the plurality of artificial melanin nanoparticles are in the form of a packed and/or ordered structure. Optionally, the plurality of artificial melanin nanoparticles exhibits structural color when the plurality of artificial melanin nanoparticles are dried or otherwise deposited onto a substrate.

Optionally in some embodiments, such as some of Aspects 1-39, an artificial melanin material disclosed herein comprises artificial melanin nanoparticles wherein: each melanin nanoparticle of the plurality of artificial melanin nanoparticles comprises a plurality of melanin oligomers; each melanin oligomer comprises a plurality of covalently-bonded melanin base units; and at least 50% of the plurality of melanin oligomers are selected from the group consisting of monomers, dimers, trimers, tetramers, pentamers, and any combination thereof. The monomers, dimers, trimers, tetramers, and pentamers have one, two, three, four, and five melanin base units, respectively. Optionally, at least 30%, optionally at least 40%, optionally at least 50%, optionally at least 60%, optionally at least 80%, of the plurality of melanin oligomers are selected from the group consisting of dimers, trimers, tetramers, pentamers, and any combination thereof, and the artificial melanin nanoparticles further comprise monomers. Optionally, at least 50% of the plurality of melanin oligomers are selected from the group consisting of dimers, trimers, tetramers, pentamers, and any combination thereof, and the artificial melanin nanoparticles further comprise monomers. Optionally, at least 30%, optionally at least 40%, optionally at least 50%, optionally at least 60%, optionally at least 80%, of the plurality of melanin oligomers are selected from the group consisting of dimers, trimers, tetramers, and any combination thereof, and the artificial melanin nanoparticles further comprise monomers. Optionally, at least 50% of the plurality of melanin oligomers are selected from the group consisting of dimers, trimers, tetramers, and any combination thereof, and the artificial melanin nanoparticles further comprise monomers. Optionally, at least 30% by mass, optionally at least 40% by mass, optionally at least 50% by mass, optionally at least 60% by mass, optionally at least 80% by mass, of each or of each of at least 80% of the plurality of artificial melanin nanoparticles is the monomers (each monomer having only one melanin base unit) and/or the melanin oligomers selected from the group consisting of dimers, trimers, tetramers, pentamers and any combination thereof. Optionally, at least 30% by mass, optionally at least 40% by mass, optionally at least 50% by mass, optionally at least 60% by mass, optionally at least 80% by mass, of each or of each of at least 80% of the plurality of artificial melanin nanoparticles is the monomers (each monomer having only one melanin base unit) and the melanin oligomers selected from the group consisting of dimers, trimers, tetramers, pentamers and any combination thereof. Optionally, at least 30% by mass, optionally at least 40% by mass, optionally at least 50% by mass, optionally at least 60% by mass, optionally at least 80% by mass, of each or of each of at least 80% of the plurality of artificial melanin nanoparticles is the monomers (each monomer having only one melanin base unit) and/or the melanin oligomers selected from the group consisting of dimers, trimers, tetramers, and any combination thereof. Optionally, at least 30% by mass, optionally at least 40% by mass, optionally at least 50% by mass, optionally at least 60% by mass, optionally at least 80% by mass, of each or of each of at least 80% of the plurality of artificial melanin nanoparticles is the monomers (each monomer having only one melanin base unit) and the melanin oligomers selected from the group consisting of dimers, trimers, tetramers, and any combination thereof.

Optionally in some embodiments, such as some of Aspects 1-39, an artificial melanin material disclosed herein comprises artificial melanin nanoparticles, wherein: each melanin nanoparticle of the plurality of artificial melanin nanoparticles comprises a plurality of melanin oligomers; each melanin oligomer comprises a plurality of covalently-bonded melanin base units; and each nanoparticle has a sphericity of less than 0.90 and has a shape characterized as at least one of: walnut-like, a collapsed sphere or collapsed ellipsoid, and a sphere or ellipsoid having a plurality of indentations.

Optionally in some embodiments, such as some of Aspects 1-39, an artificial melanin material disclosed herein comprises artificial melanin nanoparticles, wherein: each melanin nanoparticle of the plurality of artificial melanin nanoparticles comprises a plurality of melanin oligomers; each melanin oligomer comprises a plurality of covalently-bonded melanin base units; and the plurality of artificial melanin nanoparticles are characterized by a radical scavenging activity greater than that of polydopamine nanoparticles having the same diameter as the plurality of artificial melanin nanoparticles under otherwise identical condition. Optionally, the plurality of artificial melanin nanoparticles are characterized by a radical scavenging activity at least 5%, optionally at least 10%, optionally at least 15%, optionally at least 20%, greater than that of polydopamine nanoparticles having the same diameter as the plurality of artificial melanin nanoparticles under otherwise identical condition.

Optionally in some embodiments, such as some of Aspects 1-39, an artificial melanin material disclosed herein comprises artificial melanin nanoparticles, wherein: each melanin base unit comprises substituted or unsubstituted naphthalene. Optionally in some embodiments, such as some of Aspects 1-39, an artificial melanin material disclosed herein comprises artificial melanin nanoparticles, wherein: each melanin base unit comprises dihydroxynaphthalene. Optionally in some embodiments, such as some of Aspects 1-39, an artificial melanin material disclosed herein comprises artificial melanin nanoparticles, wherein: each melanin base unit comprises 1,8-dihydroxynaphthalene. According to certain embodiments, each melanin base unit comprises a structure having the formula FX1:

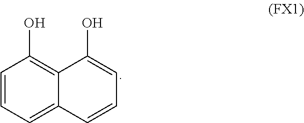 [SMILES: Oc1cccc2cccc(O)c12]

Optionally in some embodiments, such as some of Aspects 1-39, an artificial melanin material disclosed herein comprises artificial melanin nanoparticles, wherein: each melanin oligomer is free of nitrogen. According to certain embodiments, at least 20%, optionally at least 40%, optionally at least 50%, optionally at least 80% of the plurality of melanin oligomers are dimers having two covalently-bonded melanin base units. Optionally in some embodiments, such as some of Aspects 1-39, an artificial melanin material disclosed herein comprises artificial melanin nanoparticles, wherein: 20% to 80% of the plurality of melanin oligomers are dimers having two covalently-bonded melanin base units. Optionally in some embodiments, such as some of Aspects 1-39, an artificial melanin material disclosed herein comprises artificial melanin nanoparticles, wherein: at least 50% of the plurality of melanin oligomers are selected from the group consisting of monomers, dimers, trimers, tetramers, pentamers, and any combination thereof. The monomers, dimers, trimers, tetramers, and pentamers have one, two, three, four, and five melanin base units, respectively. Optionally, at least 30%, optionally at least 40%, optionally at least 50%, optionally at least 60%, optionally at least 80%, of the plurality of melanin oligomers are selected from the group consisting of dimers, trimers, tetramers, pentamers, and any combination thereof, and the artificial melanin nanoparticles further comprise monomers. Optionally in some embodiments, such as some of Aspects 1-39, an artificial melanin material disclosed herein comprises artificial melanin nanoparticles, wherein: at least 40% of the plurality of melanin oligomers are selected from the group consisting of monomers, dimers, trimers, tetramers, pentamers, and any combination thereof. Optionally in some embodiments, such as some of Aspects 1-39, an artificial melanin material disclosed herein comprises artificial melanin nanoparticles, wherein: at least 20%, optionally at least 40%, optionally at least 80%, of the plurality of melanin oligomers are selected from the group consisting of monomers, dimers, and trimers, and any combination thereof. Optionally in some embodiments, such as some of Aspects 1-39, an artificial melanin material disclosed herein comprises artificial melanin nanoparticles, wherein: at least 50% of the plurality of melanin oligomers are selected from the group consisting of monomers, dimers, and trimers, and any combination thereof. Optionally in some embodiments, such as some of Aspects 1-39, an artificial melanin material disclosed herein comprises artificial melanin nanoparticles, wherein: at least 30% by mass, optionally at least 40% by mass, optionally at least 50% by mass, optionally at least 60% by mass, optionally at least 80% by mass, of each or of each of at least 80% of the plurality of artificial melanin nanoparticles is the monomers (each monomer having only one melanin base unit) and/or the melanin oligomers selected from the group consisting of dimers, trimers, tetramers, pentamers and any combination thereof. Optionally in some embodiments, such as some of Aspects 1-39, an artificial melanin material disclosed herein comprises artificial melanin nanoparticles, wherein: at least 30% by mass, optionally at least 40% by mass, optionally at least 50% by mass, optionally at least 60% by mass, optionally at least 80% by mass, of each or of each of at least 80% of the plurality of artificial melanin nanoparticles is the monomers (each monomer having only one melanin base unit) and the melanin oligomers selected from the group consisting of dimers, trimers, tetramers, pentamers and any combination thereof. Optionally in some embodiments, such as some of Aspects 1-39, an artificial melanin material disclosed herein comprises artificial melanin nanoparticles, wherein: at least 30% by mass, optionally at least 40% by mass, optionally at least 50% by mass, optionally at least 60% by mass, optionally at least 80% by mass, of each or of each of at least 80% of the plurality of artificial melanin nanoparticles is the monomers (each monomer having only one melanin base unit) and/or the melanin oligomers selected from the group consisting of dimers, trimers, tetramers, and any combination thereof. Optionally in some embodiments, such as some of Aspects 1-39, an artificial melanin material disclosed herein comprises artificial melanin nanoparticles, wherein: at least 30% by mass, optionally at least 40% by mass, optionally at least 50% by mass, optionally at least 60% by mass, optionally at least 80% by mass, of each or of each of at least 80% of the plurality of artificial melanin nanoparticles is the monomers (each monomer having only one melanin base unit) and the melanin oligomers selected from the group consisting of dimers, trimers, tetramers, and any combination thereof. Optionally in some embodiments, such as some of Aspects 1-39, an artificial melanin material disclosed herein comprises artificial melanin nanoparticles, wherein: each melanin oligomer is non-covalently associated with at least one other melanin oligomer via at least one of hydrogen bonding and π-π stacking of naphthalene rings. Optionally in some embodiments, such as some of Aspects 1-39, an artificial melanin material disclosed herein comprises artificial melanin nanoparticles, wherein: each melanin oligomer is non-covalently associated with at least one other melanin oligomer or melanin monomer via at least one of hydrogen bonding and π-π stacking of naphthalene rings. Optionally in some embodiments, such as some of Aspects 1-39, an artificial melanin material disclosed herein comprises artificial melanin nanoparticles, wherein: a melanin monomer comprises the melanin base unit.

Optionally in some embodiments, such as some of Aspects 1-39, an artificial melanin material disclosed herein comprises artificial melanin nanoparticles, wherein: at least 50%, optionally at least 75%, optionally at least 90%, optionally at least 95%, of the plurality of nanoparticles is characterized by a sphericity of greater than 0.90. Optionally in some embodiments, such as some of Aspects 1-39, an artificial melanin material disclosed herein comprises artificial melanin nanoparticles, wherein: the plurality nanoparticles is characterized by a polydispersity index less than or equal to 0.10. Optionally in some embodiments, such as some of Aspects 1-39, an artificial melanin material disclosed herein comprises artificial melanin nanoparticles, wherein: each nanoparticle has a size characteristics, such as diameter, selected from the range of 10 nm to less than or equal to 1000 nm, optionally 100±50 nm to 300±50 nm. Optionally in some embodiments, such as some of Aspects 1-39, an artificial melanin material disclosed herein comprises artificial melanin nanoparticles, wherein: an average size characteristics, such as average diameter, of the artificial melanin nanoparticles is selected from the range of 10 nm to less than or equal to 1000 nm, optionally 20 nm to 500 nm, optionally 100 nm to 900 nm, optionally 200 nm to 900 nm, optionally 100 nm to 800 nm, optionally greater than 250 nm and less than 1000 nm. Optionally in some embodiments, such as some of Aspects 1-39, an artificial melanin material disclosed herein comprises artificial melanin nanoparticles, wherein: each of at least 55% (optionally at least 75%, optionally at least 80%, optionally at least 85%) of the nanoparticles has a size characteristic, such as diameter, selected from the range of greater than 200 nm, optionally greater than 250 nm, to less than 1000 nm. Optionally in some embodiments, such as some of Aspects 1-39, an artificial melanin material disclosed herein comprises artificial melanin nanoparticles, wherein: each nanoparticle has a size characteristics, such as diameter, selected from the range of 10 nm to less than or equal to 1000 nm, optionally 100 nm to 300 nm. Optionally in some embodiments, such as some of Aspects 1-39, an artificial melanin material disclosed herein comprises artificial melanin nanoparticles, wherein: each nanoparticle has a size characteristics, such as diameter, selected from the range of 20 nm to 300±50 nm. Optionally in some embodiments, such as some of Aspects 1-39, an artificial melanin material disclosed herein comprises artificial melanin nanoparticles, wherein: the plurality of artificial melanin nanoparticles are characterized by a peak size selected from the range of 10 nm to less than or equal to 1000 nm, optionally 100 nm to 300 nm. Optionally in some embodiments, such as some of Aspects 1-39, an artificial melanin material disclosed herein comprises artificial melanin nanoparticles, wherein: the plurality of artificial melanin nanoparticles are characterized by a peak size selected from the range of 10 nm to less than or equal to 1000 nm, optionally 100 nm to 200 nm. Optionally in some embodiments, such as some of Aspects 1-39, an artificial melanin material disclosed herein comprises artificial melanin nanoparticles, wherein: the plurality of artificial melanin nanoparticles are characterized by a peak size selected from the range of 50 nm to 300 nm, optionally 50 nm to 200 nm.

Optionally in some embodiments, such as some of Aspects 1-39, an artificial melanin material in the melanin formulation disclosed herein comprises artificial melanin nanoparticles, wherein the melanin formulation comprises a solvent or solvent mixture being at least 50% water, optionally at least 75% water, optionally at least 90% water, optionally at least 95%, by volume. According to certain embodiments, the solvent or solvent mixture comprises an organic solvent. According to certain embodiments, the solvent or solvent mixture comprises a buffer. According to certain embodiments, the organic solvent comprises methanol, ethanol, acetonitrile, acetone dichloromethane, dimethylformamide, ethyl acetate, acetone, or any combination thereof. In some embodiments, such as some of Aspects 1-39, artificial melanin nanoparticles are allowed to further age or further oxidize after synthesis. In some embodiments, such as some of Aspects 1-39, aging or further oxidation of the nanoparticles affects the solubility or dispersibility (in the melanin formulation), such as increasing stability in the presence of organic solvents. According to certain embodiments, the nanoparticles in the melanin formulation are characterized by a zeta potential or an average zeta potential selected from the range of −50 mV to −10 mV, optionally −40 to −20 mV, optionally in a solvent or solvent solution that is at least 95% water by volume. According to certain embodiments, the nanoparticles in the melanin formulation are stably dispersed without forming precipitates after at least 5 hours at a concentration selected from the range of 0.01 mg/mL to 5 mg/mL, optionally 0.01 mg/mL to 1 mg/mL, optionally within 20% of 0.1 mg/mL. According to certain embodiments, the nanoparticles in the melanin formulation are stably dispersed without forming precipitates after at least 12 hours at a concentration selected from the range of 0.01 mg/mL to 5 mg/mL, optionally 0.01 mg/mL to 1 mg/mL, optionally within 20% of 0.1 mg/mL.

Optionally in some embodiments, such as some of Aspects 1-39, an artificial melanin material disclosed herein comprises melanin monomers each melanin monomer having substituted or unsubstituted naphthalene. Optionally in some embodiments, such as some of Aspects 1-39, an artificial melanin material disclosed herein comprises melanin monomers each melanin monomer having dihydroxynaphthalene. Optionally in some embodiments, such as some of Aspects 1-39, an artificial melanin material disclosed herein comprises melanin monomers each melanin monomer having 1,8-dihydroxynaphthalene. Optionally in some embodiments, such as some of Aspects 1-39, an artificial melanin material disclosed herein comprises melanin monomers each melanin monomer being free of nitrogen. According to certain embodiments, the artificial melanin material is not derived or extracted from a biological source or a living organism.

Optionally in some embodiments, such as some of Aspects 1-39, an artificial melanin material disclosed herein or plurality of artificial melanin nanoparticles thereof is characterized by a radical scavenging activity greater than that of polydopamine nanoparticles having the same diameter as the plurality of artificial melanin nanoparticles under otherwise identical condition. Optionally in some embodiments, such as some of Aspects 1-39, an artificial melanin material disclosed herein or plurality of artificial melanin nanoparticles thereof is characterized by a radical scavenging activity at least 10%, optionally at least 15%, optionally at least 50%, greater than that of polydopamine nanoparticles having the same diameter as the plurality of artificial melanin nanoparticles under otherwise identical condition. Optionally in some embodiments, such as some of Aspects 1-39, an artificial melanin material disclosed herein or plurality of artificial melanin nanoparticles thereof is characterized by a radical scavenging activity of at least 0.012 mol/g using an assay of 2,2-diphenyl-1-(2,4,6-trinitrophenyl) hydrazyl (DPPH).

Optionally in some embodiments, such as some of Aspects 1-39, an artificial melanin material disclosed herein comprises one or more porous artificial melanin materials. Optionally in some embodiments, such as some of Aspects 1-39, an artificial melanin material disclosed herein comprises an porous artificial melanin material comprising: (i) one or more melanin oligomers, polymers or a combination thereof; wherein the one or more melanin oligomers and/or polymers comprise a plurality of covalently-bonded melanin base units; wherein the melanin oligomers and/or polymers are arranged to form an internal structure having a plurality of pores. Optionally in some embodiments, such as some of Aspects 1-39, an artificial melanin material disclosed herein comprises an porous artificial melanin material comprising: (i) one or more melanin oligomers, polymers or a combination thereof; wherein the one or more melanin oligomers and/or polymers comprise a plurality of covalently-bonded melanin base units; wherein the melanin oligomers and/or polymers are arranged to form an internal structure having a plurality of pores; wherein the porous artificial melanin material is characterized by a pore volume per mass of material greater than or equal to 0.1 cm³/g, optionally greater than or equal to 0.3 cm³/g, and wherein at least a portion of the pores have at least one size dimension, such as cross section dimension or longitudinal dimension, greater than or equal to 0.5 nm. Optionally in some embodiments, such as some of Aspects 1-39, an artificial melanin material disclosed herein comprises a porous artificial melanin material characterized by an average pore volume per mass of material selected from the range of 0.1 cm³/g to 0.6 cm³/g, and optionally 0.1 to 1 cm³/g and optionally 0.3 cm³/g to 0.6 cm³/g. Optionally in some embodiments, such as some of Aspects 1-39, an artificial melanin material disclosed herein comprises a porous artificial melanin material being a microporous material or a mesoporous material. Optionally in some embodiments, such as some of Aspects 1-39, an artificial melanin material disclosed herein comprises a porous artificial melanin material wherein the pores of the porous artificial melanin material include micropores each having at least one average size dimension, such as a cross sectional dimension and/or longitudinal dimension, selected from the range of 0.5 nm to 2.5 nm, and optionally 0.5 nm to 1.3 nm. Optionally in some embodiments, such as some of Aspects 1-39, an artificial melanin material disclosed herein comprises a porous artificial melanin material wherein the pores of the porous artificial melanin material include mesopores each having at least one average size dimension, such as a cross sectional dimension and/or longitudinal dimension, selected from the range of 2 nm to 50 nm, and optionally 2 nm to 25 nm. Optionally in some embodiments, such as some of Aspects 1-39, an artificial melanin material disclosed herein comprises a porous artificial melanin material wherein the pores are characterized by a distribution of pore sizes over the range of 0.5 nm to 50 nm. Optionally in some embodiments, such as some of Aspects 1-39, an artificial melanin material disclosed herein comprises a porous artificial melanin material wherein the pores of the internal structure are formed by organization of the melanin oligomers and/or polymers of the porous artificial melanin material. Optionally in some embodiments, such as some of Aspects 1-39, an artificial melanin material disclosed herein comprises a porous artificial melanin material wherein the pores of the internal structure are formed by close packing and/or self-assembly of the melanin oligomers and/or polymers of the porous artificial melanin material. Optionally in some embodiments, such as some of Aspects 1-39, an artificial melanin material disclosed herein comprises a porous artificial melanin material wherein the pores of the internal structure are formed by templating of the melanin oligomers and/or polymers of the porous artificial melanin material. Optionally in some embodiments, such as some of Aspects 1-39, an artificial melanin material disclosed herein comprises a porous artificial melanin material wherein the pores are not uniformly distributed throughout the porous melanin materials, for example, because the material is non-crystalline and/or amorphous. Optionally in some embodiments, such as some of Aspects 1-39, an artificial melanin material disclosed herein comprises a porous artificial melanin material wherein the porous artificial melanin material is an at least partially non-crystalline material and/or an amorphous material. Optionally in some embodiments, such as some of Aspects 1-39, an artificial melanin material disclosed herein comprises a porous artificial melanin material wherein the pores of the internal structure are randomly distributed. Optionally in some embodiments, such as some of Aspects 1-39, an artificial melanin material disclosed herein comprises a porous artificial melanin material wherein the pores of the internal structure are provided in repeating structures the amorphous porous artificial melanin material provided in an at least partial non-crystalline or amorphous state. Optionally in some embodiments, such as some of Aspects 1-39, an artificial melanin material disclosed herein comprises a porous artificial melanin material wherein the pores of porous artificial melanin material include one or more pore types selected from the group of cylindrical pores, channel-like pores, slit-shape pores, ink-bottle pores and any combination of these.

Optionally in some embodiments, such as some of Aspects 1-39, an artificial melanin material disclosed herein comprises a porous artificial melanin material having porous melanin particles, such as nanoparticles. Optionally in some embodiments, such as some of Aspects 1-39, an artificial melanin material disclosed herein comprises a porous artificial melanin material having porous melanin particles characterized by an average size selected from the range of 20 nm to 500 nm in diameter. Optionally in some embodiments, such as some of Aspects 1-39, an artificial melanin material disclosed herein comprises a porous artificial melanin material having porous melanin particles being one or more of solid particles, hollow particles, lacey particles, and any combinations of these. Optionally in some embodiments, such as some of Aspects 1-39, an artificial melanin material disclosed herein comprises a porous artificial melanin material having solid porous artificial melanin particles, for example, with pores distributed throughout the particle, for example uniformly distributed or randomly distributed, and without a hollow configuration. Optionally in some embodiments, such as some of Aspects 1-39, an artificial melanin material disclosed herein comprises a porous artificial melanin material having lacey porous artificial melanin particles, for example, with pores distributed throughout the particle, for example uniformly distributed or randomly distributed, and without a hollow configuration. Optionally in some embodiments, such as some of Aspects 1-39, an artificial melanin material disclosed herein comprises a porous artificial melanin material having hollow porous artificial melanin particles.

Optionally in some embodiments, such as some of Aspects 1-39, an artificial melanin material disclosed herein comprises a porous artificial melanin material having porous melanin particles that are purified or isolated.

Optionally in some embodiments, such as some of Aspects 1-39, an artificial melanin material disclosed herein comprises a porous artificial melanin material having melanin base units that are one or more substituted or unsubstituted catechol-based monomers, substituted or unsubstituted polyol-based monomers, substituted or unsubstituted phenol-based monomers, substituted or unsubstituted indole-based monomers, substituted or unsubstituted benzothiazine-based monomers, substituted or unsubstituted benzothiazole-based monomers, substituted or unsubstituted dopamine-based monomers or any combination of these.

Optionally in some embodiments, such as some of Aspects 1-39, an artificial melanin material disclosed herein comprises a porous artificial melanin material having or being allomelanin. In some embodiments, such as some of Aspects 1-39, for example, at least a portion of, and optionally all of, the melanin base units each independently comprises substituted or unsubstituted naphthalene. In some embodiments, such as some of Aspects 1-39, for example, at least a portion of, and optionally all of, the melanin base units each independently comprises dihydroxynaphthalene. In some embodiments, such as some of Aspects 1-39, for example, at least a portion of, and optionally all of, the melanin base units each independently comprises 1,8-dihydroxynaphthalene. In some embodiments, such as some of Aspects 1-39, for example, at least a portion of, and optionally all of, the melanin base units each independently comprises a structure having the formula FX1:

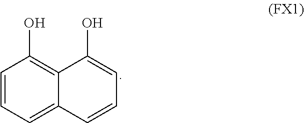 [SMILES: Oc1cccc2cccc(O)c12]

In some embodiments, such as some of Aspects 1-39, for example, each melanin oligomer is free of nitrogen.

Optionally in some embodiments, such as some of Aspects 1-39, an artificial melanin material disclosed herein comprises a porous artificial melanin material having polydopamine. In some embodiments, such as some of Aspects 1-39, for example, at least a portion of, and optionally all of, the melanin base units each independently comprises a substituted or unsubstituted dopamine monomer. In some embodiments, such as some of Aspects 1-39, for example, at least a portion of, and optionally all of, the melanin base units each independently are selected from the group consisting of substituted or unsubstituted dihydroxydopamine monomers, substituted or unsubstituted dioxydopamine monomers, substituted or unsubstituted dihydroxynaphthalene monomers, substituted or unsubstituted dioxydopamine monomers and any combination of these. In some embodiments, such as some of Aspects 1-39, for example, at least a portion of, and optionally all of, the melanin base units each independently are selected from the group consisting of 3,4-dihydroxydopamine monomers, 3,4-dioxydopamine monomers, 3,4-dihydroxynaphthalene monomers, and any combination of these.

Optionally in some embodiments, such as some of Aspects 1-39, an artificial melanin material disclosed herein comprises a porous artificial melanin material having allomelanin.

Optionally in some embodiments, such as some of Aspects 1-39, an artificial melanin material disclosed herein comprises substituted or unsubstituted catechol-based or polyol-based compounds. Optionally in some embodiments, such as some of Aspects 1-39, an artificial melanin material disclosed herein comprises substituted or unsubstituted dopamine monomers. Optionally in some embodiments, such as some of Aspects 1-39, an artificial melanin material disclosed herein comprises substituted or unsubstituted: dopamine monomers, 1,8-Dihydroxynaphthalene or its derivative, tyrosine monomers, tyramine monomers, amino acids, phenolamines, catecholamines, or any combination of these. Optionally in some embodiments, such as some of Aspects 1-39, an artificial melanin material disclosed herein comprises substituted or unsubstituted: dopamine monomers, tyrosine monomers, tyramine monomers, or a combination of these. Optionally in some embodiments, such as some of Aspects 1-39, an artificial melanin material disclosed herein is free of phenol derivatives, resorcinol, and/or paraphenylenediamine. Optionally, the dopamine monomers are selected from the group consisting of substituted or unsubstituted: dihydoxydopamine monomers, dihydoxydopamine dimers, dihydoxydopamine oligomers, dioxydopamine monomers, dioxydopamine dimers, dioxydopamine oligomers, dihydroxynapthalene monomers, dihydroxynapthalene dimers, dihydroxynapthalene oligomers, dioxydopamine monomers, dioxydopamine dimers, dioxydopamine oligomers, and any combination of these. Optionally, the dopamine monomers are selected from the group consisting of tyrosine and derivatives, phenol and derivatives, resorcinol and derivatives, and any combinations thereof. Optionally, the dopamine monomers are selected from the group consisting of phenol, resorcinol, L-DOPA, tyrosine and any combinations thereof. Optionally, the dopamine monomers are selected from the group consisting of cysteine derivatives, chalcogenides derivatives, selenocysteine, and any combinations thereof. Optionally in some embodiments, such as some of Aspects 1-39, an artificial melanin material disclosed herein comprises one or more monomers selected from the group consisting of:

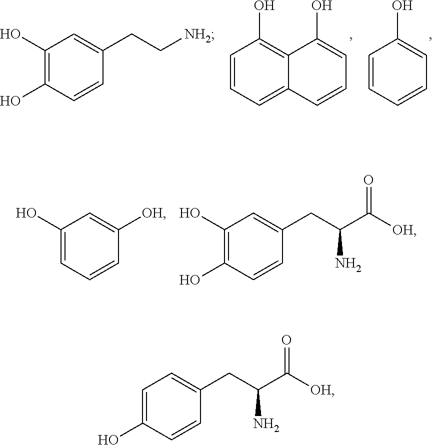

any combinations thereof, and any derivatives thereof. Optionally in some embodiments, such as some of Aspects 1-39, an artificial melanin material disclosed herein comprises one or more monomers having the formula (FX2):

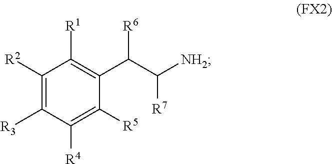

wherein one or more (optionally one, optionally two) of R¹-R⁷ is —OH and wherein each of the other of R¹-R⁷ is a functional group. Optionally, the each of the other of R¹-R⁷ is selected from the group consisting of hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —NR³⁹R⁴⁰, —NR⁴¹COR⁴², C₁-C₁₀ alkyl halide, acrylate, or catechol; wherein each of R³⁰-R⁴² is independently hydrogen, C₁-C₁₀ alkyl or C₅-C₁₀ aryl. Optionally, for any method disclosed herein, the artificial melanin precursors are one or more monomers having the formula (FX3):

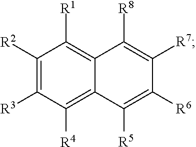

wherein one or more (optionally one, optionally two) of R¹-R⁸ is —OH and wherein each of the other of R¹-R⁸ is a functional group. Optionally, the each of the other of R¹-R⁷ is selected from the group consisting of hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —NR³⁹R⁴⁰, —NR⁴¹COR⁴², C₁-C₁₀ alkyl halide, acrylate, or catechol; wherein each of R³⁰-R⁴² is independently hydrogen, C₁-C₁₀ alkyl or C₅-C₁₀ aryl. Optionally in some embodiments, such as some of Aspects 1-39, an artificial melanin material disclosed herein comprises one or more thiol-reactive moieties. Optionally, the thiol-reactive moieties are one or more groups selected from the group consisting of a thiol, maleimide, pyridyl disulfide-based compound, alkene, alkyl halide and any combinations thereof. Optionally in some embodiments, such as some of Aspects 1-39, an artificial melanin material disclosed herein comprises one or more monomers having the formula (FX2) or (FX3), wherein one or more of R¹-R⁸ is a thiol-reactive moiety, such as a thiol, maleimide, pyridyl disulfide-based compound, alkene, alkyl halide and any combinations thereof.

Optionally in some embodiments, such as some of Aspects 1-39, an artificial melanin material disclosed herein comprises one or more artificial selenomelanin materials having: one or more selenomelanin polymers; wherein the one or more selenomelanin polymers comprise a plurality of covalently bonded selenomelanin base units; and wherein a chemical formula of each of the one or more selenomelanin base units comprises at least one selenium atom. Optionally, each selenomelanin polymer is a pheomelanin. Optionally, each of the selenomelanin monomers is an amino acid. Optionally, the chemical formula of each of the one or more selenomelanin base units comprises at least one covalent bond with each of the at least one selenium atom. Optionally, each of the one or more selenomelanin polymers is not bound to, conjugated to, attached to, coated by, encompassed by, or otherwise chemically associated with a natural or biological proteinaceous matrix, component, or lipid. Optionally, each of the plurality of selenomelanin base units is not bound to, conjugated to, attached to, coated by, encompassed by, or otherwise chemically associated with a natural or biological proteinaceous matrix, component, or lipid. Optionally, the chemical formula of each of the one or more selenomelanin base units comprises one selenium atom and two covalent bonds with the selenium atom. Optionally, the chemical formula of each of the one or more selenomelanin base units comprises a substituted or unsubstituted benzoselenazine or a derivative thereof, a substituted or unsubstituted benzoselenazole or a derivative thereof, a substituted or unsubstituted 7,10-dihydro-2H-[1,4]selenazino[3,2-h]isoquinolin-3(4H)-one or a derivative thereof, a substituted or unsubstituted benzoselenazinone or a derivative thereof, or any combination of these. Optionally, each of the one or more selenomelanin base units comprises a moiety characterized by formula FX11, FX12, FX13A, FX13B, FX14, a combination of any of these, or a derivative of any of these: (FX11);

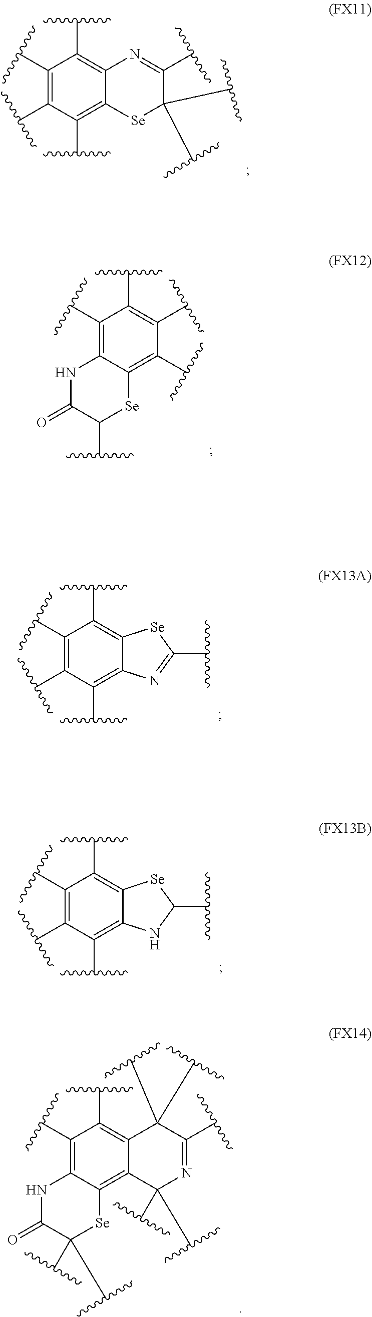 [SMILES: *C1=Nc2c(*)c(*)c(*)c(*)c2[Se]C1(*)* ; *c1c(*)c(*)c2c(c1*)NC(=O)C(*)[Se]2 ; *c1nc2c(*)c(*)c(*)c(*)c2[se]1 ; *c1c(*)c(*)c2c(c1*)NC(*)[Se]2 ; *C1=NC(*)(*)c2c3c(c(*)c(*)c2C1(*)*)NC(=O)C(*)(*)[Se]3]

Optionally, each of the one or more selenomelanin base units comprises a moiety characterized by formula FX11, FX12, FX13A, FX13B, FX14, or a combination of any of these. Optionally, each of the one or more selenomelanin base units comprises a moiety characterized by formula FX11, FX12, FX13A, FX13B, FX14, or a combination of any of these. Optionally, each of the one or more selenomelanin base units comprises a moiety characterized by formula FX11, FX12, FX13A, FX13B, or FX14. Optionally, each of the one or more selenomelanin base units comprises a moiety characterized by formula FX11. Optionally, an artificial selenomelanin material is one or a plurality of artificial selenomelanin nanoparticles, artificial selenomelanin layers, or artificial selenomelanin thin films. Optionally, an artificial selenomelanin material is one or a plurality of artificial selenomelanin nanoparticles. Optionally, each of the one or more selenomelanin base units comprises a heterocyclic moiety comprising a Se as a member of its ring structure. Optionally, each of the one or more selenomelanin base units comprises a heterocyclic moiety comprising a Se and a N as members of its ring structure. Optionally, each of the one or more selenomelanin base units comprises a moiety characterized by formula FX23, FX24, FX25, FX26, FX27, a derivative of any one of these, or a combination of any of these:

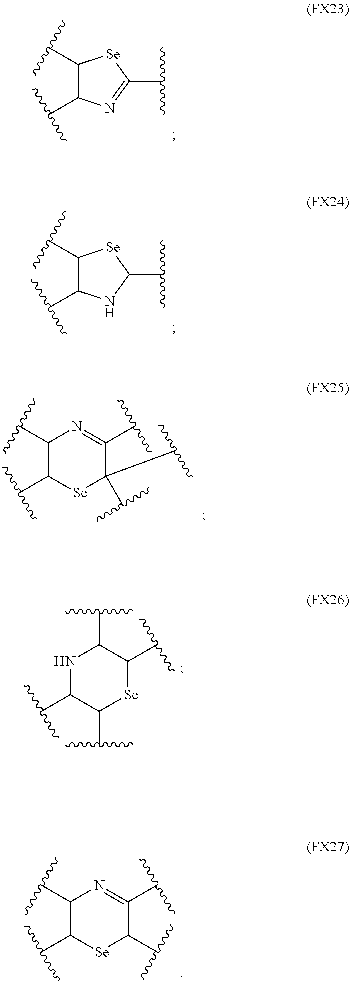 [SMILES: *C1=NC(*)C(*)[Se]1 ; *C1NC(*)C(*)[Se]1 ; *C1=NC(*)C(*)[Se]C1(*)* ; *C1NC(*)C(*)[Se]C1* ; *C1=NC(*)C(*)[Se]C1*]

Optionally, each of the one or more selenomelanin base units comprises a moiety characterized by formula FX23, FX24, FX25, FX26, FX27, or a combination of any of these. Optionally, each of the one or more selenomelanin base units comprises a moiety characterized by formula FX23, FX24, FX25, FX26, or FX27. Optionally, each of the selenomelanin monomers is characterized by formula FX15, FX16, FX17, FX18, FX19, FX20, or FX21:

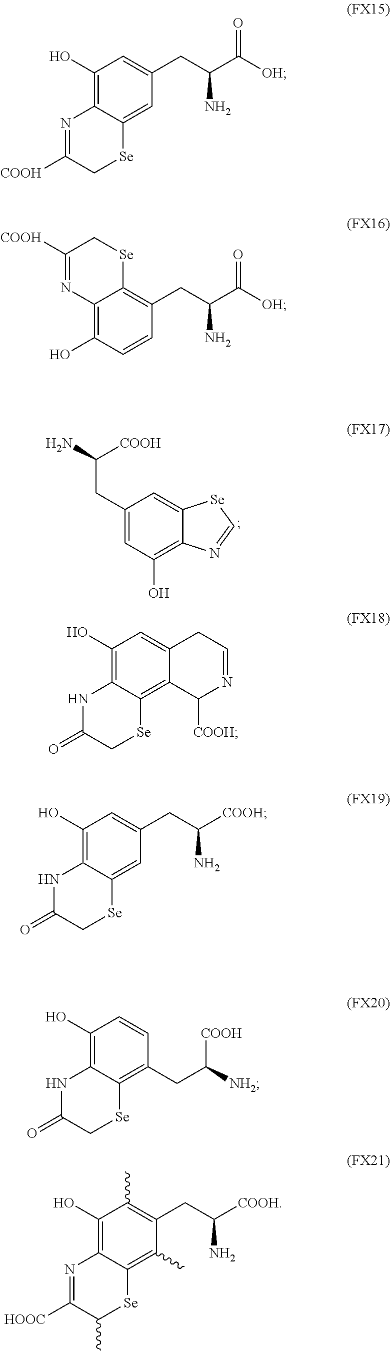

Optionally in some embodiments, such as some of Aspects 1-39, an artificial melanin material disclosed herein comprises one or more artificial selenomelanin materials wherein each of the one or more selenomelanin polymers is not bound to, conjugated to, attached to, coated by, encompassed by, or otherwise chemically associated with a natural or biological proteinaceous matrix, component, or lipid.

Optionally in some embodiments, such as some of Aspects 1-39, an artificial melanin material disclosed herein comprises one or more artificial selenomelanin materials wherein the chemical formula of each of the one or more selenomelanin base units comprises benzoselenazine and wherein the material comprises benzoselenazine at a concentration selected from the range of 10 wt. % to 100 wt. %. Optionally in some embodiments, such as some of Aspects 1-39, an artificial melanin material disclosed herein comprises one or more artificial selenomelanin materials having benzoselenazine at a concentration selected from the range of 50 wt. % to 60 wt. %. For example, the chemical formula of each of the one or more selenomelanin base units comprises benzoselenazine and the material can comprise benzoselenazine at a concentration of 55 wt. %. Optionally in some embodiments, such as some of Aspects 1-39, an artificial melanin material disclosed herein comprises one or more artificial selenomelanin materials characterized a concentration of selenium selected from the range of 2 wt. % to 23 wt. %. For example, the artificial selenomelanin material can be characterized a concentration of selenium of 12 wt. %.

Optionally in some embodiments, such as some of Aspects 1-39, an artificial melanin material in a melanin formulation disclosed herein comprises one or more artificial selenomelanin materials wherein the solvent or solvent mixture is at least 50% water. Optionally in some embodiments, such as some of Aspects 1-39, an artificial melanin material in a melanin formulation disclosed herein comprises one or more artificial selenomelanin materials having artificial selenomelanin nanoparticles characterized by an absolute value of a Zeta potential selected from the range of 15 mV to 50 mV, preferably 20 mV to 50 mV, optionally 15 mV to 40 mV, optionally 20 mV to 40 mV, optionally 15 mV to 30 mV, optionally 20 mV to 30 mV, optionally 17 mV to 34 mV. (The absolute value, or modulus, of a real number is the non-negative value of the real number without regard to its sign.) Optionally, the sign of the Zeta potential corresponding to the artificial selenomelanin nanoparticles in the artificial selenomelanin nanoparticle dispersion is negative. Optionally in some embodiments, such as some of Aspects 1-39, an artificial melanin material in a melanin formulation disclosed herein comprises one or more artificial selenomelanin materials having artificial selenomelanin nanoparticles being size-stable at nanoparticle concentrations selected from the range of 0.1 mg/mL to 104 mg/mL with respect to an average size of the nanoparticle at a concentration of 0.1 mg/mL. Optionally in some embodiments, such as some of Aspects 1-39, an artificial melanin material in a melanin formulation disclosed herein comprises one or more artificial selenomelanin materials having artificial selenomelanin nanoparticles being size-stable in the dispersion having a pH of 11, preferably at least 11, with respect to an average size of the nanoparticle in the dispersion having a pH of 7. Optionally in some embodiments, such as some of Aspects 1-39, an artificial melanin material in a melanin formulation disclosed herein comprises one or more artificial selenomelanin materials having artificial selenomelanin nanoparticles being size-stable when exposed to a concentration of NaCl selected from the range of 50 mM to 250 mM, preferably a concentration of NaCl being 250 mM, in the dispersion, with respect to an average size of the nanoparticles in an equivalent dispersion free of NaCl. Optionally in some embodiments, such as some of Aspects 1-39, an artificial melanin material in a melanin formulation disclosed herein comprises one or more artificial selenomelanin materials having artificial selenomelanin nanoparticles being stably dispersed in the dispersion for at least 7 days, preferably at least 14 days, preferably at least 60 days under ambient conditions.

Optionally in some embodiments, such as some of Aspects 1-39, an artificial melanin material in a melanin formulation disclosed herein comprises one or more artificial selenomelanin materials having artificial selenomelanin nanoparticles being characterized by a melanin purity of at least 20%, optionally at least 25%, optionally at least 30%, preferably at least 50%, more preferably at least 70%, further more preferably at least 80%, yet further more preferably at least 90%, more preferably for some applications at least 95%, still more preferably for some applications at least 99%, still further more preferably for some application at least 99.9%. Optionally in some embodiments, such as some of Aspects 1-39, an artificial melanin material in a melanin formulation disclosed herein comprises one or more artificial selenomelanin materials having artificial selenomelanin nanoparticles wherein each of at least 50%, optionally at least 75%, preferably at least 90%, more preferably at least 95%, further more preferably at least 99%, of the plurality of artificial melanin nanoparticles comprises a selenomelanin polymer having selenomelanin base units comprising a moiety characterized by formula FX11, FX12, FX13A, FX13B, FX14, or a combination of any of these:

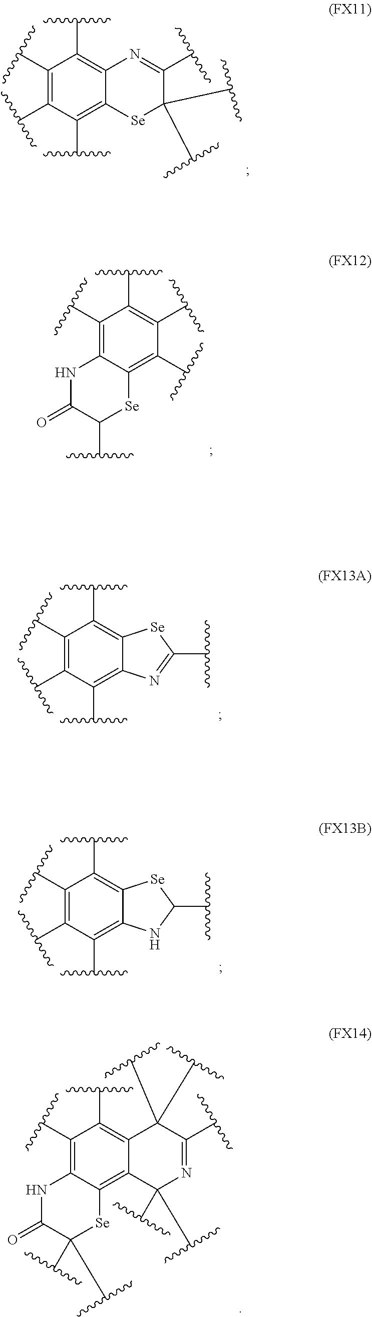 [SMILES: *C1=Nc2c(*)c(*)c(*)c(*)c2[Se]C1(*)* ; *c1c(*)c(*)c2c(c1*)NC(=O)C(*)[Se]2 ; *c1nc2c(*)c(*)c(*)c(*)c2[se]1 ; *c1c(*)c(*)c2c(c1*)NC(*)[Se]2 ; *C1=NC(*)(*)c2c3c(c(*)c(*)c2C1(*)*)NC(=O)C(*)(*)[Se]3]

Optionally in some embodiments, such as some of Aspects 1-39, an artificial melanin material in a melanin formulation disclosed herein comprises one or more artificial selenomelanin materials having artificial selenomelanin nanoparticles wherein each of at least 50%, optionally at least 75%, preferably at least 90%, more preferably at least 95%, further more preferably at least 99%, of the plurality of artificial melanin nanoparticles comprises a selenomelanin polymer having selenomelanin base units comprises a heterocyclic moiety comprising a Se as a member of its ring structure. Optionally in some embodiments, such as some of Aspects 1-39, an artificial melanin material in a melanin formulation disclosed herein comprises one or more artificial selenomelanin materials having artificial selenomelanin nanoparticles wherein each of at least 50%, optionally at least 75%, preferably at least 90%, more preferably at least 95%, further more preferably at least 99%, of the plurality of artificial melanin nanoparticles comprises a selenomelanin polymer having selenomelanin base units comprises a heterocyclic moiety comprising a Se and a N as members of its ring structure. Optionally in some embodiments, such as some of Aspects 1-39, an artificial melanin material in a melanin formulation disclosed herein comprises one or more artificial selenomelanin materials having artificial selenomelanin nanoparticles wherein each of at least 50%, optionally at least 75%, preferably at least 90%, more preferably at least 95%, further more preferably at least 99%, of the plurality of artificial melanin nanoparticles comprises a selenomelanin polymer having selenomelanin base units comprises a moiety characterized by formula FX23, FX24, FX25, FX26, FX27, a derivative of any one of these, or a combination of any of these. Optionally in some embodiments, such as some of Aspects 1-39, an artificial melanin material in a melanin formulation disclosed herein comprises one or more artificial selenomelanin materials having artificial selenomelanin nanoparticles wherein each of at least 50%, optionally at least 75%, preferably at least 90%, more preferably at least 95%, further more preferably at least 99%, of the plurality of artificial melanin nanoparticles comprises a selenomelanin polymer having selenomelanin base units comprises a moiety characterized by formula FX23, FX24, FX25, FX26, FX27, or a combination of any of these. Optionally in some embodiments, such as some of Aspects 1-39, an artificial melanin material in a melanin formulation disclosed herein comprises one or more artificial selenomelanin materials having artificial selenomelanin nanoparticles wherein each of at least 50%, optionally at least 75%, preferably at least 90%, more preferably at least 95%, further more preferably at least 99%, of the plurality of artificial melanin nanoparticles comprises a selenomelanin polymer having selenomelanin base units comprises a moiety characterized by formula FX23, FX24, FX25, FX26, or FX27. Optionally in some embodiments, such as some of Aspects 1-39, an artificial melanin material in a melanin formulation disclosed herein comprises one or more artificial selenomelanin materials having artificial selenomelanin nanoparticles wherein each of the one or more selenomelanin nanoparticles is not bound to, conjugated to, attached to, coated by, encompassed by, or otherwise chemically associated with a natural or biological proteinaceous matrix, component, or lipid. Optionally in some embodiments, such as some of Aspects 1-39, an artificial melanin material in a melanin formulation disclosed herein comprises one or more artificial selenomelanin materials having artificial selenomelanin nanoparticles wherein each of at least 50%, optionally at least 75%, preferably at least 80%, preferably at least 90%, more preferably at least 95%, further more preferably at least 99%, of the artificial selenomelanin nanoparticles is free of artificial melanin monomers. Optionally in some embodiments, such as some of Aspects 1-39, an artificial melanin material in a melanin formulation disclosed herein comprises one or more artificial selenomelanin materials having artificial selenomelanin nanoparticles wherein each of the artificial selenomelanin nanoparticles is free of artificial melanin monomers. For example, artificial selenomelanin materials, such as nanoparticles, are extensively washed with HCl solution (e.g., once) and pure water (e.g., 3 times), as a result of which the artificial selenomelanin materials, or dispersion or formulations thereof, can be free of artificial selenomelanin monomers, as characterized by solid-state NMR, UV-Vis spectra, etc. Optionally, a melanin formulation disclosed herein comprises a concentration of melanin monomers being less than IC50 of the monomers, respectively. Optionally in some embodiments, such as some of Aspects 1-39, an artificial melanin material in a melanin formulation disclosed herein comprises one or more artificial selenomelanin materials having artificial selenomelanin nanoparticles wherein each of the artificial selenomelanin nanoparticles is external (extracellular) of a biological cell.

The invention can be further understood by the following non-limiting examples.

### Example 1A: Biomimetic Pheomelanin to Unravel the Electronic, Molecular and Supramolecular Structure of the Natural Product

Herein, we investigate synthetic routes to a close mimic of natural pheomelanin. Three different oxidative polymerization routes were attempted to generate synthetic pheomelanin, each giving rise to structurally dissimilar materials. Among them, the route employing 5-cysteinyl-dihydroxyphenylalanine (5-CD) as a monomer was verified as a close analogue of extracted pheomelanin from humans and birds. The resulting biomimetic and natural pheomelanins were compared via various techniques, including solid-state Nuclear Magnetic Resonance (ssNMR) and Electron Paramagnetic Resonance (EPR). This synthetic pheomelanin closely mimics the structure of natural pheomelanin as determined by parallel characterization of pheomelanin extracted from multiple biological sources. With a good synthetic biomimetic material in hand, we describe cation-π interactions as an important driving force for pheomelanogenesis, further advancing our fundamental understanding of this important biological pigment.

Introduction

Melanin is an important class of polymeric biological pigment derived mainly from amino acid precursors.¹ Found across different kingdoms of life, melanins have a myriad of important functions, including coloration, camouflage, thermal regulation, photoprotection, and radioprotection.²⁻¹³ Epidermal pigmentation in human beings is considered to be composed of two separate but biosynthetically related melanins: eumelanin and pheomelanin.^{14, 15} Eumelanin is well-studied as the most important factor in protection from harmful solar UV radiation.^{5, 16, 17} While its cousin pheomelanin, which is prevalent in red hair and fair skin, is far less studied.^{18, 19} Although pheomelanin is generally thought to be phototoxic and potentially responsible for fair skinned people being more susceptible to sunburn and skin cancer,^{20, 21} from an evolutionary perspective, it must have played some beneficial role. In fact, several studies have shown that pheomelanin can provide better ionizing radiation protection than eumelanin.^{22, 23} Additionally, pheomelanogenesis was suggested to be related to Parkinson's disease (PD), as red hair color is associated with a significantly higher risk for PD.²⁴ However, other studies showed that loss of neuromelanin (a mixture of pheomelanin and eumelanin) and subsequent depigmentation of the substantia nigra is a hallmark feature of PD.^{25, 26} Undermining our complete understanding of the material is that structural and functional studies of pheomelanin have long conflicted with one another.^{23, 27, 28} Therefore, to better understand the functions of pheomelanin, detailed structural characterization is essential.

Natural pheomelanin is likely synthesized from L-dihydroxyphenylalanine (L-DOPA) and cysteine.^{14, 25} This biosynthesis is described by the Raper-Mason pathway (RMP), in which the nucleophilic addition of cysteine to enzymatically generated DOPA quinone forms 5-cysteinyl-DOPA (5-CD), 2-cysteinyl-DOPA (2-CD), 2,5-dicysteinyl-DOPA and trace amounts of 6-cysteinyl-DOPA (6-CD) as the subunits of pheomelanin. Further oxidation leads to more hierarchical pheomelanin pigments. As with eumelanin, pheomelanin presents a significant challenge for fundamental characterization, because it is a highly crosslinked, insoluble material with no sequence-controlled structure, unlike other common biopolymers such as proteins and DNA.²⁹ Indeed, studies have been largely hampered by ambiguity of chemical structures present in the material. One of the reasons is the inherent difficulty in isolating pure pheomelanin.¹⁴ This is not only because of the lipids and proteins found together with the pigment even after extensive attempts at purification, but also because pheomelanin covalently conjugates to and physically traps other biomolecules.^{30, 31} Therefore, producing a high-fidelity chemical analogue of pheomelanin can play an important role in understanding the biomaterial.¹² Previously, pheomelanin synthesis have been performed using a combination of _{L}-DOPA and cysteine as comonomers, or using the 5-CD heterodimer.^{19, 22, 27, 28, 32-34} However, pheomelanin materials synthesized using these two main routes have not been thoroughly characterized and compared against each other. Presumably, this lack of investigation stems from the aforementioned challenges.

Furthermore, although the primary structure is caused by covalent bond forming reactions, supramolecular interactions are important for the formation of melanin pigments because aggregation of the chromophores accounts for perturbations of the π-electron systems, thereby affecting color development.^{29, 35} The covalent pathway for pheomelanin is elucidated by the RMP,^{14, 16} but the non-covalent pathway has been relatively underappreciated, likely due to a lack of well-established model systems. The readily-accepted interactions in pheomelanin are hydrogen bonding and π-π stacking as demonstrated by eumelanin-based research.³⁶⁻³⁸ Recently, cation-π interactions have been shown to play a key role in the progressive assembly of polydopamine (PDA) type eumelanin.³⁹ Since nature has utilized cation-π interactions for various important biomacromolecules,⁴⁰ it is desirable to determine whether this important supramolecular interaction also exists in pheomelanin.

Here, we show the synthesis of artificial pheomelanin and directly compare it with multiple natural samples for verification and elucidation. Through comprehensive characterization, properties such as color and spectral signatures via ssNMR and EPR were identified and assigned to natural pheomelanin. This study provides a route for manipulating artificial pheomelanin synthetically, and for driving our fundamental understanding of this biomaterial both on the molecular and supramolecular level.

Results and Discussion

Chemical Synthesis of Pheomelanin

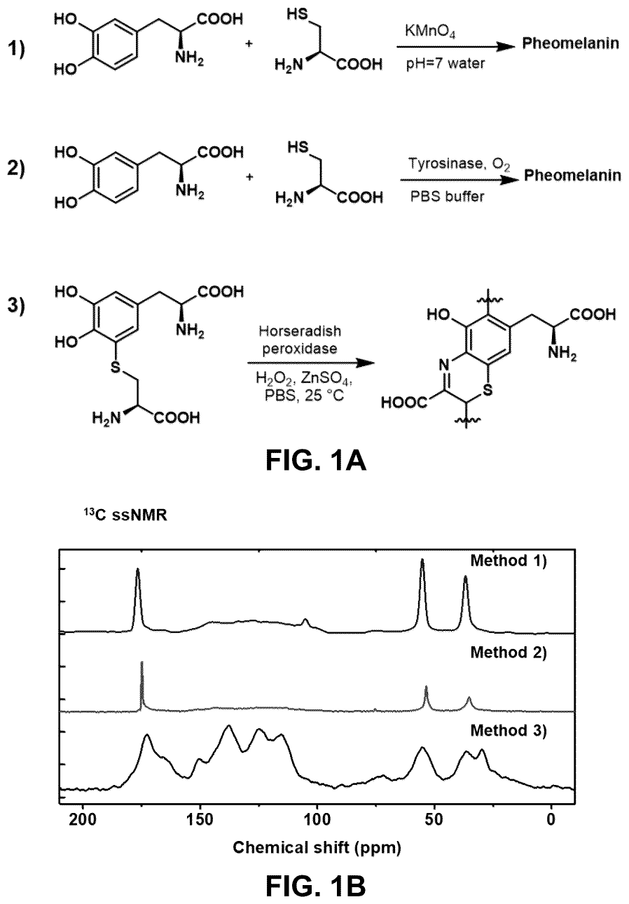

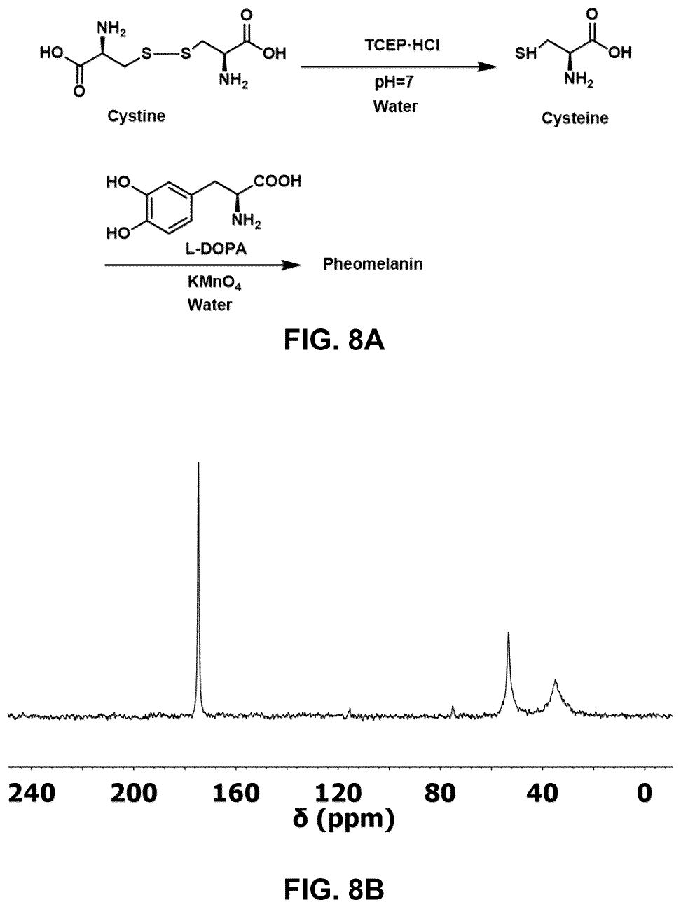

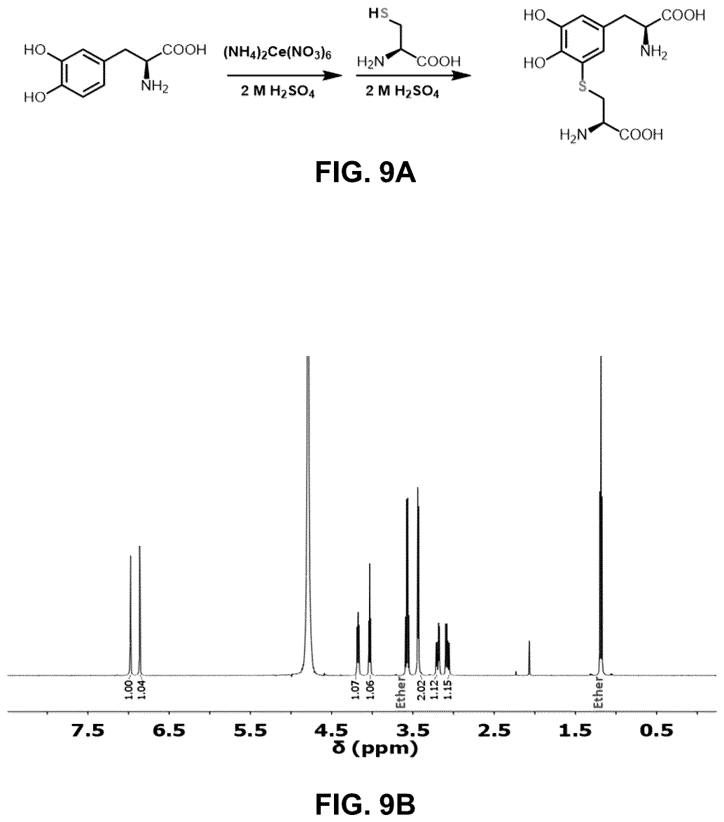

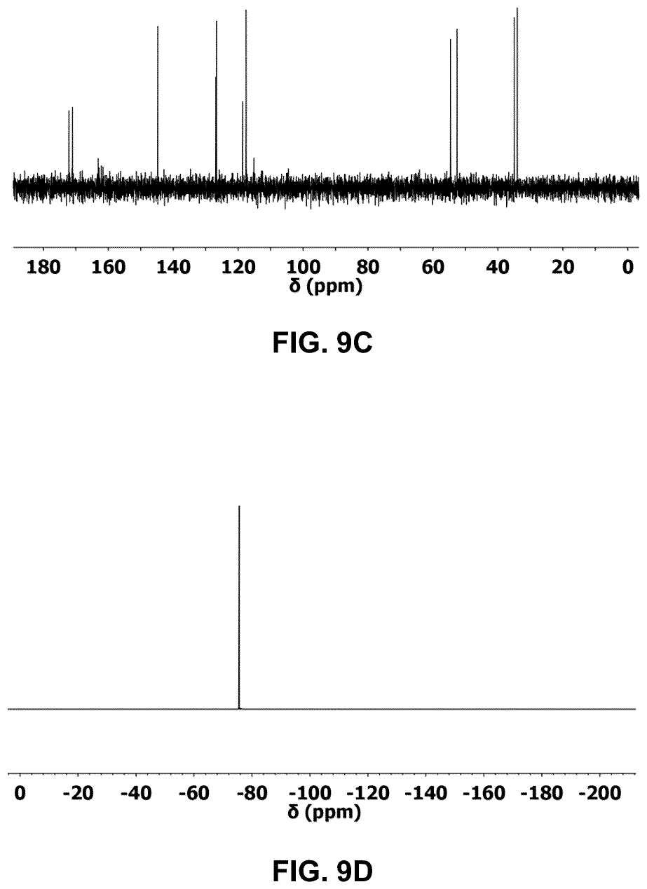

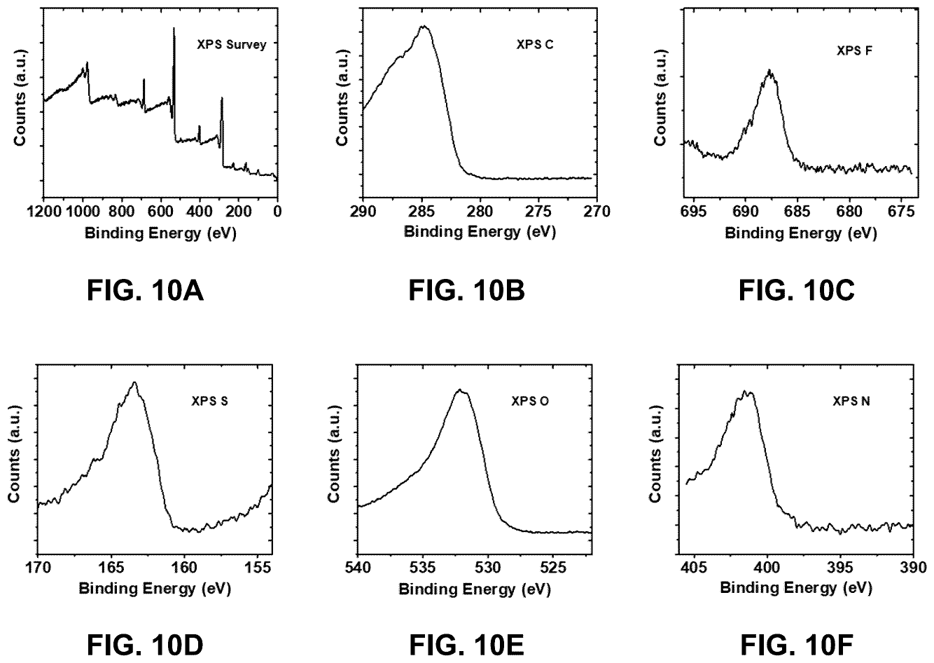

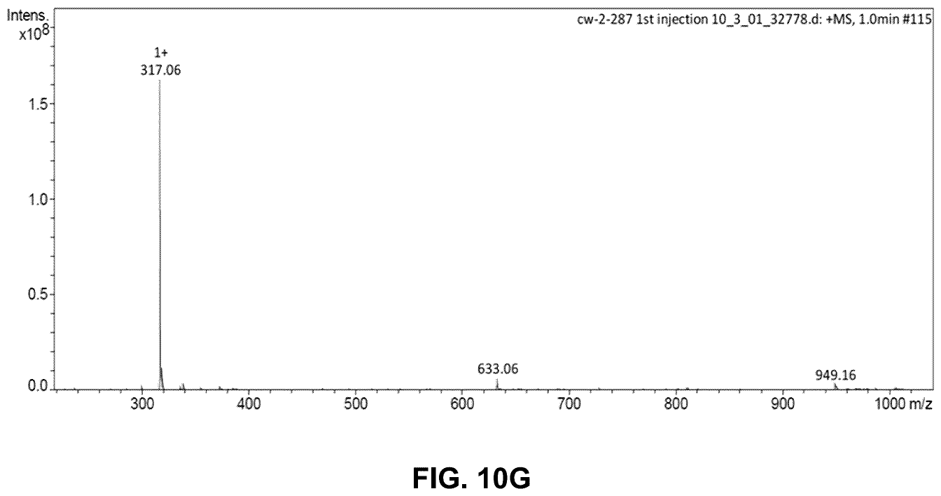

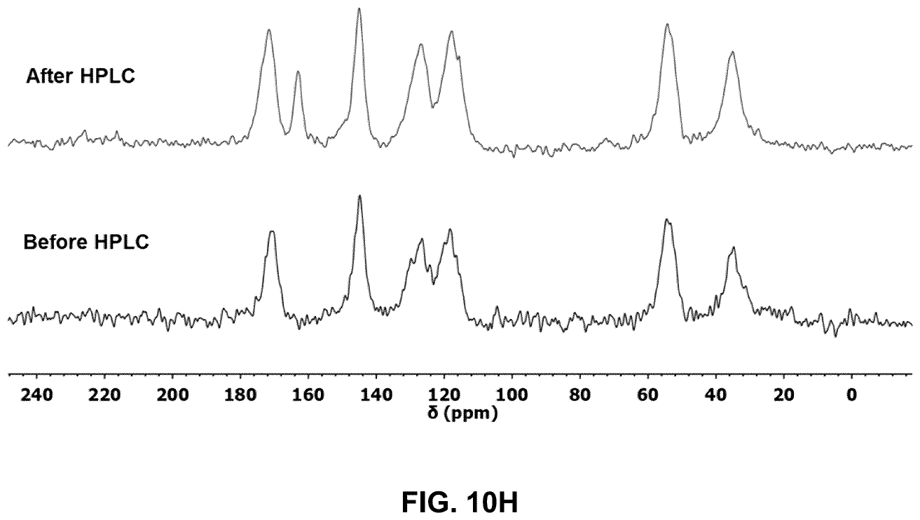

Our first objective was to identify a reliable synthetic method for artificial pheomelanin. We initially employed two routes (FIGS. 1A-1B). Method 1) KMnO₄ was used to oxidize a solution phase mixture of cysteine and _{L}-DOPA in water at pH 7.³² Method 2) Oxygen was used to oxidize cysteine and _{L}-DOPA in phosphate-buffered saline (PBS) in the presence of tyrosinase.³³ However, ¹³C ssNMR revealed no discernable benzothiazine-base motifs within the resulting structures (Error! Reference source not found. B), which are widely accepted as being present in natural pheomelanin.^{14, 25} We use ssNMR as the main characterization method because it can minimize disruptive sample preparation compared to other typical methods like chemical degradation and high-performance liquid chromatography (HPLC) separation.^{41, 42} To verify if the possible disulfide species hinder the pheomelanin formation, KMnO₄ was used to oxidize a solution phase mixture of cysteine and L-DOPA in water at pH 7, following the in situ reduction of cystine (FIGS. 8A-8B), yet this attempt resulted in a similar ssNMR spectra as method 1 and 2. The structural discrepancy of the well-established methods encouraged us to try a third method (Method 3), in which we employed 5-cysteinyl-DOPA (5-CD) as the starting monomeric material (FIG. 1A 3, and see FIGS. 9A-9D and 10A-10H for the synthesis and characterization of 5-CD).²⁸ Here we utilized a chemoenzymatic methodology with HRP as the enzyme and hydrogen peroxide as the oxidant. Zinc sulphate was added to the reaction to aid in retaining carboxylic acid groups in the benzothiazine intermediates.^{28, 31} As shown in Error! Reference source not found. B, only Method 3 yields a pheomelanin structure with reasonable ssNMR features. Previously, pheomelanin materials synthesized using these three methods have not been thoroughly compared. Our ssNMR data show that only the 5-CD method gives a close pheomelanin mimic, while using L-DOPA and cysteine does not.

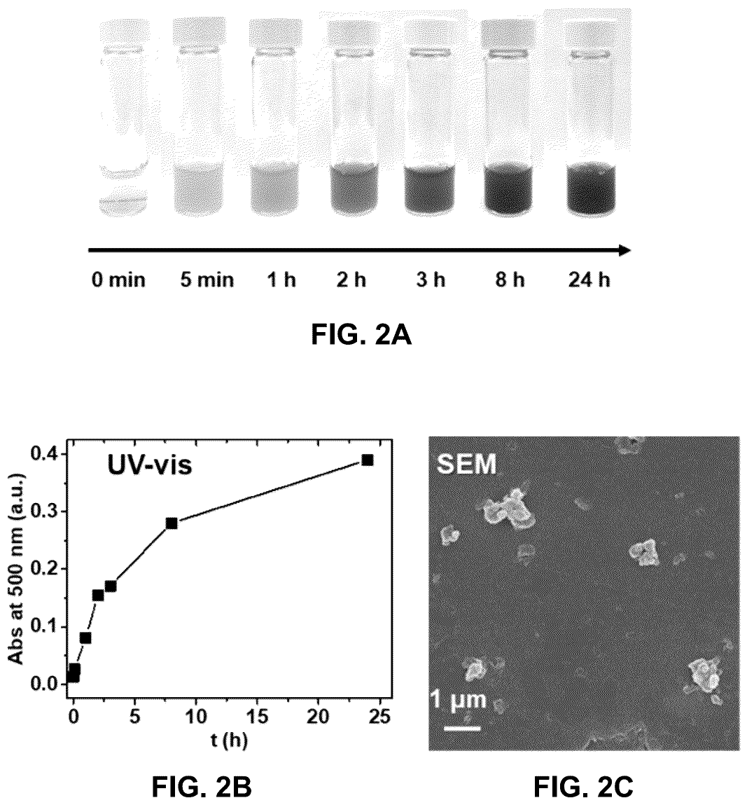

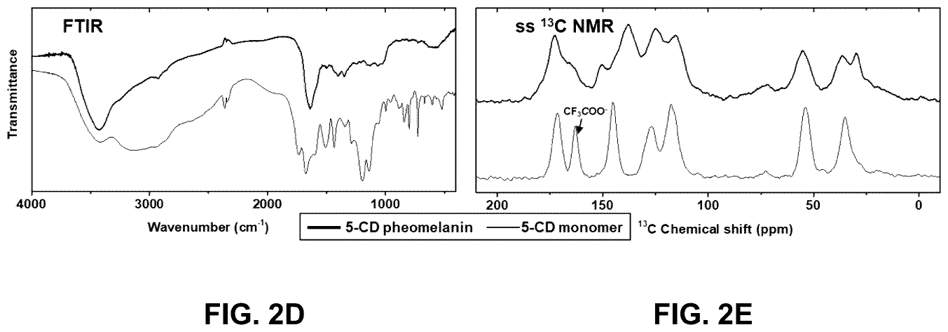

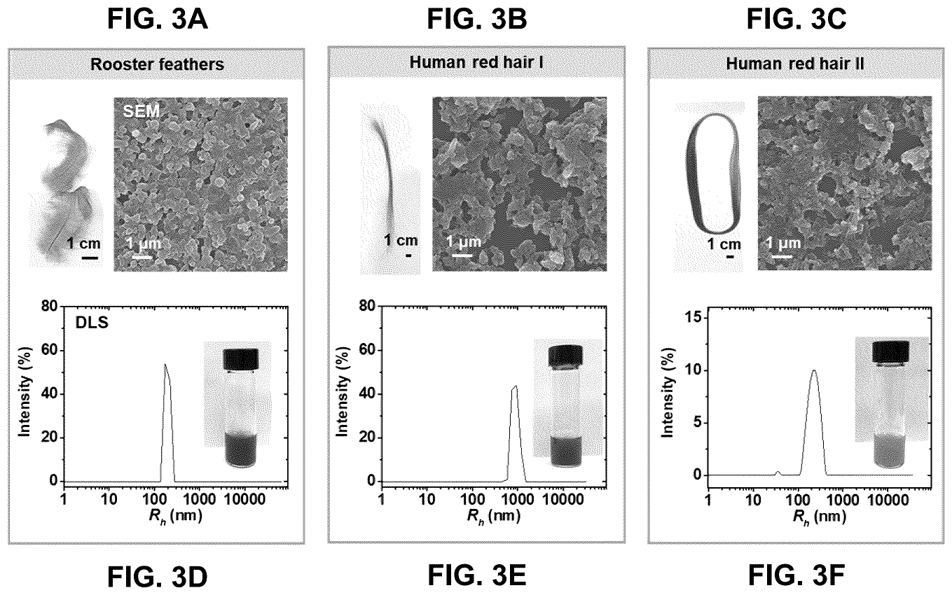

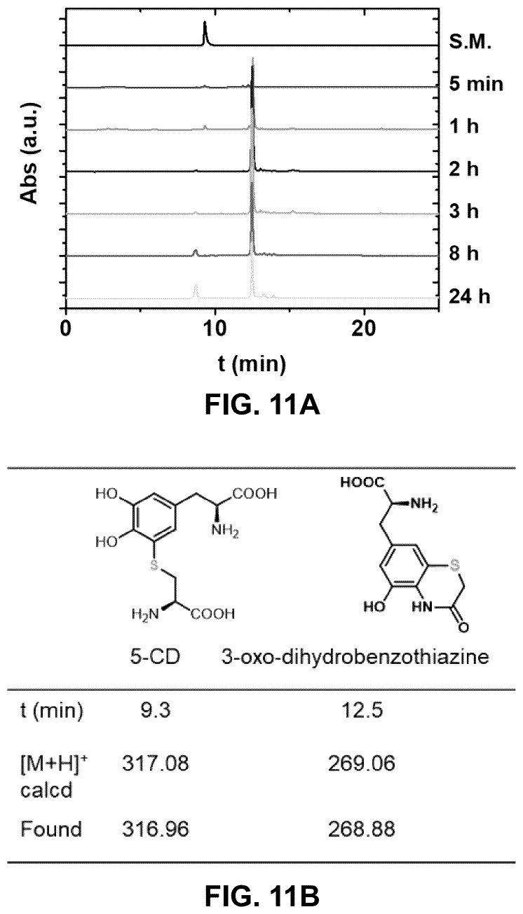

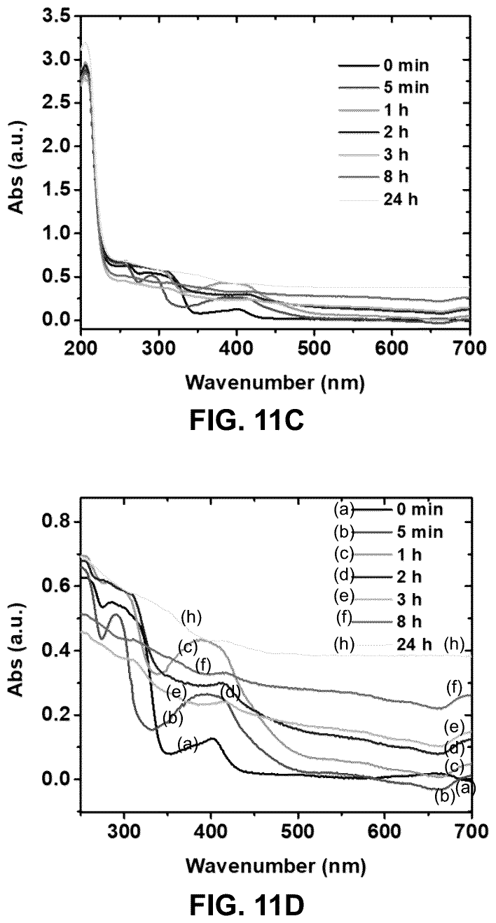

5-CD polymerization reaction proceeds from an initially clear, light-yellow solution becoming bright yellow upon mixing the H₂O₂ and HRP with 5-CD (FIG. 2A). The solution changed from yellow to orange, to red, and eventually to a dark reddish brown over 24 h. The color change is distinct from the conventional PDA or poly(L-DOPA)-based eumelanin synthesis which quickly results in a yellow, to dark brown and finally to a black solution.⁴³ As monitored by UV-vis spectroscopy (FIGS. 11A-11D), a peak around 390 nm developed during the first 5 min to 1 h period. This is followed by a more broadband absorption appearing after 2 h corresponding to an extension of the conjugated structure and more hierarchical polymers. This absorption spectroscopy change is consistent with previous observations by Napolitano.²⁸ Absorbance at 500 nm, which can be used to quantify the total amount of melanin,¹⁸ increased over the time course of the polymerization reaction (FIG. 2B). HPLC and electrospray ionization mass spectrometry (ESI-MS) data obtained periodically from aliquots of the reaction, revealed an expected 5-CD depletion (t_{R}=9.3 min, [M+H]⁺ m/z=316.96) and its conversion to the intermediate 3-oxo-3,4-dihydrobenzothiazine (t_{R}=12.5 min, [M+H]⁺ m/z=268.88) (FIGS. 11A-11D).

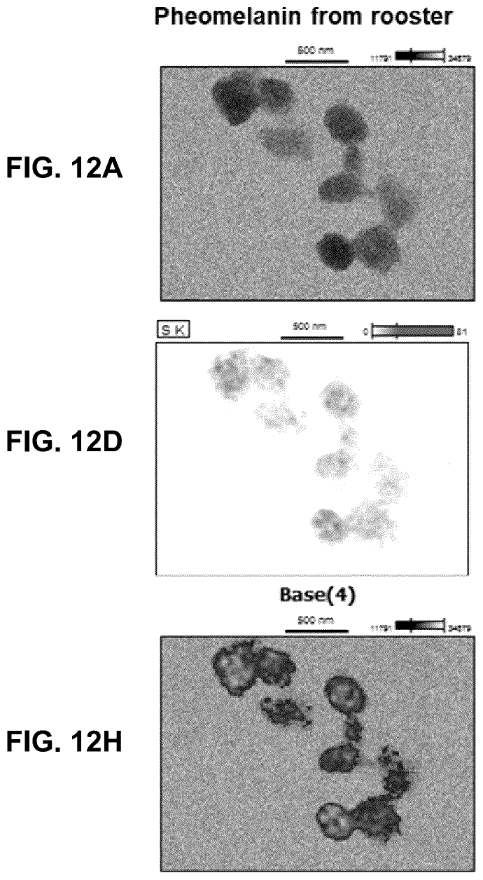

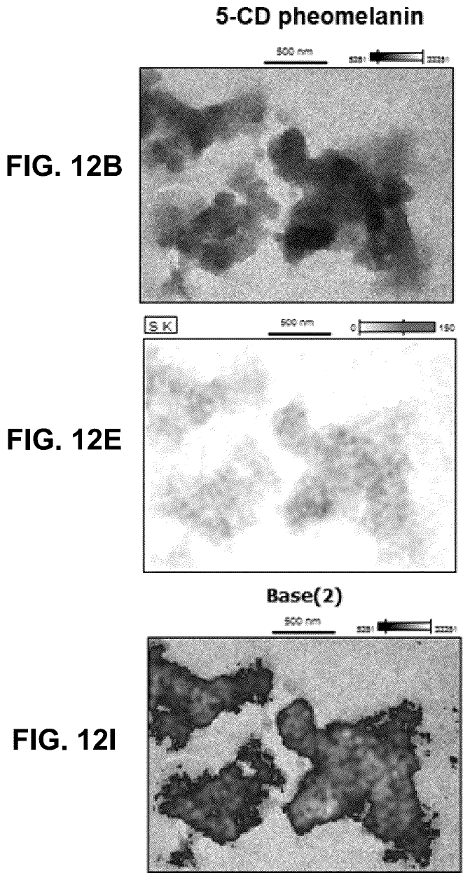

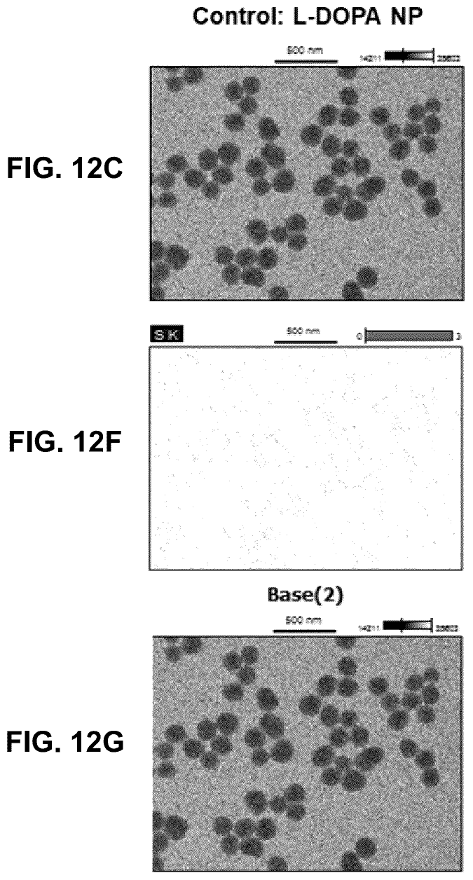

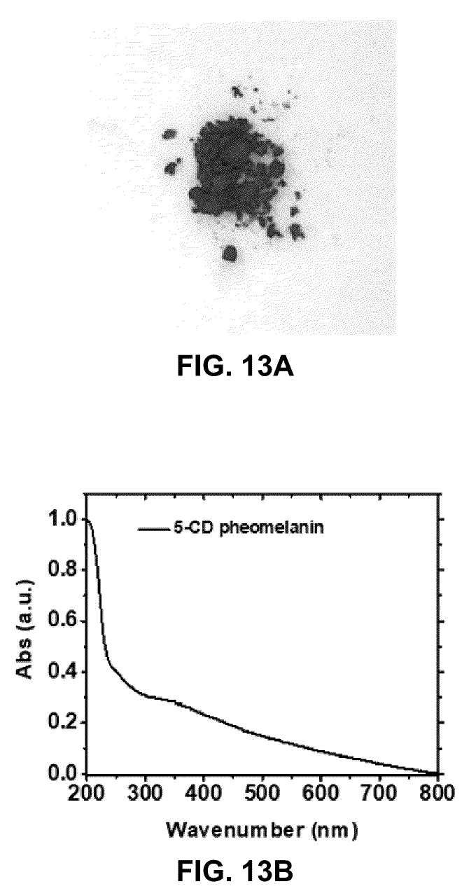

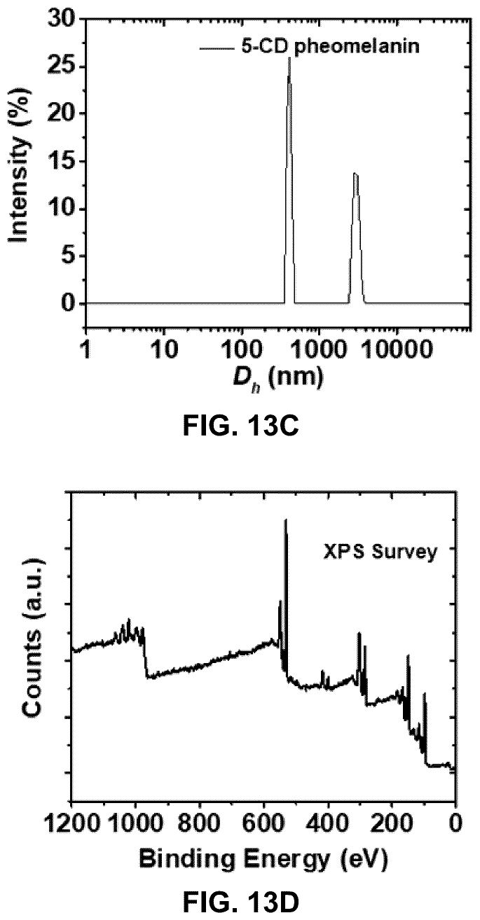

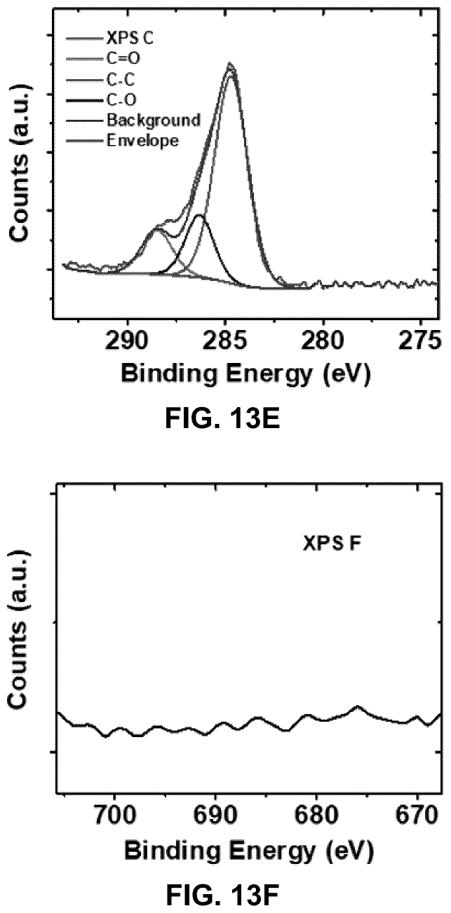

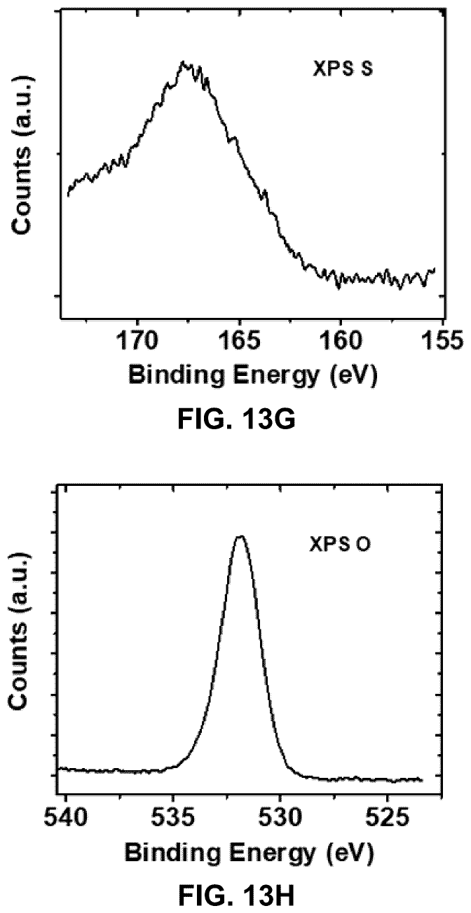

The reaction yielded irregular colloidal aggregates as characterized by scanning electron microscopy (SEM) (FIG. 2C) and scanning transmission electron microscopy (STEM) (FIGS. 12A-12I). DLS showed multiple peaks with a polydispersity index of ˜0.57 (FIGS. 13A-13I). The pheomelanin particles exhibited a negative ζ-potential of −27.3±1.8 mV due to the anionic phenol and carboxylic acid groups. X-ray Photoelectron Spectroscopy (XPS) provided non-destructive information for pheomelanin (FIGS. 13D-13I). Clear C, N, O and S signals could be identified, whereas the fluorine from the CF₃COO— counter ion on the 5-CD monomer disappeared after polymerization. Energy-dispersive X-ray spectroscopy (EDS) mapping via STEM imaging verified the colocalization of sulfur and the particulates (FIGS. 12A-12I). The resulting polymer shows broad absorption bands in the Fourier Transform Infrared (FTIR) spectrum (D), which contrasts with the peak shape of the 5-CD monomer. Typical absorption bands were observed in the 5-CD pheomelanin sample at 3700-2400 cm⁻¹ (stretching vibration of —OH, —COOH, —NH), 2920 cm⁻¹, 2850 cm⁻¹ (stretching vibration of aliphatic —CH, —CH₂), 1640 cm⁻¹ (bending vibrations of aromatic ring C═C, stretching vibration of —NH₂), 1400 cm⁻¹, 1345 cm⁻¹ (0-H bending of —COOH and phenol), and 1069 cm⁻¹, 1040 cm⁻¹ (aliphatic C—H deformation).

We subsequently used ¹³C ssNMR to compare the chemical structure of the synthetic pheomelanin with the 5-CD monomer (FIG. 2E). The peaks at 172.7 ppm and 165.7 ppm were ascribed to the —COOH groups from 5-CD moiety (¹³C ssNMR of 5-CD monomer is displayed for comparison), while the decrease in the peak at 150.3 ppm corresponds to a change from the catechol structure of the monomer to the o-aminophenol structure of the benzothiazine subunit. The peak at 137.7 ppm was assigned to aromatic signals belonging to benzothiazine units as reported of polycysteinyldopamine by Ambrico.⁴⁴ We note here that the spectral features in the carbonyl and aromatic regions are highly similar to that of a previously described selenomelanin, a benzoselenazine-based analogue.²³ In addition, the aliphatic moieties (20-60 ppm) in the 5-CD pheomelanin spectrum have a smaller integration area relative to that of 5-CD monomer, consistent with the expected conversion of sp³ carbons. Taken together, our results support the idea that the 5-CD polymerization proceeds according to the RMP and forms benzothiazine-based pheomelanin. Therefore, Method 3 should be used for future studies regarding pheomelanin function and applications.

Comparison of Synthetic Pheomelanin with Natural Pheomelanin from Different Sources

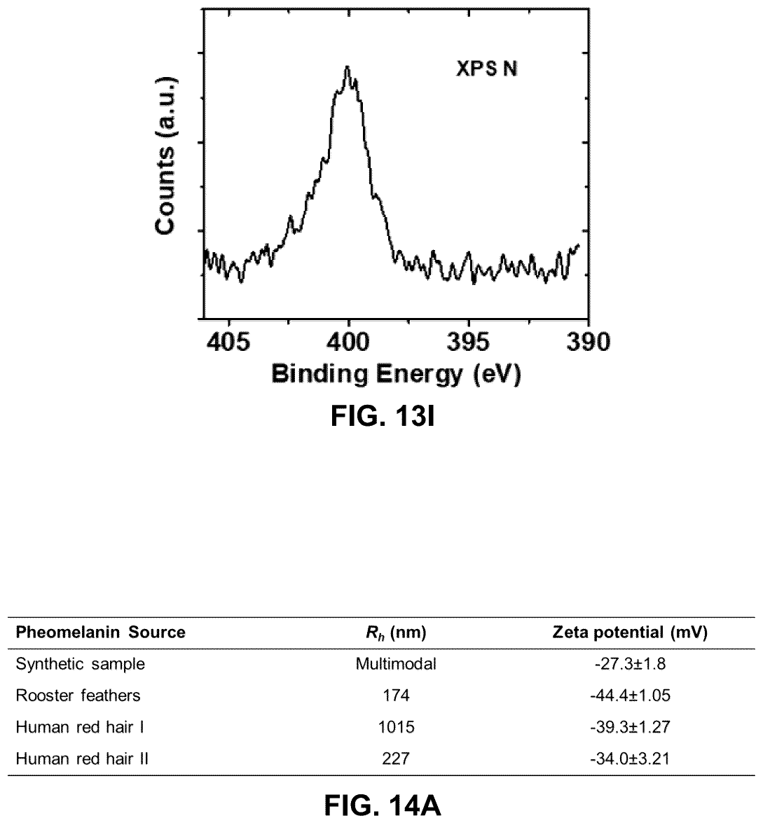

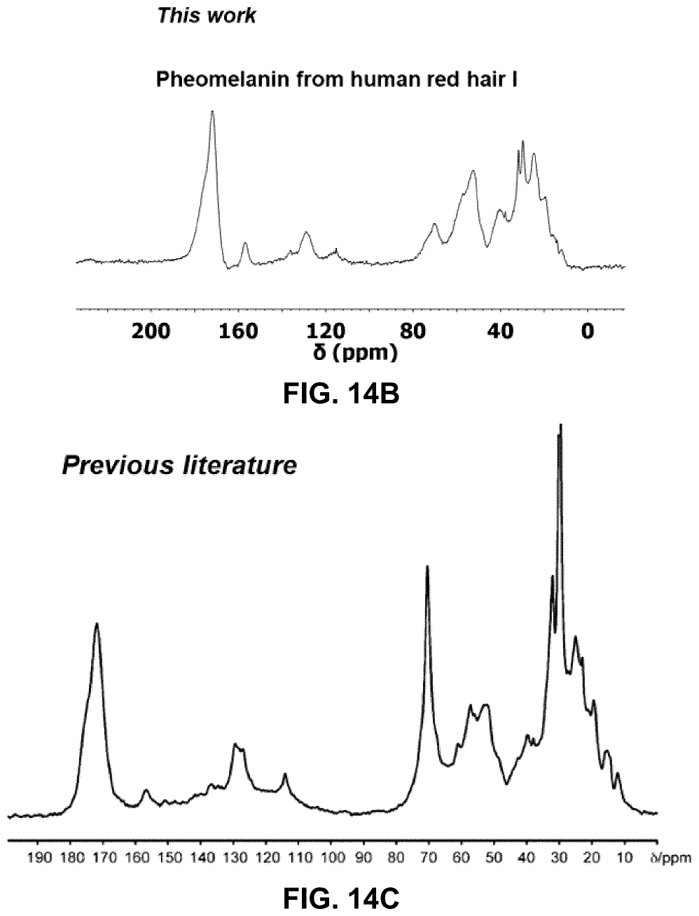

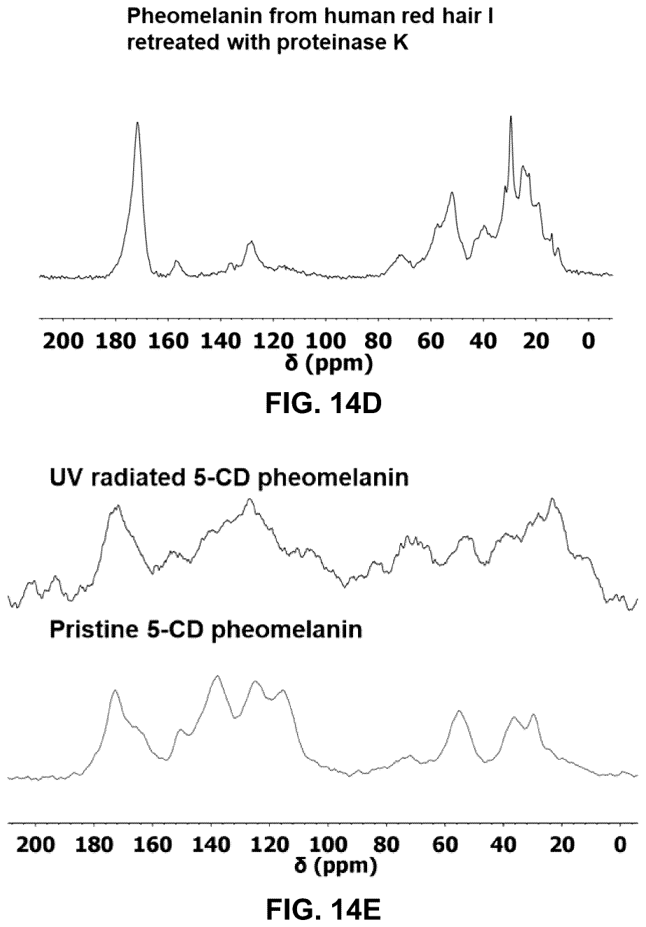

To perform a direct comparison of synthetic pheomelanin prepared from 5-CD with natural samples, pheomelanins were extracted from Rhode Island red rooster feathers, and human red hair from two separate individuals (Error! Reference source not found. 3A-3F). Enzymatic extraction was chosen over chemical extraction because the mild conditions are expected to preserve the melanin in its natural form far better than harsher conditions.⁴⁵ After a series of enzymatic treatment cycles and washing steps, the sample was characterized via various techniques including SEM, DLS, and ssNMR to provide information regarding the natural chemical structure. Particles from the natural pheomelanin samples exhibit a more irregular shape than extracted eumelanin nanomaterials, as previously reported.^{1, 21, 46} The size of the resulting particles varies with rooster feathers about 330 nm in diameter by SEM microscopy (Error! Reference source not found.A) and particles from human hair measuring in the micrometer length scale (Error! Reference source not found.C), which also agree with the DLS results (Error! Reference source not found.D-3F). Interestingly, we observed a size variation of the pheomelanin from two human donors likely due to the donor difference. ζ-potential of the natural pheomelanin samples ranges from −34 mV to −44 mV (FIG. 14A), similar to that of the synthetic sample.

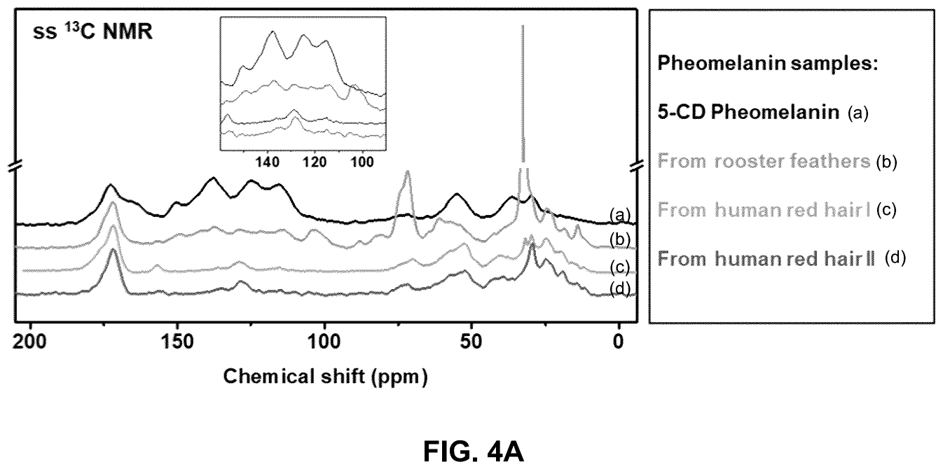

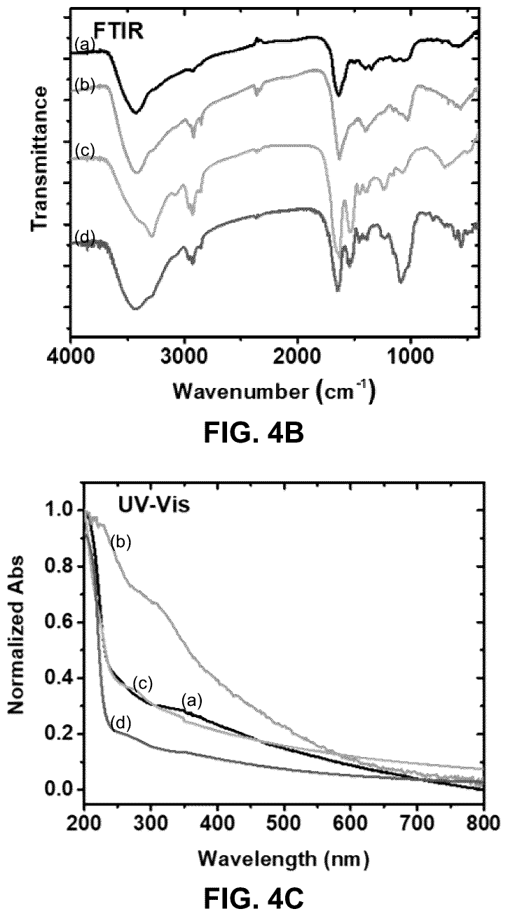

¹³C ssNMR of pheomelanin extracted from bird feathers showed a rough similarity in the aromatic region to that of the synthetic pheomelanin (FIG. 4A). ssNMR of hair pheomelanins is similar to that seen in previous literature examples (FIGS. 14B-14C).³⁰ There are some signals ascribed to proteinaceous components (172 ppm and the region 50 ppm-10 ppm). However, extra proteinase-K treatment does not significantly decrease the lipid signal intensities (FIG. 14D), which indicates that peptides/proteins may be physically trapped within the pheomelanin, rendering them less accessible to enzyme extraction.⁴⁷ In turn, three higher intensity peaks in the feather sample at ˜172 ppm, 72 ppm and 33 ppm were assigned to lipids. Similar assignments to lipids were reported by Stark in a fungal melanin sample.⁴⁷ Lipid peaks were also observed in the human hair samples, which is further supported by FTIR peaks at ˜2955 cm⁻¹ (aliphatic C—H stretch vibration, Error! Reference source not found.B). UV-vis spectra each showed broadband absorption (Error! Reference source not found.C), which is typical for melanin and melanin-like materials.²⁹ The absorption spectra from 250 nm to 800 nm of 5-CD pheomelanin and pheomelanins from red hair showed similar features to the previous observation by Napolitano.²⁸

These studies confirm the morphology and the chemical form by comparison of 5-CD based synthetic and natural pheomelanins.

However, there are limitations to this approach, as we could not distinguish benzothiazine and benzothiazole based on the ¹³C ssNMR. It is known that UVA irradiation could convert benzothiazine moieties to benzothiazole units in natural pheomelanin based on HPLC analysis.^{31, 48} We performed UV (365 nm, 7.0 mW·cm⁻²) irradiation of our 5-CD pheomelanin for 30 h and characterized them by ssNMR (FIG. 14E). Significant changes were observed in the aromatic region, which presumably results from the benzothiazine to benzothiazole conversion. Opportunities exist where isotope labeling and multi-dimensional ssNMR may reveal more structural information.

Surface Properties and Color Comparison of Synthetic Pheomelanin with PDA Mimics of Eumelanin

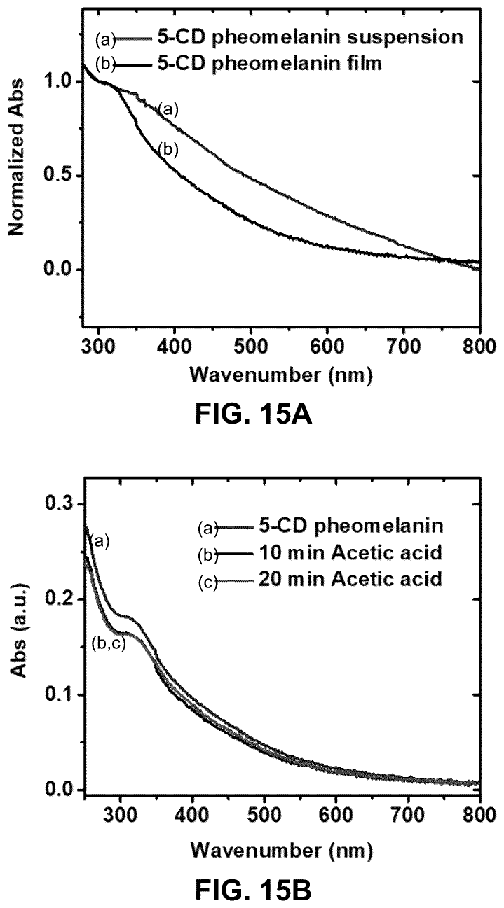

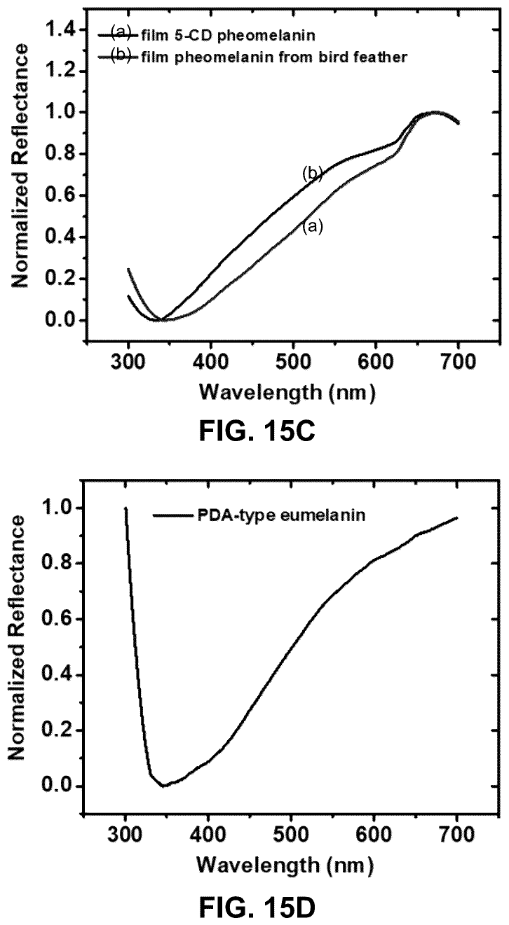

As the polydopamine-type eumelanin chemistry on surface has aroused broad interest,⁴⁹ we next set out to investigate whether synthetic pheomelanin led to different surface properties compared to PDA. Here we choose PDA as a synthetic eumelanin mimic because PDA is the most commonly-used and well-studied standard for eumelanin in literature.^{12, 49} In contrast to PDA-based film studies, pheomelanin films from close mimics of natural pheomelanin have not been studied.^{44, 50} Pheomelanin films were prepared by submerging a glass slide in the polymerization reaction solution (Error! Reference source not found.). PDA films were prepared similarly via the oxidative polymerization of dopamine under alkaline conditions in the presence of a glass slide.⁵¹ The water contact angle of pheomelanin was 20.7° (Error! Reference source not found.A), smaller than that of PDA (34.8°, Error! Reference source not found.B) and blank glass (25.5°, Error! Reference source not found.C), indicative of a higher hydrophilicity. Our data showed that a continuous pheomelanin film can also be prepared (Error! Reference source not found.D, see PDA control 4E and blank glass 4F). The synthetic film showed the typical broadband absorption seen in aqueous dispersion (FIGS. 15A-15D). Other substrates like polystyrene and gold substrates could also be coated (Error! Reference source not found.G). Additionally, the pheomelanin could also be deposited onto a 3D-printed poly-methacrylate object (Error! Reference source not found.H), showing the versatility of surface modification with these materials. Furthermore, synthetic pheomelanin appears reddish brown in color compared with the darker, black PDA at identical particle concentration by mass (1 mg/mL) (Error! Reference source not found.I). Reflectance spectra of the films made of synthetic and natural pheomelanin showed a peak around 670 nm, corresponding to the visual red color of pheomelanin (FIGS. 15A-15D). Considering the long-lasting enthusiasm of polydopamine chemistry on surfaces for numerous applications,^{37, 49, 52} these pheomelanin films should be of significant interest as a surface modification and photochemical responsive material.⁴⁴

Electronic Structure Comparison

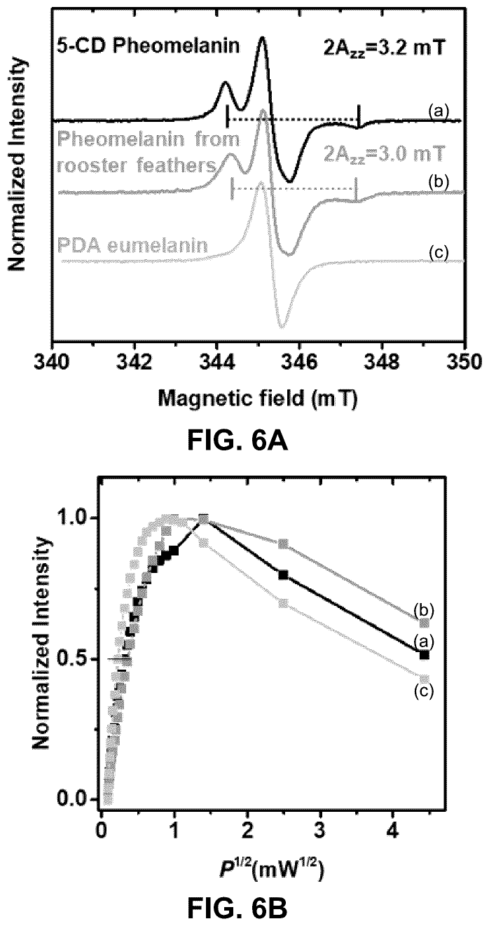

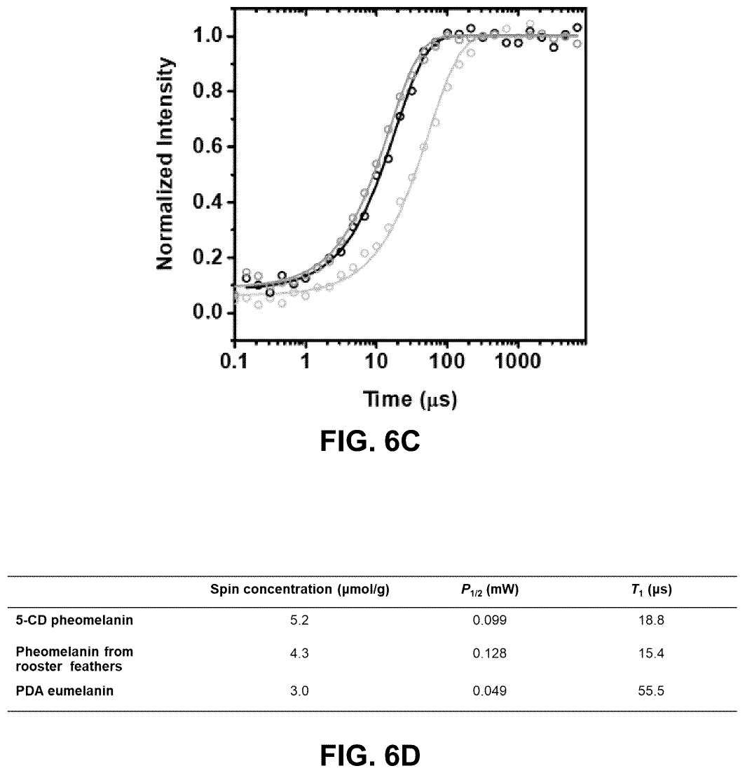

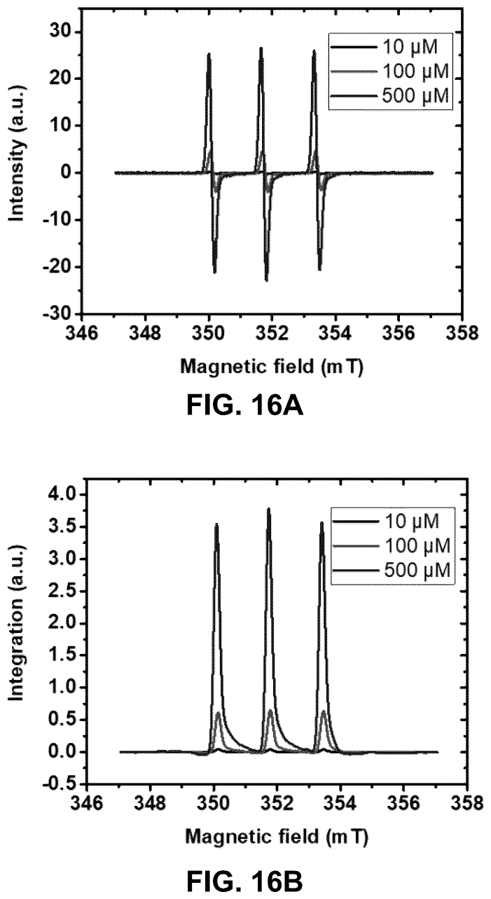

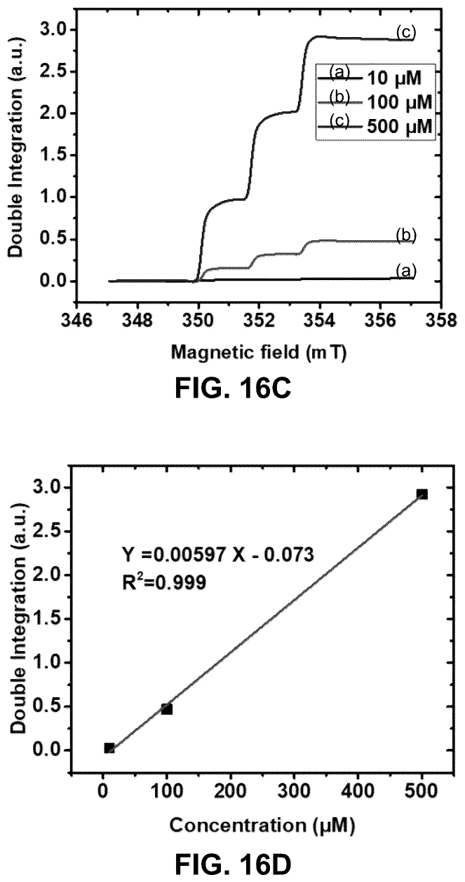

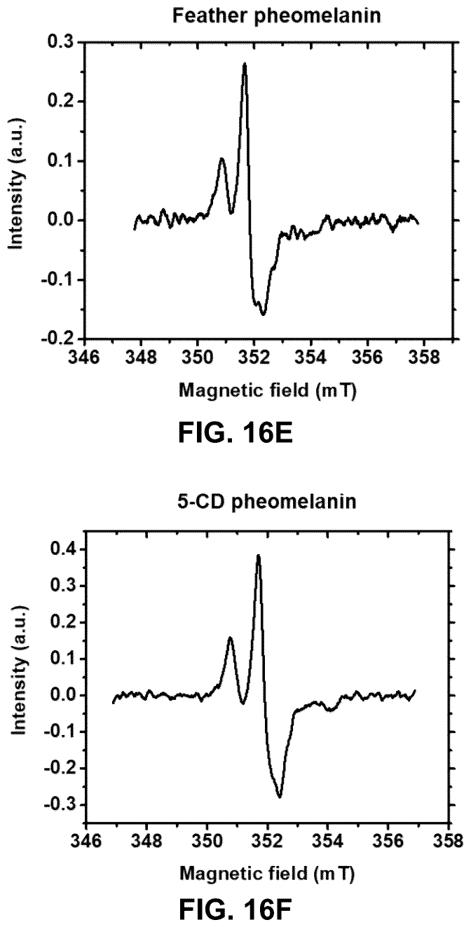

Electron paramagnetic resonance (EPR) reveals that stable free radicals exist in pheomelanin (FIGS. 6A-6D). The anisotropic nitrogen hyperfine splitting (Error! Reference source not found.A) is similar to that of natural pheomelanin extracted from rooster feathers, indicating a similar chemical structure. Despite the inhomogeneous broadening of the spectra, one of the principal values of the nitrogen hyperfine tensor, A_{zz},⁵³ can be read out directly from the peak-to-peak linewidth (Error! Reference source not found.A). The A_{zz} values extracted from the EPR spectra of 5-CD pheomelanin and feather pheomelanin are virtually the same, and are significantly lower than nitroxide radicals,⁵⁴ suggesting the existence of a semiquinoneimine radical at an o-aminophenol site, a signature for natural pheomelanin.⁵⁵ By contrast, PDA eumelaninspectra of the 5-CD pheomelanin and feather pheomelanin as aqueous dispersions (FIGS. 16A-16F) are similar to those of the powder form (Error! Reference source not found.A). In addition, quantitative EPR in dispersion elucidated that the spin concentration of the pheomelanin is ˜1.7 times higher than that of PDA-type eumelanin (FIGS. 16A-16F), consistent with previous studies.²²

To further study the difference between pheomelanin and eumelanin, a continuous-wave EPR power saturation curve was measured (Error! Reference source not found.B). In these plots, the signal amplitude of an EPR spectrum was plotted against the square root of the incident microwave power P. The signal amplitude increased linearly with the square root of P until it began to saturate, and then decreased in intensity. The 5-CD pheomelanin (Error! Reference source not found.B) behaved similarly to the pheomelanin from rooster feathers, whereas the slower saturation of PDA melanin (Error! Reference source not found.B) with increasing power compared with pheomelanin suggests a longer spin-lattice relaxation time caused by the different chemical structures. For PDA eumelanin, the P_{1/2}, which is the power at which the signal amplitude is half-saturated, is much smaller than the pheomelanin samples (Error! Reference source not found.D). To further quantify the spin-lattice relaxation time T₁, pulse EPR measurements were utilized. Pulse experiments can measure relaxation time more directly than the continuous-wave saturation experiment. The T₁ is 18.8 μs for synthetic pheomelanin (Error! Reference source not found.C), which agrees reasonably well with the feather pheomelanin (15.4 μs) and is much shorter than PDA eumelanin (55.5 μs, Error! Reference source not found.D).

Cation-π Interactions Elucidated by Controlled Disassembly

Although the RMP provides a molecular pathway for the synthesis of pheomelanin, the concomitant non-covalent pathway for pheomelanin remains elusive. Nature is known to use cation-π interactions to bind important small molecules, like acetylcholine.^{40, 56} In biomacromolecules like proteins, cation-π interactions make significant energetic contributions to protein stability.⁵⁷ In the Protein Data Bank, 1 out of 77 amino acid residues has cation-π interactions.⁴⁰ Melanin is a biomacromolecule that has conjugated r systems and cationic groups, making cation-π interactions readily accessible. For instance, it was reported that cation-π interactions were the primary mechanism for progressive assembly in PDA mimics of eumelanin.³⁹ Based on the chemical structure of pheomelanin, we hypothesize that cation-π interactions could exist in pheomelanin, along with the more commonly accepted hydrogen bonding and π-π interactions. Compared with the well-studied PDA, the supramolecular interactions of the pheomelanin are more difficult to decipher due to the lack of a well-established model system. Here, we utilized our synthetic pheomelanin films as a model system to study the supramolecular interactions in pheomelanin.

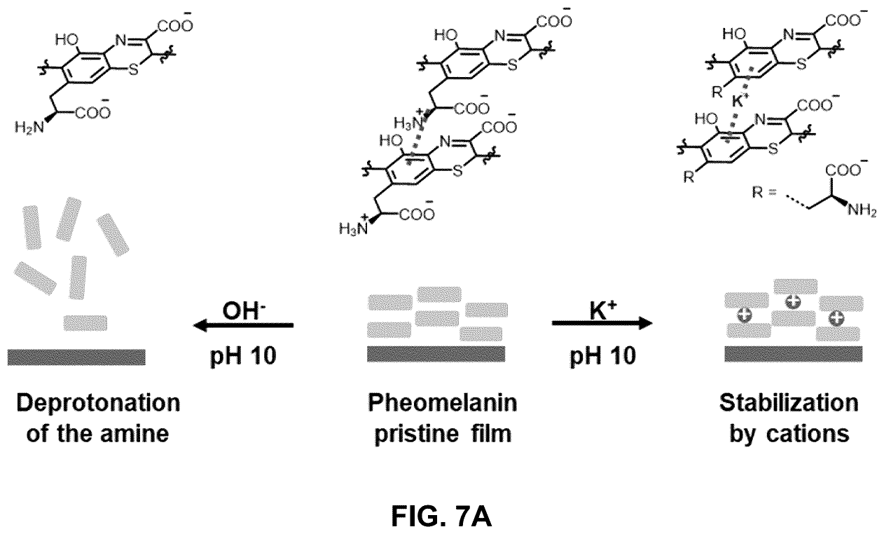

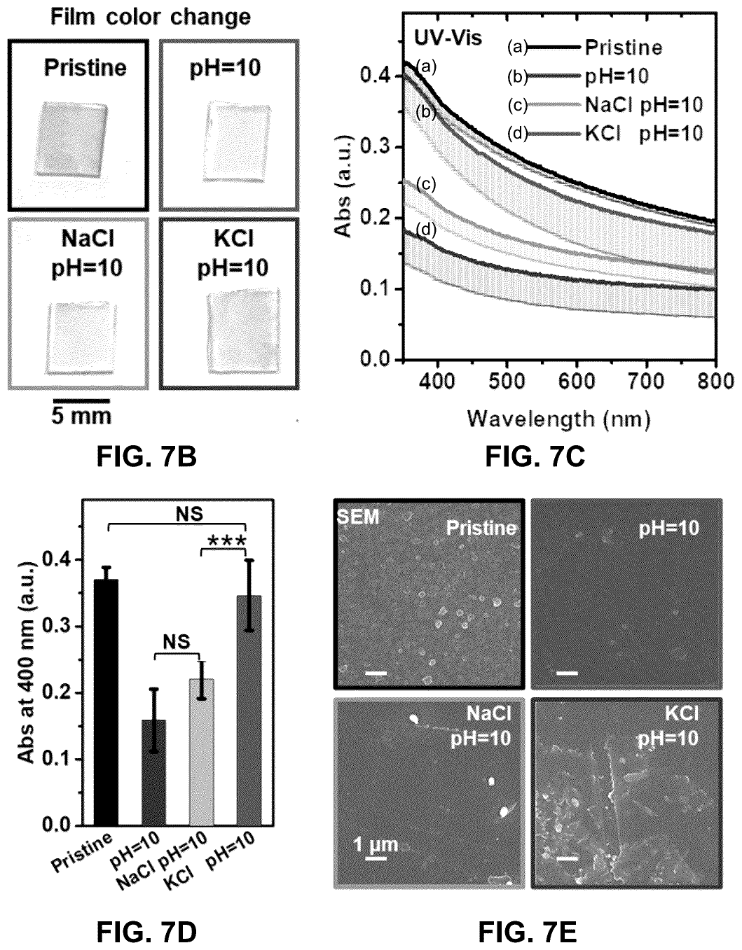

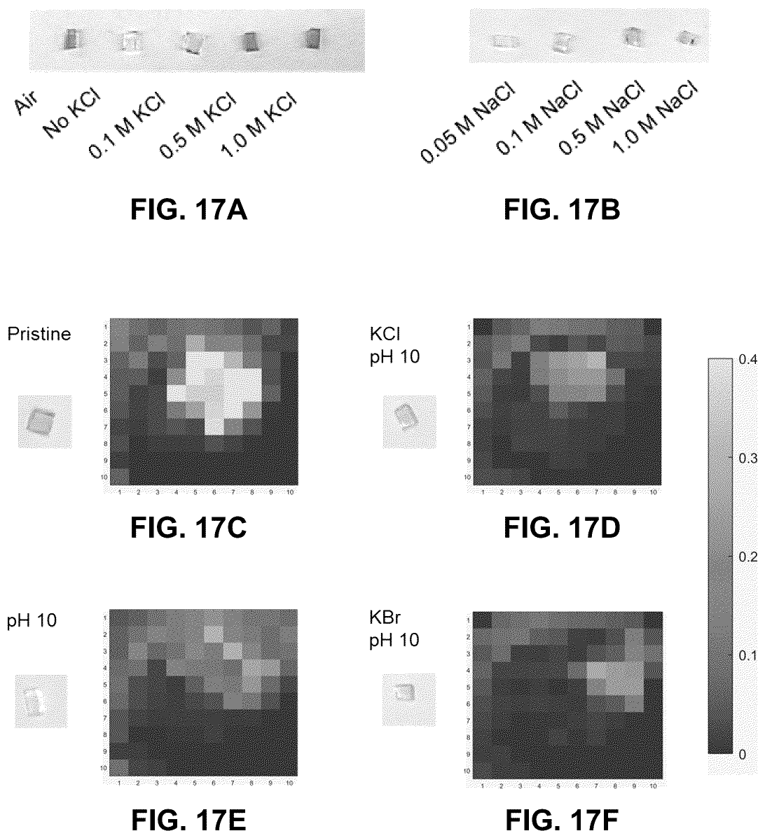

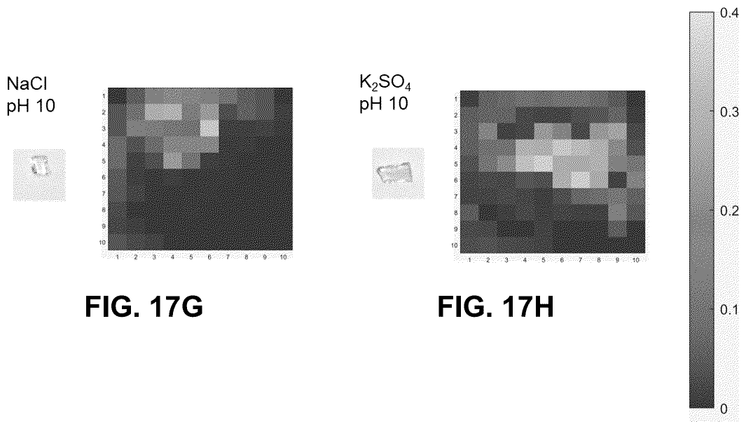

We speculated that deprotonation of the cationic ammonium might result in disassembly of the entire pheomelanin thin film if cation-π bonding is the dominant driving force for pheomelanin supramolecular formation (FIGS. 7A-7E). First, we found that KOH aqueous solution (pH 10) could quickly trigger the disassembly of the pheomelanin film (Error! Reference source not found.A-7B),⁵⁸ whereas the film is stable in deionized water, pH 7.4 buffer and 1% acetic acid solution (FIGS. 15A-15D), suggesting the high stability at neutral or acidic environment. Secondly, the solution salinity strongly impacts the disassembly process under identical pH conditions. The addition of KCl (0.5 M) provides a stronger cation-π interaction, and therefore can compensate for the deprotonation of cationic amine (Error! Reference source not found.B). Furthermore, lowering potassium concentrations leads to a smaller compensation effect (FIGS. 17A-17H). By contrast, NaCl (0.5 M) showed only a very small compensation effect for disassembly because the Na⁺-π interaction is weaker than the K⁺-π interaction.⁵⁹ UV-vis spectra were recorded of the resulting film (Error! Reference source not found.C), and the statistical test showed that 0.5 M KCl treated film has the same absorbance at 400 nm with the pristine one (P=0.41, Error! Reference source not found.D), whereas the 0.5 M NaCl and pH 10 solution led to much lower absorbance values (P=0.0002, 0.0005 with reference to 0.5 M KCl treated film). After the salt treatment, SEM micrographs (Error! Reference source not found.E) confirmed that the film was mostly preserved in KCl solution, slightly preserved in NaCl solution, and only blank glass substrate could be observed under SEM after treatment with a pH 10 KOH solution. Additionally, to decouple this cation compensation effect from the type of anion, we then screened different counterions including Br⁻ and SO₄²⁻ (FIGS. 17A-17H). Similarly colored films were observed for alternate anions as compared to treatment with the KCl solution, and absorption mapping at 400 nm of the film supported a similar trend (FIGS. 17A-17H), indicating that the cationic species is the dominating factor in the pheomelanin disassembly process. The pKₐ of phenol hydroxyl group in tyrosine is 10.10. The pKₐ of —COOH is 1.91 and 2.18 for cystine and tyrosine. Therefore, the phenol remains protonated and —COOH remains deprotonated at the experimental conditions: pH 7 and pH 10.0. The disassembly of pheomelanin triggered by pH change from 7 to 10.0 indicates that the deprotonation of ammonium is the dominant factor for this change. The results collectively suggest the cation-π interaction contributes in a dominant fashion to the assembly of the pheomelanin pigment. Understanding the supramolecular pathway of pheomelaningenesis could lead to novel routes for manipulating artificial pheomelanin synthetically. This high-fidelity pheomelanin model also could enable the elucidation of other enigmatic properties and biological functions of pheomelanin.

### Conclusions

Biosynthetic and synthetic attempts at pheomelanin have been investigated multiple times over several decades by different groups,^{19, 27, 28} with most efforts focused on reaction intermediate studies and degradation product identification.^{28, 60} Yet thorough characterization of the resulting polymeric materials with reference to the natural product has been lacking. Here, we have verified a reliable method for preparing synthetic pheomelanin by comparison with enzymatically extracted natural pheomelanin samples from various sources utilizing non-destructive characterization methods, including ssNMR, FTIR, UV-vis and the powerful EPR approach, to compare with the 5-CD derived synthetic pheomelanin. We demonstrated that the chemoenzymatic oxidation of 5-CD could yield benzothiazine-based pheomelanin, but using cysteine and _{L}-DOPA failed, possibly because of the slow redox step to give cysteinyldopaquinone.²⁵ Although the 5-CD synthetic method is only one step further in the RMP, it altered the polymerization pathway significantly,²⁷ likely due to the suppression of the addition of cysteine to the quickly growing polymer chains.¹⁹

In nature, pheomelanin is found as a mixture of melanins according to the previously proposed “casing” model (pheomelanin cores encased by eumelanin surfaces).^{14, 26} This makes it a formidable challenge to study the properties of the pheomelanin polymer from natural sources. Synthetic methods, such as the use of 5-CD monomers, provide a route to circumvent the casing model since no eumelanin monomer is involved. As eumelanin typically has a well-defined shape, the absence of the casing eumelanin may account for the irregular morphology of 5-CD synthetic pheomelanin. To be clear, we note that despite structural similarity at the molecular level, morphologically the chemoenzymatic approach to synthetic pheomelanin yields irregular structures as opposed to the largely oval shapes of natural, isolated pheomelanin. Presumably, this is because biological processes guide the in vivo pheomelanogenesis utilizing confinement (liposomal, or casing induced) and genetically programmed progression of the melanosome. To truly mimic the shape and chemistry of the pheomelanosomes, synthetic templation or self-assembled nanoarchitecture approaches should be developed. Insight derived from our study highlighting the importance of cation-π interactions may give direction to such an approach.

In summary, 5-cysteinyl-DOPA oxidation under enzymatic conditions yields a close structural mimic to natural pheomelanin. Notably, synthetic pheomelanin was found to be more hydrophilic, exhibits a lower T₁ relaxation time than polydopamine-type eumelanin and is a reddish-brown color at the same mass concentration. Alongside the extensive structural characterization of pheomelanin, with this synthetic tool in hand, we found that cation-π interactions are an important driving force for the formation of pheomelanin. This insight may provide a deeper understanding of the melanogenesis of one of the most important melanin subfamilies in nature. Further research will focus on controlling monomer composition, introducing other functionalities, and controlling nanoscale/microscale morphology. Moreover, the pursuit of better synthetic mimics for melanin classes, including for eumelanin, where it is known that PDA is a common but incomplete analogue, promises to shed much needed light on function and molecular structure of melanins more generally.¹²

### REFERENCES CORRESPONDING TO EXAMPLE 1A

1. L. D'Alba and M. D. Shawkey, Physiol. Rev., 2019, 99, 1-19.
2. M. Xiao, Z. Hu, Z. Wang, Y. Li, A. D. Tormo, N. Le Thomas, B. Wang, N. C. Gianneschi, M. D. Shawkey and A. Dhinojwala, Sci. Adv., 2017, 3, e1701151.
3. A. Lampel, S. A. McPhee, H.-A. Park, G. G. Scott, S. Humagain, D. R. Hekstra, B. Yoo, P. W. J. M. Frederix, T.-D. Li, R. R. Abzalimov, S. G. Greenbaum, T. Tuttle, C. Hu, C. J. Bettinger and R. V. Ulijn, Science, 2017, 356, 1064-1068.
4. A. B. Mostert, S. B. Rienecker, C. Noble, G. R. Hanson and P. Meredith, Sci. Adv., 2018, 4, eaaq1293.
5. Y. Huang, Y. Li, Z. Hu, X. Yue, M. T. Proetto, Y. Jones and N. C. Gianneschi, ACS Cent. Sci., 2017, 3, 564-569.
6. X. Zhou, N. C. McCallum, Z. Hu, W. Cao, K. Gnanasekaran, Y. Feng, J. F. Stoddart, Z. Wang and N. C. Gianneschi, ACS Nano, 2019, 13, 10980-10990.
7. H. Lee, B. P. Lee and P. B. Messersmith, Nature, 2007, 448, 338-341.
8. Z. Wang, T. Tschirhart, Z. Schultzhaus, E. E. Kelly, A. Chen, E. Oh, O. Nag, E. R. Glaser, E. Kim, P. F. Lloyd, P. T. Charles, W. Li, D. Leary, J. Compton, D. A. Phillips, A. Dhinojwala, G. F. Payne and G. J. Vora, Appl. Environ. Microbiol., 2019, 86, e02749-02719.
9. Q. Li, K.-Q. Gao, Q. Meng, J. A. Clarke, M. D. Shawkey, L. D'Alba, R. Pei, M. Ellison, M. A. Norell and J. Vinther, Science, 2012, 335, 1215-1219.
10. J. Lindgren, P. Sjovall, R. M. Carney, P. Uvdal, J. A. Gren, G. Dyke, B. P. Schultz, M. D. Shawkey, K. R. Barnes and M. J. Polcyn, Nature, 2014, 506, 484-488.
11. H. Liu, Y. Yang, Y. Liu, J. Pan, J. Wang, F. Man, W. Zhang and G. Liu, Adv. Sci., 2020, 1903129.
12. W. Cao, X. Zhou, N. C. McCallum, Z. Hu, Q. Z. Ni, U. Kapoor, C. M. Heil, K. S. Cay, T. Zand, A. J. Mantanona, A. Jayaraman, A. Dhinojwala, D. D. Deheyn, M. D. Shawkey, M. D. Burkart, J. D. Rinehart and N. C. Gianneschi, J. Am. Chem. Soc., 2021, 143, 2622-2637.
13. X. Zhou, X. Gong, W. Cao, C. J. Forman, J. Oktawiec, L. D'Alba, H. Sun, M. P. Thompson, Z. Hu, U. Kapoor, N. C. McCallum, C. D. Malliakas, O. K. Farha, A. Jayaraman, M. D. Shawkey and N. C. Gianneschi, Angew. Chem. Int. Ed., 2021, 60, 17464-17471.
14. J. D. Simon and D. N. Peles, Acc. Chem. Res., 2010, 43, 1452-1460.
15. S. Ito and K. Fujita, Anal. Biochem., 1985, 144, 527-536.
16. Q. Ni, B. Sierra, J. J. LaClair and M. Burkart, Chem. Sci., 2020, 11, 7836-7841.
17. J. Wang and L. Blancafort, Angew. Chem. Int. Ed., 2021, 60, 18800-18809.
18. I. Galven, A. Jorge, F. Solano and K. Wakamatsu, Spectrochim. Acta. A Mol. Biomol. Spectrosc., 2013, 110, 55-59.
19. R. B. Deibel and M. R. Chedekel, J. Am. Chem. Soc., 1982, 104, 7306-7309.
20. F. Solano, Polym. Int., 2016, 65, 1276-1287.
21. T. H. Nasti and L. Timares, Photochem. Photobiol., 2015, 91, 188-200.
22. A. D. Schweitzer, R. C. Howell, Z. Jiang, R. A. Bryan, G. Gerfen, C. C. Chen, D. Mah, S. Cahill, A. Casadevall and E. Dadachova, PloS One, 2009, 4, e7229.
23. W. Cao, N. C. McCallum, Q. Z. Ni, W. Li, H. Boyce, H. Mao, X. Zhou, H. Sun, M. P. Thompson, C. Battistella, M. R. Wasielewski, A. Dhinojwala, M. D. Shawkey, M. D. Burkart, Z. Wang and N. C. Gianneschi, J. Am. Chem. Soc., 2020, 142, 12802-12810.
24. X. Chen, D. Feng, M. A. Schwarzschild and X. Gao, Ann. Clin. Transl. Neurol., 2017, 4, 212-216.
25. S. Ito, Anal. Biochem., 2003, 16, 230-236.
26. W. D. Bush, J. Garguilo, F. A. Zucca, A. Albertini, L. Zecca, G. S. Edwards, R. J. Nemanich and J. D. Simon, Proc. Natl. Acad. Sci. U.S.A., 2006, 103, 14785-14789.
27. R. M. B. Deibel and M. R. Chedekel, J. Am. Chem. Soc., 1984, 106, 5884-5888.
28. A. Napolitano, M. De Lucia, L. Panzella and M. d'Ischia, Photochem. Photobiol., 2008, 84, 593-599.
29. M. d'Ischia, A. Napolitano, A. Pezzella, P. Meredith and M. Buehler, Angew. Chem. Int. Ed., 2020, 59, 11196-11205.
30. P. Thureau, F. Ziarelli, A. Thevand, R. W. Martin, P. J. Farmer, S. Viel and G. Mollica, Chem. Eur. J., 2012, 18, 10689-10700.
31. A. Napolitano, L. Panzella, G. Monfrecola and M. d'Ischia, Pigm Cell Melanoma R, 2014, 27, 721-733.
32. J. Pyo, K. Y. Ju and J. K. Lee, J Photochem. Photobiol. B, 2016, 160, 330-335.
33. S. Ito, Pigment Cell Res., 1989, 2, 53-56.
34. R. C. Sealy, J. S. Hyde, C. C. Felix, I. A. Menon and G. Prota, Science, 1982, 217, 545-547.
35. A. Büngeler, B. Hsmisch and O. I. Strube, Int. J. Mol. Sci., 2017, 18, 1901.
36. S. Hong, Y. S. Na, S. Choi, I. T. Song, W. Y. Kim and H. Lee, Adv. Funct. Mater., 2012, 22, 4711-4717.
37. M. d'Ischia, A. Napolitano, V. Ball, C. T. Chen and M. J. Buehler, Acc. Chem. Res., 2014, 47, 3541-3550.
38. L. Panzella, G. Gentile, G. D'Errico, N. F. Della Vecchia, M. E. Errico, A. Napolitano, C. Carfagna and M. d'Ischia, Angew. Chem. Int. Ed., 2013, 52, 12684-12687.
39. S. Hong, Y. Wang, S. Y. Park and H. Lee, Sci. Adv., 2018, 4, eaat7457.
40. D. A. Dougherty, Acc. Chem. Res., 2013, 46, 885-893.
41. S. Tian, J. Garcia-Rivera, B. Yan, A. Casadevall and R. E. Stark, Biochemistry, 2003, 42, 8105-8109.
42. W. Li, Z. Wang, M. Xiao, T. Miyoshi, X. Yang, Z. Hu, C. Liu, S. S. C. Chuang, M. D. Shawkey, N. C. Gianneschi and A. Dhinojwala, Biomacromolecules, 2019, 20, 4593-4601.
43. K.-Y. Ju, Y. Lee, S. Lee, S. B. Park and J.-K. Lee, Biomacromolecules, 2011, 12, 625-632.
44. M. Ambrico, N. F. Della Vecchia, P. F. Ambrico, A. Cardone, S. R. Cicco, T. Ligonzo, R. Avolio, A. Napolitano and M. d'Ischia, Adv. Funct. Mater., 2014, 24, 7161-7172.
45. Y. Liu, V. R. Kempf, J. Brian Nofsinger, E. E. Weinert, M. Rudnicki, K. Wakamatsu, S. Ito and J. D. Simon, Pigment Cell Res., 2003, 16, 355-365.
46. Y. Liu, L. Hong, K. Wakamatsu, S. Ito, B. Adhyaru, C. Y. Cheng, C. R. Bowers and J. D. Simon, Photochem. Photobiol., 2005, 81, 135-144.
47. S. Chatterjee, R. Prados-Rosales, S. Tan, V. C. Phan, C. Chrissian, B. Itin, H. Wang, A. Khajo, R. S. Magliozzo, A. Casadevall and R. E. Stark, J. Biol. Chem., 2018, 293, 20157-20168.
48. K. Wakamatsu, Y. Nakanishi, N. Miyazaki, L. Kolbe and S. Ito, Pigment Cell Melanoma Res, 2012, 25, 434-445.
49. J. H. Ryu, P. B. Messersmith and H. Lee, ACS Appl. Mater. Inter., 2018, 10, 7523-7540.
50. N. F. Della Vecchia, R. Marega, M. Ambrico, M. Iacomino, R. Micillo, A. Napolitano, D. Bonifazi and M. d'Ischia, J. Mater. Chem. C, 2015, 3, 6525-6531.
51. H. Lee, S. M. Dellatore, W. M. Miller and P. B. Messersmith, Science, 2007, 318, 426-430.
52. P. Yang, F. Zhu, Z. Zhang, Y. Cheng, Z. Wang and Y. Li, Chem. Soc. Rev., 2021, 50, 8319-8343.
53. E. Bordignon, eMagRes, 2017, 6, 235-254.
54. O. H. Griffith, D. W. Cornell and H. M. McConnell, J. Chem. Phys., 1965, 43, 2909-2910.
55. R. C. Sealy, J. S. Hyde, C. C. Felix, I. A. Menon, G. Prota, H. M. Swartz, S. Persad and H. F. Haberman, Proc. Natl. Acad. Sci. U.S.A, 1982, 79, 2885-2889.
56. W. Zhong, J. P. Gallivan, Y. Zhang, L. Li, H. A. Lester and D. A. Dougherty, Proc. Natl. Acad. Sci. U.S.A, 1998, 95, 12088-12093.
57. J. P. Gallivan and D. A. Dougherty, Proc. Natl. Acad. Sci. U.S.A., 1999, 96, 9459-9464.
58. R. D. Lillie, J. Histochem. Cytochem., 1955, 3, 453-454.
59. Q. Lu, D. X. Oh, Y. Lee, Y. Jho, D. S. Hwang and H. Zeng, Angew. Chem. Int. Ed., 2013, 52, 3944-3948.
60. A. Napolitano, S. Memoli and G. Prota, J. Org. Chem., 1999, 64, 3009-3011.

### Example 1B: Supporting Information for Biomimetic Pheomelanin to Unravel the Electronic, Molecular and Supramolecular Structure of the Natural Product

Materials and Methods

Materials

_{L}-3,4-dihydroxyphenylalanine (L-DOPA), tris(2-carboxyethyl) phosphine hydrochloride (TCEP-HCl), cysteine, cystine and Dulbecco's phosphate buffered saline (DPBS) were purchased from Fisher Scientific. Hydrogen peroxide, horseradish peroxidase (HRP), Triton™ X-100, Papain from papaya latex was purchased from Sigma-Aldrich. Proteinase-K was purchased from Gold Biotechnology. Human red hair was donated by a Caucasian male (red hair I) and female (red hair II) affiliated with Northwestern University. All reagents and materials were used as received unless otherwise stated.

Instrumentation

Solution Nuclear Magnetic Resonance (NMR) characterization. ¹H NMR spectra and ¹⁹F NMR spectra were recorded on a Bruker Avance III HD system equipped with a TXO Prodigy probe (500 MHz) in D₂O. ¹³C NMR spectra were recorded on a Bruker Avance III 500 MHz system equipped with DCH CryoProbe in D₂O.

Electrospray Ionization Mass Spectrometry (ESI-MS). ESI-MS spectra were collected on a Bruker Amazon-SL Mass-spectrometer configured with an ESI source in both negative and positive ionization mode.

Dynamic Light Scattering (DLS) measurements were performed on a DynaPro Nanostar (Wyatt Technology Corp, 633 nm laser) at room temperature in ultrapure water using disposable cuvettes.

Zeta potential was measured on a Malvern Zetasizer in ultrapure water at room temperature.

UV-Vis spectra were collected on a Cary Series 100 UV-vis spectrophotometer in a quartz cuvette.

Fourier Transform Infrared (FTIR) spectra were collected under transmission mode on a Nexus 870 spectrometer (Thermo Nicolet) in NU Atomic and Nanoscale Characterization Experimental Center (NUANCE) at Northwestern University.

Scanning Electron Microscope (SEM) images were acquired on a Hitachi SU8030 at an accelerating voltage of 10 kV and an emission current of 15 μA. For nanoparticle samples, silicon chips were mounted onto aluminum SEM stubs with carbon tape. 2 μL of the sample dispersion in water was drop-casted onto the silicon and left to dry overnight, followed by coating with 10 nm of osmium prior to imaging. For pheomelanin films on glass substrates, the dry films were mounted onto aluminum SEM stubs using carbon tape and then coated with 10 nm osmium prior to imaging.

Scanning Transmission Electron Microscopy (STEM) and Energy Dispersive Spectroscopy (STEM-EDS) images were obtained on a Hitachi HD2300 STEM operating at 200 kV. 400 mesh TEM grids were surface plasma treated using a PELCO easiGlow glow discharge cleaning system prior to use. 2 μL of the pheomelanin sample suspended in water was dropcasted onto a TEM grid and left to dry. EDS images were obtained using a frame time of 10.0 s and a dwell time of 200 s, and the acquisition stopped after 100 frames.

Methods

Synthesis of 5-cysteinyl-DOPA (5-CD)

5-CD was synthesized according to a previous literature preparative procedure.¹ Briefly, the Michael addition of cysteine to oxidized L-DOPA was performed in strong acidic conditions to inhibit further oxidation and polymerization. Afterward the crude product was purified twice by DOWEX 50 w×8 column to get rid of the _{L}-DOPA, 2-cysteinyl-DOPA and 2,5-dicysteinyl-DOPA. Then a reverse-phase HPLC was used to purify the compound. The pure compound was obtained as white powder. For detailed characterization of 5-CD, see FIGS. 9A-9D.

¹H NMR (500 MHz, Deuterium Oxide, ppm) δ 6.98 (d, J=2.1 Hz, 1H), 6.86 (d, J=2.1 Hz, 1H), 4.18 (dd, J=7.6, 5.5 Hz, 1H), 4.03 (t, J=5.7 Hz, 1H), 3.44 (d, J=5.7 Hz, 2H), 3.19 (dd, J=14.6, 5.5 Hz, 1H), 3.07 (dd, J=14.7, 7.7 Hz, 1H).

¹³C NMR (126 MHz, Deuterium Oxide, ppm) δ 171.75, 170.64, 144.90, 144.80, 126.83, 126.71, 118.48, 117.65, 54.32, 52.35, 34.88, 33.89.

¹⁹F NMR (470 MHz, Deuterium Oxide, ppm) δ −75.6.

ESI-MS: calculated for C₁₂H₁₇N₂O₆S [M+H]⁺ 317.08, found 317.06.

Polymerization of 5-CD to Obtain the Synthetic Pheomelanin

Pheomelanin was synthesized following a previously reported procedure.² In a typical experiment, 24 mg 5-CD was dissolved in 6 mL 100 mM DPBS in a 15 mL falcon tube. To this solution, 4.8 mg of HRP was added to afford a final concentration of 100 U/mL. Then, 45 mg ZnSO₄·7H₂O and 54 μL hydrogen peroxide (concentration 30% v/v) was added to the reaction system. The mixture was stirred at room temperature for 24 h. To work up the reaction, it was centrifuged at 11000 rpm for 10 min, treated with 1% acetic acid and then washed with ultrapure water. The pheomelanin pigment was obtained as reddish dispersion with yield ˜17% (the yield was based on the mass ratio of the final pheomelanin powder to the starting material 5-CD). We observed that the synthetic pheomelanin after lyophilization was difficult to redisperse in water, similar to PDA type eumelanin.

UV-Vis Spectroscopy Monitoring of the Reaction

To monitor the reaction using UV-vis spectrometry, a 20 μL aliquot was taken from the reaction at desired intervals and diluted in 400 μL deionized water before measurement to meet the detection limit of the UV-vis spectrometry. The absorption spectrum was measured immediately to minimize the reaction lag after dilution since no quenching agent is used for this experiment.

High-Performance Liquid Chromatography (HPLC) and MS Analysis

For HPLC monitoring, a 100 μL aliquot was diluted with 300 μL deionized water at the same stages of the reaction as the UV-vis measurements. 400 μL NaBH₄ aqueous solution (2 mg/mL, 0.05 mol/L) was added to quench the aliquoted reaction. Analytical HPLC analysis of peptides was performed on a Jupiter 4 Proteo 90 Å Phenomenex column (150×4.60 mm) using a Hitachi-Elite LaChrom L-2130 pump equipped with UV-vis detector (Hitachi-Elite LaChrom L2420). The solvent gradient for HPLC was 0-60% acetonitrile in 30 min. To analyze the different species, fractions were collected manually and analyzed on a Bruker Amazon-SL Mass-spectrometer.

Pheomelanin Extraction from the Covert Feathers of a Rhode Island Red Rooster or Human Red Hair

We isolated intact melanosomes from rooster feathers and human red hair following the enzymatic (Proteinase-K based) extraction method of Liu et al (2004).³ Compared to other harsher methods traditionally employed in melanin extraction (e.g. acid-base extraction), this method is expected to retain the integrity of the chemical composition of melanin. Taking the human red hair I as an example, briefly, 35 g of hair was washed sequentially with organic solvents, including acetone, dichloromethane, and ether, and then with ultrapure water. Then the hair was treated with Dithiothreitol (DTT, 1 g) and Proteinase-K (90 mg, 2700 U) in DPBS under nitrogen atmosphere for 48 h at 40° C. After centrifugation, the hair was treated with papain (50 mg) and DTT (1 g) and incubated with continuous nitrogen for 72 h at 60-70° C. The sample was collected by centrifuge and washed 6 times with deionized water. Afterward, the pellet was redispersed in DPBS containing proteinase-K (30 mg, 900 U) and DTT (100 mg) under nitrogen atmosphere for 48 h at 40° C. The sample was stirred at room temperature in 2% Triton™ X-100 for 4 h and then washed with 2× water, 2× methanol and 2× water. The sample was then treated with proteinase-K (80 mg) and DTT (400 mg) at 40° C. with continuous nitrogen flow. The pheomelanin from human red hair was obtained as a red to brown dispersion in water and lyophilized overnight for ssNMR. The total amount of pheomelanin obtained from the human red hair I was more than 200 mg.

The Preparation of PDA Eumelanin Control

PDA nanoparticles were synthesized by the auto-oxidation of dopamine hydrochloride by air under alkaline conditions as reported before.⁴ After reacting overnight, the samples were centrifuged at 11,000 rpm for 10 min followed by washing with ultrapure water for 3 times.

Cross-Polarization Magic-Angle Spinning ¹³C Solid-State NMR

20-80 mg of lyophilized melanin sample was packed into a H14355 4.0 mm magic-angle spinning (MAS) rotor from Bruker. The temperature was set to 298 K. Each 1D ¹³C cross polarization (CP) was acquired using 3 k-13 k scans (depending on the amount of sample) and a recycle delay of 5.0 s on a 400 MHz Bruker spectrometer. The CP contact time was optimized to 3 ms. All ¹³C NMR spectra were recorded with complete proton decoupling of 83 kHz. The spinning speed was 10 kHz. ¹³C chemical shifts were referenced to adamantane external reference at 38.3 ppm and reported in parts per million (ppm). FID files were processed using MestRenova 7 software (Mestrelab Research).

Note: the intrinsic heterogeneous structure of polymeric melanin results in broad linewidth in the NMR spectra.

UVA Irradiation of 5-CD Pheomelanin

The powder sample of 5-CD pheomelanin was irradiated with UV lamp (λ˜365 nm, 7.0 mW/cm²) for 30 h. The experimental condition was chosen to match the previous report by Wakamatsu (UVA dose 4.0 mW/cm², radiation time: 56 h).⁵ Then ssNMR were collected on a 400 MHz Bruker spectrometer.

XPS Experiment

X-ray photoelectron spectroscopy (XPS) spectra were collected on a Thermo Scientific ESCALAB 250Xi. For nanoparticles, samples were drop-casted onto a silicon substrate. Film samples on glass substrate were used as prepared. All XPS spectra were calibrated with reference to the carbon C1s peak at 284.8 eV.

EPR Experiment

Continuous wave EPR measurements (CW EPR) were performed at X-band (9.5 GHz) fields using a Bruker Elexsys E680 spectrometer equipped with a 4122SHQE resonator. Scans were performed with a magnetic field modulation amplitude of 2 G and non-saturating microwave power of 1.544 mW. The results are the average of 32 scans. Dispersion samples were contained in quartz tubes with I.D. 1.50 mm and O.D. 1.80 mm and measured at room temperature. For quantification, 4-amino-TEMPO was dissolved in ultrapure water as the spin standard. EPR spectra for solution samples were taken under identical conditions as the standard. To quantify the spin concentrations, the EPR spectra were double integrated and then the double-integration areas were plotted against the spin concentration.

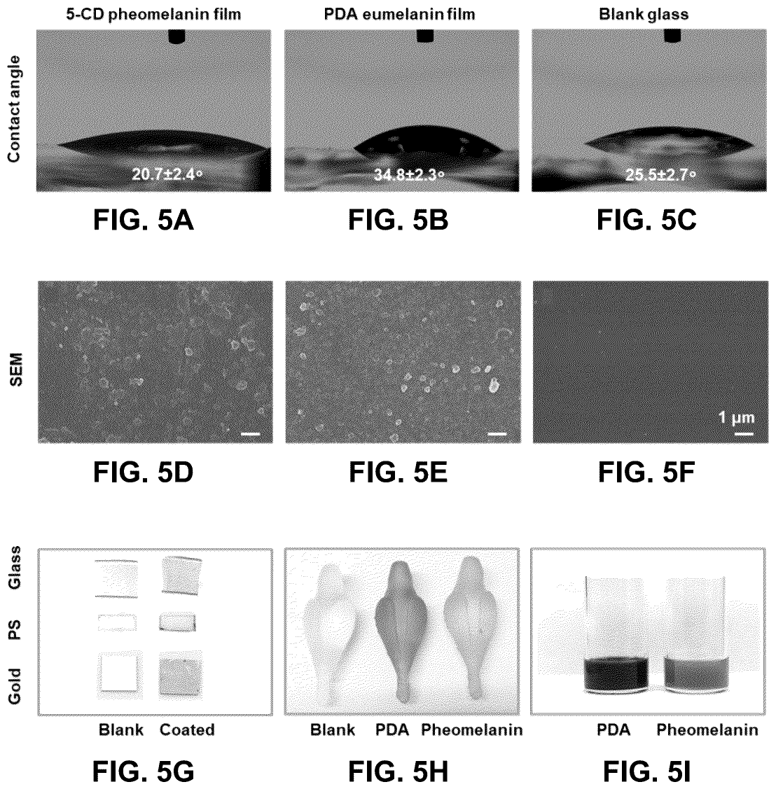

Solid samples and the pulse EPR measurements were also contained in quartz tubes with I.D. 1.50 mm and O.D. 1.80 mm and measured at room temperature on an E680 X/W EPR spectrometer with a split ring resonator (ER4118X-MS3). A 1 kW TWT amplifier (Applied Systems Engineering 117X) was used to generate high-power microwave pulses resulting in pulses, π/2=16 ns and π=32 ns. The resonator was partially over-coupled to maximize echo intensity and minimize ringing following microwave pulses. Spin-lattice relaxation times (T₁) of the PDA-type eumelanin and pheomelanin samples (synthetic and natural ones extracted from bird feathers) were determined using the saturation recovery technique (FIGS. 5A-5I). The spin ensemble of interest was saturated with a series of eight 24 ns pulses spaced 2 μs apart and the recovery was monitored at logarithmically spaced delays T starting with 100 ns and a pulse π/2-τ-π-τ-echo detection sequence, in which π/2=16 ns, π=32 ns and τ=200 ns. The signals recovered in exponential fashion and were fit using a well-established relaxation model.⁶

For the power saturation curve, X axis is the square root of incident power P. Y axis is the intensity values of the EPR maximum peak. P is calculated from the following equations:

$$\frac{P}{{P}_{0}} = {10}^{- \frac{d B}{10}}$$

In which P₀ is 196.2 mW.

The Preparation of Pheomelanin Film

For pheomelanin film preparation, a glass slide was cleaned with deionized water and immersed in the polymerization reaction of 5-CD for 24 h. Afterward, the glass was taken out, rinsed with ultrapure water for 3 times and then immersed in 1% acetic acid for 20 min followed by another 3 rounds of washing with ultrapure water. We note here that glass slides cleaned by piranha solution have much lower absorbance after pheomelanin film deposition than untreated glass slides.

The film from natural pheomelanin was prepared by drop casting of a drop of natural pheomelanin dispersion onto a clean glass slide.

The Preparation of Eumelanin Film Control

To prepare PDA-type eumelanin film, 2 mg/mL solution of dopamine was prepared. Around 180 μL of 0.2 M NaOH solution were added to adjust to pH 8.8. Glass slides were submerged in the solution for 24 h to allow the eumelanin film deposition. The oxidative polymerization was trigger by ambient oxygen under the alkali condition.

The coating of 3D-printed objects

The 3D-printed poly-methacrylate birds were printed with a Formlabs Form2 printer using Formlabs Clear V4 resin. The bird design was downloaded from https://free3d.com/3d-model/bird-v1--875504.html, and used under the non-commercial personal use license as an example part. After printing, the birds were rinsed in two successive isopropyl alcohol baths for 10 minutes each, then allowed to dry overnight before the pheomelanin coating using the as-described method.

To prepare PDA-type eumelanin coated birds, we use 2 mg/mL solution of dopamine was prepared in a pH 8.5 Tris buffer solution.

Reflectance Spectrophotometry

We used UV-vis spectrophotometry to characterize the reflectance of pheomelanin films. We measured specular reflectance between 300-700 nm using an Avantes (Avantes Inc., Boulder, CO, USA) AvaSpec-2048 spectrometer and an AvaLight-XE pulsed xenon light source, relative to a glass slide as reference. The spectral data were collected at a 90° angle of incidence for both the light and probe using AvaSoft v7.2.

Film Disassembly Studies

The as-prepared film on glass substrate was cut to around 3 mm or 5 mm squares by a diamond knife. The films were put in 96-well plate and submerged with 200 μL salt solution. Solutions used included water, pH 10 KOH solution, and pH 10 solution with KCl, NaCl, KBr, and K₂S04. The 96-well plated was placed under darkness for 24 h. Then each film was washed three times by ultrapure water. The absorbance mappings of the film samples were recorded using a Perkin Elmer EnSpire multimode Plate Reader. The absorbance at 400 nm was plot by MATLAB using a blank well to subtract the background.

### REFERENCES CORRESPONDING TO EXAMPLE 1B

1. Chioccara, F.; Novellino, E., A Convenient One Step Synthesis of 5-Cystein-S-yldopa Using Ceric Ammonium Nitrate. Synth. Commun. 1986, 16, (8), 967-971.
2. Napolitano, A.; De Lucia, M.; Panzella, L.; d'Ischia, M., The “benzothiazine” chromophore of pheomelanins: a reassessment. Photochem. Photobiol. 2008, 84, (3), 593-9.
3. Liu, Y.; Kempf, V. R.; Brian Nofsinger, J.; Weinert, E. E.; Rudnicki, M.; Wakamatsu, K.; Ito, S.; Simon, J. D., Comparison of the Structural and Physical Properties of Human Hair Eumelanin Following Enzymatic or Acid/Base Extraction. Pigment Cell Res. 2003, 16, (4), 355-365.
4. Huang, Y.; Li, Y.; Hu, Z.; Yue, X.; Proetto, M. T.; Jones, Y.; Gianneschi, N. C., Mimicking Melanosomes: Polydopamine Nanoparticles as Artificial Microparasols. ACS Cent. Sci. 2017, 3, (6), 564-569.
5. Wakamatsu, K.; Nakanishi, Y.; Miyazaki, N.; Kolbe, L.; Ito, S., UVA-induced oxidative degradation of melanins: fission of indole moiety in eumelanin and conversion to benzothiazole moiety in pheomelanin. Pigment Cell Melanoma Res 2012, 25, (4), 434-45.
6. Chen, H.; Maryasov, A. G.; Rogozhnikova, O. Y.; Trukhin, D. V.; Tormyshev, V. M.; Bowman, M. K., Electron spin dynamics and spin-lattice relaxation of trityl radicals in frozen solutions. Phys. Chem. Chem. Phys. 2016, 18, (36), 24954-24965.
7. Thureau, P.; Ziarelli, F.; Thevand, A.; Martin, R. W.; Farmer, P. J.; Viel, S.; Mollica, G., Probing the motional behavior of eumelanin and pheomelanin with solid-state NMR spectroscopy: new insights into the pigment properties. Chem. Eur. J. 2012, 18, (34), 10689-700.

### ADDITIONAL REFERENCES

Battistella, C.; McCallum, N. C.; Gnanasekaran, K.; Zhou, X.; Caponetti, V.; Montalti, M.; Gianneschi, N. C., Mimicking Natural Human Hair Pigmentation with Synthetic Melanin. ACS Cent. Sci. 2020, 6 (7), 1179-1188.
Battistella, C.; McCallum, N. C.; Vanthournout, B.; Forman, C. J.; Ni, Q. Z.; La Clair, J. J.; Burkart, M. D.; Shawkey, M. D.; Gianneschi, N. C., Bioinspired Chemoenzymatic Route to Artificial Melanin for Hair Pigmentation. Chem. Mater. 2020, 32 (21), 9201-9210.

Each of the references cited herein is hereby incorporate by reference in their entirety.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every system, composition, formulation, combination of components, step, and method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method for matching hair composition, the method comprising:
characterizing one or more first characteristics of a natural melanin composition of a hair sample from a subject; and
preparing a prepared artificial melanin formulation to approximate the one or more first characteristics;
wherein the prepared artificial melanin formulation comprises one or more artificial melanin materials.

2. The method of claim 1, further comprising determining a theoretical artificial melanin formulation to approximate the one or more characteristics of the natural melanin composition;
wherein the step of preparing comprises preparing the prepared artificial melanin formulation according to the theoretical artificial melanin composition.

3. The method of claim 1, wherein the step of characterizing comprises analyzing the hair sample using at least one technique selected from the group consisting of:
optical absorption spectroscopy, Fourier transform infrared (FTIR) spectroscopy, nuclear magnetic resonance (NMR) spectroscopy, mass spectroscopy (MS), electrospray ionization mass spectroscopy (SI-MS), dynamic light scattering (DLS), Zeta potential, electron microscope, scanning electron microscopy (SEM), energy dispersive spectroscopy (EDS), Raman spectroscopy, electron paramagnetic resonance (EPR) spectroscopy, ultraviolet-visible spectroscopy (UV-Vis), x-ray photoelectron spectroscopy (XPS), thermogravimetric analysis (TGA), differential scanning calorimetry (DSC), matrix assisted laser desorption/ionization (MALDI), and any combination of these.

4. The method of claim 1, wherein at least one of the one or more first characteristics of the natural melanin composition is selected from the group consisting of:
a concentration of one or more pheomelanins;
a concentration ratio of one or more pheomelanins relative to all melanin in the natural melanin composition;
a concentration and/or concentration ratio of one or more eumelanins;
a concentration ratio of one or more eumelanins relative to all melanin in the natural melanin composition;
a concentration and/or concentration ratio of one or more allomelanins;
a concentration ratio of one or more allomelanins relative to all melanin in the natural melanin composition;
chemical identity or formula of one or more pheomelanins, one or more eumelanins, and/or one or more allomelanins in the hair sample;
an optical absorption spectrum;
an FTIR spectrum;
an NMR spectrum;
a relative elemental composition with respect to two or more elements;
a mass spectrum;
a DLS spectrum;
a Zeta potential data set;
a Raman spectrum;
an EPR spectrum;
contact angle;
and any combination of these.

5. The method of claim 1, wherein the step of preparing comprises synthesizing at least a portion of the one or more artificial melanin materials.

6. The method of claim 5, wherein the step of preparing further comprises mixing the one or more artificial melanin materials to form an artificial melanin mixture; wherein the prepared artificial melanin formulation comprises the artificial melanin mixture.

7. The method of claim 1, wherein the prepared artificial melanin formulation is characterized by one or more third characteristics each being approximately equivalent to the respective first characteristic of the natural melanin composition.

8. The method of claim 7, wherein the one or more third characteristic (of the prepared melanin formulation) is within 10% error and/or has at least 70% spectral matching with the first characteristic (of the natural melanin composition).

9. The method of claim 1, wherein the prepared artificial melanin formulation has the same color as the natural melanin composition or wherein color of hair treated with the prepared artificial melanin formulation has the same color as the natural melanin composition or the hair sample.

10. The method of claim 2, wherein the theoretical artificial melanin formulation is characterized by one or more second characteristics each being approximately equivalent to the respective first characteristic of the natural melanin composition.

11. The method of claim 10, wherein the step of determining comprises determining one or more formulation design parameters of the theoretical artificial melanin formulation which result in the one or more second characteristics being approximately equivalent to the one or more first characteristics, respectively.

12. The method of claim 11, wherein the one or more formulation design parameters are selected from the group consisting of:
a desired concentration and/or desired concentration ratio of one or more artificial melanin materials characterized as pheomelanin;
a desired concentration and/or desired concentration ratio of one or more artificial melanin materials characterized as eumelanin;
a desired concentration and/or desired concentration ratio of one or more artificial melanin materials characterized as allomelanin;
degree of polymerization of the melanin;
one or more desired size characteristics of the one or more artificial melanin materials;
one or more desired structural characteristics of the one or more artificial melanin materials;
one or more desired optical characteristics of the one or more artificial melanin materials;
one or more desired radical quenching characteristics of the one or more artificial melanin materials;
and any combination of these.

13. The method of claim 10, wherein the one or more second characteristics (of the theoretical melanin formulation) is within 10% error and/or has at least 70% spectral matching with the first characteristic (of the natural melanin composition).

14. The method of claim 1, wherein the theoretical and prepared artificial melanin compositions comprise one or more artificial eumelanins, one or more artificial allomelanins, one or more artificial pheomelanins, and a combination of these.

15. The method of claim 1, wherein each of the one or more melanin materials is not bound to, conjugated to, attached to, coated by, encompassed by, or otherwise chemically associated with a natural or biological proteinaceous matrix, component, or lipid.

16. The method of claim 1, wherein at least a portion of the one or more artificial melanin materials is characterized as eumelanin, pheomelanin, allomelanin, or a combination of these.

17. The method of claim 1, wherein the one or more artificial melanin materials comprise a porous artificial melanin material.

18. The method of claim 1, wherein at least a portion of the one or more artificial melanin materials comprises a plurality of melanin polymers; and wherein each melanin polymer comprises a plurality of covalently-bonded melanin base units.

19. The method of claim 18, wherein said melanin base units are one or more substituted or unsubstituted catechol-based monomer units, substituted or unsubstituted polyol-based monomer units, substituted or unsubstituted phenol-based monomer units, substituted or unsubstituted indole-based monomer units, substituted or unsubstituted benzothiazine-based monomer units, substituted or unsubstituted benzothiazole-based monomer units, substituted or unsubstituted dopamine-based monomer units, or any combination of these.

20. The method of claim 18, wherein at least a portion of said melanin base units each independently comprises substituted or unsubstituted naphthalene.

21. The method of claim 18, wherein at least a portion of the one or more artificial melanin materials comprises at least one dihydoxyindole (DHI) (e.g., 5,6-dihydroxyindole), at least one dihydroxyindole-2-carboxylic acid (DHICA) (e.g., 5,6-dihydroxyindole-2-carboxylic acid), or a combination of these.

22. The method of claim 18, wherein at least 50% of the plurality of melanin polymers are selected from the group consisting of dimers, trimers, tetramers, pentamers, and any combination thereof.

23. The method of claim 18, wherein each melanin oligomer is non-covalently associated with at least one other melanin oligomer or a melanin monomer via at least one of hydrogen bonding and π-π stacking of naphthalene rings; wherein the melanin monomer comprises the melanin base unit.

24. The method of claim 18, wherein the artificial melanin material comprises a porous artificial melanin material; and wherein the melanin oligomers and/or polymers of the porous artificial melanin material are arranged to form an internal structure having a plurality of pores; wherein the porous artificial melanin material is characterized by a pore volume per mass of material greater than or equal to 0.1 cm³/g and wherein at least a portion of said pores have at least one size dimension greater than or equal to 0.5 nm.

25. The method of claim 1, wherein at least a portion of the one or more artificial melanin materials comprises at least one substituted or unsubstituted benzothiazine, at least one substituted or unsubstituted benzothiazole, at least one substituted or unsubstituted benzoselenazole, at least one substituted or unsubstituted benzoselenazine, at least one derivative of any of these, or any combination of these.

26. The method of claim 1, wherein at least a portion of the artificial melanin material comprises one or more selenomelanin polymers; wherein the one or more selenomelanin polymers comprise a plurality of covalently bonded selenomelanin base units; and wherein a chemical formula of each of the one or more selenomelanin base units comprises at least one selenium atom.

27. The method of claim 26, wherein each selenomelanin polymer is a pheomelanin.

28. The method of claim 1, wherein at least a portion of the artificial melanin material comprises cation-π interactions.

29. The method of claim 1, wherein the one or more artificial melanin materials comprise artificial melanin nanoparticles.

30. The method of claim 29, wherein the artificial melanin nanoparticles comprise porous artificial melanin nanoparticles.

31. The method of claim 29, wherein each of the one or more artificial melanin nanoparticles is not bound to, conjugated to, attached to, coated by, encompassed by, or otherwise chemically associated with a natural or biological proteinaceous matrix, component, or lipid.

32. The method of claim 1, wherein the subject is a human or animal.

33. The method of claim 1, wherein the hair samples is human hair.

34. The method of claim 1, wherein the step of characterizing comprises extracting the natural melanin composition from the hair sample.

35. The method of claim 34, wherein the step of extracting is performed via chemical extraction, enzymatic extraction, or a combination of these.

36. The method of claim 1, wherein the prepared artificial melanin formulation is provided to an end-user.

37. The method of claim 1, wherein the end-user is an individual customer or a hair treatment facility.

38. The method of claim 1 further comprising treating hair of the same or different subject with the prepared artificial melanin formulation.

39. A method for matching hair composition, the method comprising:
characterizing one or more first characteristics of a natural melanin composition of a hair sample from a subject;
determining a theoretical artificial melanin formulation to approximate the one or more characteristics of the natural melanin composition;
preparing a prepared artificial melanin formulation according to the theoretical artificial melanin composition;
wherein the prepared artificial melanin formulation comprises one or more artificial melanin materials.
